(19) United States
(12) Patent Application Publication
(10) Pub. No.: US 2024/0141437 A1
(43) Pub. Date: May 2, 2024

(54) METHODS AND COMPOSITIONS FOR NEOADJUVANT AND ADJUVANT UROTHELIAL CARCINOMA THERAPY

(71) Applicant: Genentech, Inc., South San Francisco, CA (US)

(72) Inventors: Sanjeev MARIATHASAN, Millbrae, CA (US); Chi Yung YUEN, Millbrae, CA (US); Zoe June Fergusson ASSAF, San Francisco, CA (US); Carlos Ernesto BAIS, San Francisco, CA (US); Romain Francois BANCHEREAU, San Mateo, CA (US)

(21) Appl. No.: 18/327,305

(22) Filed: Jun. 1, 2023

Related U.S. Application Data
(63) Continuation of application No. PCT/US21/61185, filed on Nov. 30, 2021.
(60) Provisional application No. 63/210,950, filed on Jun. 15, 2021.
(60) Provisional application No. 63/120,643, filed on Dec. 2, 2020.

(51) Int. Cl. C12Q 1/6886; A61K 45/06; A61P 35/00; C07K 16/28; C12Q 1/686; C12Q 1/6869; A61K 39/00

(52) U.S. Cl. CPC C12Q 1/6886; A61K 45/06; A61P 35/00; C07K 16/2827; C12Q 1/686; C12Q 1/6869; A61K 2039/505; C12Q 2600/106; C12Q 2600/112; C12Q 2600/158

(57) ABSTRACT

The invention provides methods and compositions for treating urothelial carcinoma in a patient, for example, by administering a treatment regimen that includes a PD-1 axis binding antagonist (e.g., atezolizumab) to the patient as a neoadjuvant or an adjuvant therapy based on the presence or level of ctDNA in a biological sample obtained from the patient. Also provided are compositions (e.g., a PD-1 axis binding antagonist (e.g., atezolizumab), pharmaceutical compositions thereof, kits thereof, and articles of manufacture thereof) for use in treating urothelial carcinoma in a patient.

### CROSS-REFERENCE TO RELATED APPLICATIONS

This application is a continuation of International Patent Application No. PCT/US2021/061185, filed on Nov. 30, 2021, which claims benefit to U.S. Provisional Patent Application Ser. Nos. 63/120,643, filed on Dec. 2, 2020, and 63/210,950, filed on Jun. 15, 2021, the entire contents of which are incorporated herein by reference in their entirety.

### SEQUENCE LISTING

The instant application contains a Sequence Listing which has been submitted electronically in XML format and is hereby incorporated by reference in its entirety. Said XML copy, created on May 26, 2023, is named 50474-242004_Sequence_Listing_5_26_23 and is 10,426 bytes in size.

### FIELD OF THE INVENTION

This invention relates to methods and compositions for use in treating urothelial carcinoma (UC) in a patient, for example, by administering to the patient a treatment regimen that includes a PD-1 axis binding antagonist (e.g., atezolizumab).

### BACKGROUND OF THE INVENTION

UC is the most common cancer of the urinary system worldwide. The majority of cases originate in the bladder. UC can be diagnosed as non-muscle invasive, muscle-invasive, or metastatic disease, with 1 in 3 new cases diagnosed as muscle-invasive disease (cT2-T4a Nx M0 according to tumor, node, and metastasis (TNM) classification). Muscle-invasive UC (MIUC) collectively refers to muscle-invasive bladder cancer (MIBC) and muscle-invasive urinary tract urothelial cancer (UTUC). In 2018, there were an estimated 549,393 new cases of bladder cancer and 199,922 deaths worldwide. In Europe, it was estimated that there were 197,110 new cases of bladder cancer and 64,970 deaths, including 164,450 new cases and 52,930 deaths in the 28 member states of the European Union. In the United States, in 2020, it is estimated that there will be 81,400 new cases of bladder cancer and 17,980 deaths. Patients diagnosed with UC in the United States have a median age of 73, the highest age at diagnosis of all tumor types.

For MIBC, radical cystectomy with bilateral pelvic lymphadenectomy is the backbone of management. The surgery involves resection of the bladder, adjacent organs, and regional lymph nodes. There are also sex-based differences in the surgical approach: for men, the surgery includes removal of the prostate and seminal vesicles; and for women, the surgery includes removal of the uterus, cervix, ovaries, and anterior vagina. Urinary diversion is required after removal of the bladder. The perioperative mortality rate is approximately 2%-3% when cystectomy is performed at centers of excellence.

In spite of this surgery, MIBC recurs in many patients, and they present with pain or constitutional symptoms such as fatigue, weight loss, anorexia, and failure to thrive. Approximately half of the patients with MIBC will develop a local and/or metastatic recurrence of their disease within 2 years of cystectomy and will eventually die from their disease. For those with high-risk features (pT3-T4a or pN+) who have not received neoadjuvant chemotherapy (NAC), the overall 5-year survival ranges from 10% to 40%. Despite numerous attempted clinical trials, no adjuvant therapies to date have shown improved survival in MIBC.

Thus, there remains a need in the art for improved neoadjuvant and adjuvant treatment approaches for UC.

### SUMMARY OF THE INVENTION

The invention relates to, inter alia, methods, compositions (e.g., pharmaceutical compositions), uses, kits, and articles of manufacture for adjuvant treatment of UC.

In one aspect, the invention features a method of treating muscle-invasive urothelial carcinoma (MIUC) in a patient in need thereof, the method comprising administering to the patient an effective amount of a treatment regimen comprising an anti-PD-L1 antibody, wherein the anti-PD-L1 antibody comprises (a) a hypervariable region (HVR)-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively, wherein the treatment regimen is an adjuvant therapy, and wherein the patient has been identified as likely to benefit from the treatment regimen based on the presence of circulating tumor DNA (ctDNA) in a biological sample obtained from the patient.

In another aspect, the invention features a method of treating MIUC in a patient in need thereof, the method comprising: (a) determining whether ctDNA is present in a biological sample obtained from the patient, wherein the presence of ctDNA in the biological sample indicates that the patient is likely to benefit from a treatment regimen comprising an PD-L1 antibody; and (b) administering an effective amount of a treatment regimen comprising an PD-L1 antibody to the patient based on the presence of ctDNA in the biological sample, wherein the treatment regimen is an adjuvant therapy, and wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In another aspect, the invention features a method of identifying a patient having an MIUC who may benefit from a treatment regimen comprising an anti-PD-L1 antibody, the method comprising determining whether ctDNA is present in a biological sample obtained from the patient, wherein the presence of ctDNA in the biological sample identifies the patient as one who may benefit from treatment with a treatment regimen comprising an anti-PD-L1 antibody, wherein the treatment regimen is an adjuvant therapy, and wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In another aspect, the invention features a method for selecting a therapy for a patient having an MIUC, the method comprising (a) determining whether ctDNA is present in a biological sample obtained from the patient, wherein the presence of ctDNA in the biological sample indicates that the patient is likely to benefit from a treatment regimen comprising an anti-PD-L1 antibody; and (b) selecting a treatment regimen comprising an anti-PD-L1 antibody based on the presence of ctDNA in the biological sample, wherein the treatment regimen is an adjuvant therapy, and wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In another aspect, the invention features a method of monitoring the response of a patient having an MIUC who has been administered at least a first dose of a treatment regimen comprising an anti-PD-L1 antibody, wherein the treatment regimen is a neoadjuvant therapy or an adjuvant therapy, and wherein ctDNA was present in a biological sample obtained from the patient prior to or concurrently with the first dose of the treatment regimen, the method comprising determining whether ctDNA is present in a biological sample obtained from the patient at a time point following administration of the first dose of the treatment regimen, thereby monitoring the response of the patient, wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively. In some embodiments, the treatment regimen is a neoadjuvant therapy. In other embodiments, the treatment regimen is an adjuvant therapy.

In another aspect, the invention features a method of identifying a patient having an MIUC who may benefit from a treatment regimen comprising an anti-PD-L1 antibody, wherein the treatment regimen is a neoadjuvant therapy or an adjuvant therapy and the patient has been administered at least a first dose of the treatment regimen, and wherein ctDNA was present in a biological sample obtained from the patient prior to or concurrently with the first dose of the treatment regimen, the method comprising: determining whether ctDNA is present in a biological sample obtained from the patient at a time point following administration of the first dose of the treatment regimen, wherein an absence of ctDNA in the biological sample at the time point following administration of the treatment regimen identifies the patient as one who may benefit from treatment with a treatment regimen comprising an anti-PD-L1 antibody, wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively. In some embodiments, the treatment regimen is a neoadjuvant therapy. In other embodiments, the treatment regimen is an adjuvant therapy.

In another aspect, the invention features an anti-PD-L1 antibody, or a pharmaceutical composition comprising an anti-PD-L1 antibody, for use in treatment of an MIUC in a patient in need thereof, wherein the treatment comprises administration of an effective amount of a treatment regimen comprising an anti-PD-L1 antibody, wherein the treatment regimen is an adjuvant therapy, and wherein the patient has been identified as likely to benefit from the treatment regimen based on the presence of ctDNA in a biological sample obtained from the patient, and wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In another aspect, the invention features an anti-PD-L1 antibody, or a pharmaceutical composition comprising an anti-PD-L1 antibody, for use in treatment of MIUC in a patient in need thereof, the treatment comprising: (a) determining whether ctDNA is present in a biological sample obtained from the patient, wherein the presence of ctDNA in the biological sample indicates that the patient is likely to benefit from a treatment regimen comprising an anti-PD-L1 antibody; and (b) administering an effective amount of a treatment regimen comprising an anti-PD-L1 antibody to the patient based on the presence of ctDNA in the biological sample, wherein the treatment regimen is an adjuvant therapy, and wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In another aspect, the invention features an anti-PD-L1 antibody, or a pharmaceutical composition comprising an anti-PD-L1 antibody, for use in treatment of a patient having an MIUC who has been administered at least a first dose of a treatment regimen comprising an anti-PD-L1 antibody, wherein the treatment regimen is a neoadjuvant therapy or an adjuvant therapy, and wherein ctDNA was present in a biological sample obtained from the patient prior to or concurrently with the first dose of the treatment regimen, wherein the patient's response has been monitored by a method comprising determining whether ctDNA is present in a biological sample obtained from the patient at a time point following administration of the first dose of the treatment regimen, wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively. In some embodiments, the treatment regimen is a neoadjuvant therapy. In other embodiments, the treatment regimen is an adjuvant therapy.

### BRIEF DESCRIPTION OF THE DRAWINGS

FIG. 1A is a schematic diagram showing the inclusion criteria for the ctDNA biomarker-evaluable population (BEP) in the IMvigor010 study.

FIGS. 1B and 1C are a series of graphs showing Kaplan-Meier plots comparing patients treated with atezolizumab (dark gray) to observation (light gray) for the probability of disease-free survival (DFS) in the ctDNA BEP population, stratified for nodal status, PD-L1 status, and tumor stage (FIG. 1B), and interim probability of overall survival (OS) in the ctDNA BEP population, stratified for nodal status, PD-L1 status, and tumor stage (FIG. 1C). HR, hazard ratio.

FIGS. 2A-2D are a series of graphs showing Kaplan-Meier plots comparing ctDNA(+) (dark gray) to ctDNA(−) (light gray) status at C1D1 for DFS in the atezolizumab arm (FIG. 2A), DFS in the observation arm (FIG. 2B), OS in the atezolizumab arm (FIG. 2C), and OS in the observation arm (FIG. 2D). The probability of DFS and the probability of OS are shown on the y-axes. C1D1, Cycle 1 Day 1.

FIG. 3 is a histogram plot showing the distribution of durations between a C1D1 ctDNA(+) test and radiological relapse for patients within the C1D1 ctDNA(+) subgroup.

FIGS. 4A and 4B are a series of graphs showing Kaplan-Meier plots of DFS comparing ctDNA(+) patients treated with atezolizumab and ctDNA(+) patients on the observation arm, and comparing ctDNA(−) patients treated with atezolizumab and ctDNA(−) patients on the observation arm (FIG. 4A), and interim OS in patients evaluated for ctDNA status, comparing ctDNA(+) patients treated with atezolizumab and ctDNA(+) patients on the observation arm, and comparing ctDNA(−) patients treated with atezolizumab and ctDNA(−) patients on the observation arm (FIG. 4B). The probability of DFS and the probability of OS are shown on the y-axes.

FIGS. 5A and 5B are a series of forest plots showing DFS (FIG. 5A) and OS (FIG. 5B) in the BEP comparing atezolizumab versus observation in subgroups defined by established prognostic factors. Subgroups defined by baseline clinical features and tissue immune biomarkers including nodal status, tumor stage, the number of lymph nodes resected, previous neoadjuvant chemotherapy, PD-L1 status by tissue immunohistochemistry (IHC), TMB status by tissue whole-exome sequencing (WES), as well as transcriptomic signatures including tGE3, TBRS, angiogenesis, and TCGA subtypes are shown. Forest plots show HRs for recurrence or death estimated using a univariable Cox proportional-hazards model, and 95% confidence intervals of HRs are represented by horizontal bars.

FIG. 5C is bar plot showing association of baseline prognostic factors with ctDNA(−) status (light gray) and ctDNA(+) status (dark gray), wherein nodal-positive patients were enriched for ctDNA-positive status (nodal-positive patients were 47.5% ctDNA positive, and nodal-negative patients were 25.2% ctDNA positive).

FIGS. 6A and 6B are a series of forest plots showing DFS in atezolizumab versus observation for ctDNA(+) patients (FIG. 6A) and ctDNA(−) patients (FIG. 6B). Subgroups defined by baseline clinical features and tissue immune biomarkers including nodal status, tumor stage, number of lymph nodes resected, prior neoadjuvant chemotherapy, PD-L1 status by tissue IHC, TMB status by tissue WES, as well as transcriptomic signatures including tGE3, TBRS, Angiogenesis, and TCGA subtypes are shown. Forest plots show HRs for death estimated using a univariable Cox proportional-hazards model, and 95% confidence intervals of HRs are represented by horizontal bars.

FIGS. 7A and 7B are a series of forest plots showing OS in atezolizumab versus observation for ctDNA(+) patients (FIG. 7A) and ctDNA(−) patients (FIG. 7B). Subgroups defined by baseline clinical features and tissue immune biomarkers including nodal status, tumor stage, number of lymph nodes resected, prior neoadjuvant chemotherapy, PD-L1 status by tissue IHC, TMB status by tissue WES, as well as transcriptomic signatures including tGE3, TBRS, Angiogenesis, and TCGA subtypes are shown. Forest plots show HRs for death estimated using a univariable Cox proportional-hazards model, and 95% confidence intervals of HRs are represented by horizontal bars.

FIGS. 8A-8H are a series of graphs showing Kaplan-Meier plots for TMB or PD-L1 subgroups. FIGS. 8A and 8C are a series of graphs showing Kaplan-Meier plots for patients who are TMB(+) and on the atezolizumab arm, TMB(+) and on the observation arm, TMB(−) and on the atezolizumab arm, and TMB(−) and on the observation arm, for DFS in all ctDNA BEP patients (FIG. 8A), and OS in all ctDNA BEP patients (FIG. 8C). FIGS. 8B and 8D are a series of graphs showing Kaplan-Meier plots for patients who are TMB(+)/high and on the atezolizumab arm, TMB(+)/high and on the observation arm, TMB(−)/low and on the atezolizumab arm, and TMB(−)/low and on the observation arm, for DFS in ctDNA(+) patients (FIG. 8B) and for OS in ctDNA(+) patients (FIG. 8D). TMB was measured by WES. FIGS. 8E and 8G are a series of graphs showing Kaplan-Meier plots for patients who are PD-L1(+) and on the atezolizumab arm, PD-L1(+) and on the observation arm, PD-L1(−) and on atezolizumab arm, and PD-L1(−) and the observation arm, for DFS in all ctDNA BEP patients (FIG. 8E), and OS in all ctDNA BEP patients (FIG. 8G). FIGS. 8F and 8H are a series of graphs showing Kaplan-Meier plots for patients who are PD-L1(+)/high and on the atezolizumab arm, PD-L1(+)/high and on the observation arm, PD-L1(−)/low and on the atezolizumab arm, and PD-L1(−)/low and on the observation arm, for DFS in ctDNA(+) patients (FIG. 8F) and for OS in ctDNA(+) patients (FIG. 8H). TMB, tumor mutational burden. PD-L1 IC, PD-L1 expression on tumor-infiltrating immune cells (IC) by IHC.

FIGS. 9A and 9B are a series of graphs showing Kaplan-Meier plots for DFS in patients who are ctDNA(−) and TMB(+) in the atezolizumab arm and observation arm, and DFS in patients who are ctDNA(−) and TMB(−) in the atezolizumab arm and observation arm (FIG. 9A); and OS in patients who are ctDNA(−) and TMB(+) in the atezolizumab arm and observation arm, and OS in patients who are ctDNA(−) and TMB(−) in the atezolizumab arm and observation arm (FIG. 9B).

FIGS. 10A and 10B are a series of graphs showing Kaplan-Meier plots for DFS in patients who are ctDNA(−) and PD-L1(+) in the atezolizumab arm and observation arm, and DFS in patients who are ctDNA(−) and PD-L1(−) in the atezolizumab and observation (FIG. 10A); and OS in patients who are ctDNA(−) and PD-L1(+) in the atezolizumab arm and observation arm, and OS in patients who are ctDNA(−) and PD-L1(−) in the atezolizumab arm and observation arm (FIG. 10B).

FIGS. 11A-11D are a series of graphs showing Kaplan-Meier plots comparing ctDNA(+) (dark gray) to ctDNA(−) (light gray) status at C3D1 for DFS in the atezolizumab arm (FIG. 11A), OS in the atezolizumab arm (FIG. 11B), DFS in the observation arm (FIG. 11C), and OS in the observation arm (FIG. 11D).

FIG. 12A is a graph showing the proportion of patients who were ctDNA(+) at C1D1 who converted to ctDNA(−) by C3D1 (Pos>Neg; clearance) compared to those who remained ctDNA(+) at C3D1 (Pos>Pos) for the atezolizumab arm and the observation arm. C3D1, Cycle 3 Day 1; Pos, ctDNA(+); Neg, ctDNA(−).

FIGS. 12B-12E are a series of graphs showing Kaplan-Meier plots showing different ctDNA dynamics from C1D1 to C3D1 including patients who were ctDNA(+) at C1D1 and cleared ctDNA by C3D1 (Pos>Neg; dark solid lines), patients who were ctDNA(+) at C1D1 and did not clear ctDNA (Pos>Pos; dark dashed lines), patients who were ctDNA(−) at C1D1 and remained ctDNA(−) at C3D1 (Neg>Neg; light solid lines), and patients who were ctDNA(−) at C1D1 and became ctDNA(+) at C3D1 (Neg>Pos; light dashed line), for DFS in the atezolizumab arm (blue colors) (FIG. 12B), DFS in the observation arm (FIG. 12C), OS in the atezolizumab arm (FIG. 12D), and OS in the observation arm (FIG. 12E).

FIG. 12F is a bar plot showing the proportion of ABACUS study participants who were ctDNA(+) (dark gray) or ctDNA(−) (light gray), comparing patients who had response to atezolizumab neoadjuvant therapy (pathological complete response (pCR)/major pathological response (MPR), left) and patients who did not (non-responders, right). Pre-treatment and post-treatment time points are shown (x-axis).

FIG. 12G is a box plot showing ctDNA concentrations (sample MTM/mL) in ABACUS study participants who were ctDNA(+) and ctDNA(−), comparing patients who had response (pCR/MPR, left) to atezolizumab neoadjuvant therapy and patients who did not (non-responders, right). Pre-treatment and post-treatment time points are shown (x-axis). Sample sizes for the boxplots from left to right are n=17, 15, 23, and 15. The boxplots depict the median at the middle line, with the lower and upper hinges at the first and third quartiles, respectively, the whiskers showing the minima to maxima no greater than 1.5× the interquartile range, and the remaining outlying data points plotted individually.

FIG. 12H is a bar plot showing the fraction of ctDNA(+) patients who had ctDNA clearance (dark gray) or non-clearance (light gray) by the post-treatment time point, comparing patients who had response to atezolizumab neoadjuvant therapy (pCR/MPR, left) and patients who did not (non-responders, right).

FIG. 13A is a scatter plot showing the ctDNA concentration as measured by sample mean tumor molecules per mL of plasma (Sample MTM/mL) versus DFS in months. Solid points indicate an event, and empty points indicate censoring. Observation arm ctDNA(+) patients are shown.

FIG. 13B is a Kaplan-Meier plot showing DFS in patients with high ctDNA concentrations (dark gray; greater than or equal to median Sample MTM/mL (i.e., sample MTM/mL≥median)) versus low ctDNA concentrations (light gray; less than the median Sample MTM/mL (i.e., sample MTM/mL<median)). Observation arm ctDNA(+) patients are shown.

FIG. 13C is a forest plot showing DFS in patients with high versus low ctDNA levels using different quantile thresholds for splitting Sample MTM/mL, including a 10% quantile, 25% quantile, 50% (median) quantile, 75% quantile, and 90% quantile. Observation arm ctDNA(+) patients are shown. Forest plot shows HRs for recurrence or death estimated using a univariable Cox proportional-hazards model, and 95% confidence intervals of HRs are represented by horizontal bars.

FIG. 13D is a scatter plot showing OS in months (x-axis) versus ctDNA concentration as measured by Sample MTM/mL. Solid points indicate an event, and empty points indicate censoring. Observation arm ctDNA(+) patients are shown.

FIG. 13E is a Kaplan-Meier plot showing OS in patients with high ctDNA concentrations (dark gray; greater than or equal to median Sample MTM/mL (i.e., sample MTM/mL a median)) versus low ctDNA concentrations (light gray; less than the median Sample MTM/mL (i.e., sample MTM/mL<median)). Observation arm ctDNA(+) patients are shown.

FIG. 13F is a forest plot showing OS in patients with high versus low ctDNA concentrations using different quantile thresholds for splitting ctDNA Sample MTM/mL, including a 10% quantile, 25% quantile, 50% (median) quantile, 75% quantile, and 90% quantile. Observation arm ctDNA(+) patients are shown. Forest plot shows HRs for recurrence or death estimated using a univariable Cox proportional-hazards model, and 95% confidence intervals of HRs are represented by horizontal bars.

FIG. 14A is a bar plot showing the percent of patients who were ctDNA(+) at C1D1 that had reduced ctDNA by C3D1 in the atezolizumab arm (dark gray) and the observation arm (light gray). Reduction was assessed in C1D1 ctDNA(+) patients in the C1/C3 BEP and defined as a decrease in sample MTM/mL from C1 to C3.

FIGS. 14B-14E are a series of Kaplan-Meier plots showing patients who had reduction in ctDNA (“reduction” (decrease); dark gray) compared with those who had ctDNA levels that increased (“non-reduction” (increase); light hrasy) for DFS in the atezolizumab arm (FIG. 14B), DFS in the observation arm (FIG. 14C), OS in the atezolizumab arm (FIG. 14D), and OS in the observation arm (FIG. 14E). Reduction was assessed in C1D1 ctDNA(+) patients in the C1/C3 BEP and defined as a decrease in sample MTM/mL from C1D1 to C3D1.

FIG. 15A is a Kaplan-Meier plot showing DFS wherein ctDNA reduction is split into patients who cleared ctDNA (“reduction with clearance”; dark gray, solid line) and those who had decreased ctDNA without clearance (“reduction without clearance”; dark gray, dashed line). Patients with an increase in ctDNA are also shown (“increase”; light gray, solid line).

FIG. 15B is a forest plot showing DFS comparing patients with ctDNA reduction (from clearance (−100% change) to minor decreases in ctDNA (<0% change)) using different thresholds for percent change in Sample MTM/mL, including −100% change (reduction with clearance versus reduction without clearance), −50% change, −25% change, and −10% change. Note that the scale for percent change goes from −100% (clearance) to infinity, where negative values indicate reductions, and positive values indicate increases.

FIG. 15C is a Kaplan-Meier plot showing OS wherein ctDNA reduction is split into patients who cleared ctDNA (“reduction with clearance”; dark gray, solid line) and those who had decreased ctDNA without clearance (“reduction without clearance”; dark gray, dashed line). Patients with an increase in ctDNA are also shown (“increase”; light gray, solid line).

FIG. 15D is a forest plot showing OS comparing patients with ctDNA reduction (from clearance (−100% change) to minor decreases in ctDNA (<0% change)) using different thresholds for percent change in Sample MTM/mL, including −100% change (reduction with clearance versus reduction without clearance), −50% change, −25% change, and −10% change. Note that the scale for percent change goes from −100% (clearance) to infinity, where negative values indicate reductions, and positive values indicate increases.

FIG. 16A is a scatter plot showing ctDNA concentrations (C1D1 sample MTM/mL) versus C1D1 collection time (days after surgery) in muscle-invasive bladder cancer (MIBC) patients.

FIG. 16B is a box plot showing the C1D1 collection time (y-axis, days after surgery) for the ctDNA-negative (x-axis, left box plot, n=339) and ctDNA-positive (x-axis, right box plot, n=199) MIBC patients. No difference was found between the collection times for the ctDNA-negative patients and the ctDNA-positive patients (Wilcoxon P=0.18, two sided). The boxplot middle line is the median, the lower and upper hinges correspond to the first and third quartiles, the upper whisker extends from the hinge to the largest value no further than 1.5×IQR from the hinge and the lower whisker extends from the hinge to the smallest value at most 1.5×IQR of the hinge, while data beyond the end of the whiskers are outlying points that are plotted individually.

FIG. 16C is a bar plot showing the fraction of patients who were ctDNA positive (dark gray fill) for patients with C1D1 collection times less than the median collection time (x-axis, left bar plot) and greater than the median collection time (x-axis, right bar plot). MIBC patients are shown.

FIG. 16D is a histogram showing the time between surgery and C1 D1 (days) for MIBC patients.

FIG. 17A is a consort diagram showing how patients in the ctDNA biomarker-evaluable population (BEP, n=40) were identified from the overall ABACUS study population (n=95).

FIG. 17B is a Kaplan-Meier plot comparing recurrence free survival of ctDNA-positive patients (light gray) to ctDNA-negative patients (dark gray) as assessed at the baseline (C1D1) time point prior to neoadjuvant treatment.

FIG. 17C is a Kaplan-Meier plot comparing recurrence free survival of ctDNA-positive patients (light gray) to ctDNA-negative patients (dark gray) as assessed at the post-neoadjuvant time point.

FIG. 18A is a volcano plot showing differential gene expression analysis in the ctDNA BEP indicating genes associated with ctDNA positivity (ctDNA+) and ctDNA negativity (ctDNA−).

FIG. 18B is a graph showing hallmark gene set enrichment analysis results in the ctDNA BEP indicating pathways associated with ctDNA positivity (ctDNA+; dark gray) and ctDNA negativity (ctDNA−; light gray).

FIG. 18C is a graph showing hallmark gene set enrichment analysis results in the ctDNA(+) patients in the atezolizumab arm showing pathways associated with relapse and non-relapse. DN, down; EMT, epithelial mesenchymal transition.

FIGS. 18D-18F are a series of Kaplan-Meier plots showing OS for ctDNA(+) patients in the atezolizumab and observation arms in subgroups defined by immune biomarkers of response (FIG. 18D) and resistance (FIGS. 18E and 18F) to immunotherapy. Immunotherapy response biomarker tGE3 gene expression signature (FIG. 18D) is shown. Immune biomarkers of resistance to immunotherapy pan-TBRS gene expression signature (FIG. 18E), and Angiogenesis gene expression signature (FIG. 18F) are shown. High biomarker expression is indicated in darker shading. Low biomarker expression is indicated in lighter shading.

FIGS. 19A-19C are a series of Kaplan-Meier plots showing DFS for ctDNA(+) patients in the atezolizumab and observation arms in subgroups defined by immune biomarkers of response (FIG. 19A) and resistance (FIGS. 19B and 19C) to immunotherapy. Immunotherapy response biomarker tGE3 gene expression signature (FIG. 19A) is shown. Immune biomarkers of resistance to immunotherapy pan-TBRS gene expression signature (FIG. 19B), and Angiogenesis gene expression signature (FIG. 19C) are shown. High biomarker expression is indicated in darker shading. Low biomarker expression is indicated in lighter shading.

FIG. 19D is a graph showing hallmark gene set enrichment analysis results in ctDNA+ patients in the observation arm comparing non-relapsers (light gray) to relapsers (dark gray).

FIGS. 20A-20C are a series of Kaplan-Meier plots showing ctDNA(−) patients in the atezolizumab and observation arms for DFS (left) and OS (right). Transcriptomic signatures including tGE3 (FIG. 20A), pan F-TBRS (FIG. 20B), and Angiogenesis (FIG. 20C) are shown. High biomarker expression is indicated in darker shading. Low biomarker expression is indicated in lighter shading.

FIG. 21A is a heatmap showing that hierarchical clustering in the ctDNA biomarker evaluable population recapitulates TCGA subtypes for urothelial carcinoma. APM, antigen-presenting machinery; ECM, extracellular matrix; IC, tumor-infiltrating immune cells; TC, tumor cells.

FIGS. 21B-21E are a series of Kaplan-Meier plots showing OS for patients in the atezolizumab and observation arms. Prognostic and/or predictive value of ctDNA status and TCGA subtype in the ctDNA BEP for Luminal papillary (FIG. 21B), Luminal infiltrated (FIG. 21C), Luminal (FIG. 21D), and Basal/Squamous (FIG. 21E) are shown. ctDNA(−) status and ctDNA(+) status are indicated.

FIG. 21F is a volcano plot showing differential gene expression analysis in observation (Obs) arm ctDNA(−) patients showing genes associated with relapse (left) and non-relapse (right). ECM, extracellular matrix. IFN, interferon.

FIG. 21G is a graph showing hallmark gene set enrichment analysis results in observation arm (Obs) ctDNA(−) patients showing pathways associated with relapse and non-relapse.

FIGS. 21H and 21I are a series of bar plots in ctDNA(−) patients (arms combined) showing distribution of TCGA subtypes binned by relapse (left) or non-relapse (right) (FIG. 21H), and relapsing patients (arms combined) showing fraction of patients that are ctDNA(+) (dark gray) and ctDNA(−) (light gray) binned by either distant relapse (left) or local relapse (right) (FIG. 21I).

FIGS. 22A and 22B are a series of bar plots showing the distribution of patients in TCGA subgroups compared between ctDNA(−) and ctDNA(+) populations (FIG. 22A) and compared between PD-L1 status populations (IC01 and IC23) (FIG. 22B).

FIGS. 22C-22H are a series of Kaplan-Meier plots showing DFS for ctDNA(+) (dark shading) and ctDNA(−) (light shading) patients in atezolizumab and observation arms for TCGA subgroups (FIGS. 22C-22F), and DFS (FIG. 22G) and OS (FIG. 22H) in the neuronal TCGA subgroup.

FIG. 23 shows a study schema for the IMvigor011 phase III, double-blind, randomized study of atezolizumab versus placebo as adjuvant therapy in patients with high-risk muscle-invasive bladder cancer who are ctDNA-positive following cystectomy. Min., minimum; NAC, neoadjuvant chemotherapy; SOC, standard of care; Cx, cystectomy; WES, whole-exome sequencing.

### DETAILED DESCRIPTION OF THE INVENTION

The present disclosure provides therapeutic methods and compositions for urothelial carcinoma. The present invention is based, at least in part, on the discovery that ctDNA positivity at baseline was associated with significantly improved DFS and OS in urothelial carcinoma patients receiving adjuvant therapy comprising a PD-1 axis binding antagonist (e.g., an anti-PD-L1 antibody such as atezolizumab) in a prospective analysis in the phase III IMvigor010 study (see, e.g., Example 1). The present invention is also based, at least in part, on the discovery that rates of ctDNA clearance were higher in patients receiving neoadjuvant therapy or adjuvant therapy comprising a PD-1 axis binding antagonist compared to observation, and clearance was associated with improved DFS and OS in the phase III IMvigor010 study and in the phase II ABACUS study of neoadjuvant atezolizumab therapy (see, e.g., Example 1). Thus, the methods and compositions provided herein allow for identification and treatment of patients who may benefit from neoadjuvant or adjuvant therapy comprising a PD-1 axis binding antagonist (e.g., atezolizumab), including patients with MIBC (e.g., high-risk MIBC) who are ctDNA-positive following surgical resection (e.g., cystectomy). The methods and compositions provided herein also allow for monitoring of a patient's response to neoadjuvant or adjuvant therapy comprising a PD-1 axis binding antagonist.

### I. Definitions

As used herein, “circulating tumor DNA” and “ctDNA” refer to tumor-derived DNA in the circulatory system that is not associated with cells. ctDNA is a type of cell-free DNA (cfDNA) that may originate from tumor cells or from circulating tumor cells (CTCs). ctDNA may be found, e.g., in the bloodstream of a patient, or in a biological sample (e.g., blood, serum, plasma, or urine) obtained from a patient. In some embodiments, ctDNA may include aberrant mutations (e.g., patient-specific variants) and/or methylation patterns.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED10680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additonal exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1LG1,” “CD274,” “B7-H,” and “PDL1.”

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

For the purposes herein, “atezolizumab” is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances), Proposed INN: List 112, Vol. 28, No. 4, published Jan. 16, 2015 (see page 485).

The term “cancer” refers to a disease caused by an uncontrolled division of abnormal cells in a part of the body. In one instance, the cancer is urothelial carcinoma. The cancer may be locally advanced or metastatic. In some instances, the cancer is locally advanced. In other instances, the cancer is metastatic. In some instances, the cancer may be unresectable (e.g., unresectable locally advanced or metastatic cancer).

As used herein, “urothelial carcinoma” and “UC” refer to a type of cancer that typically occurs in the urinary system, and includes muscle-invasive bladder cancer (MIBC) and muscle-invasive urinary tract urothelial cancer (UTUC). UC is also referred to in the art as transitional cell carcinoma (TCC).

As used herein, “tumor, node, and metastasis classification” and “TNM classification” refer to a cancer staging classification described in the American Joint Committee on Cancer (AJCC) Cancer Staging Manual, 7th Edition.

The term “ineligible for treatment with a platinum-based chemotherapy” or “unfit for treatment with a platinum-based chemotherapy” means that the subject is ineligible or unfit for treatment with a platinum-based chemotherapy, either in the attending clinician's judgment or according to standardized criteria for eligibility for platinum-based chemotherapy that are known in the art. For example, cisplatin ineligibility may be defined by any one of the following criteria: (i) impaired renal function (glomerular filtration rate (GFR)<60 mL/min); GFR may be assessed by direct measurement (i.e., creatinine clearance or ethyldediaminetetra-acetate) or, if not available, by calculation from serum/plasma creatinine (Cockcroft Gault formula); (ii) a hearing loss (measured by audiometry) of 25 dB at two contiguous frequencies; (iii) Grade 2 or greater peripheral neuropathy (i.e., sensory alteration or parasthesis including tingling); and (iv) ECOG Performance Status of 2.

As used herein, “treating” comprises effective cancer treatment with an effective amount of a therapeutic agent (e.g., a PD-1 axis binding antagonist (e.g., atezolizumab) or combination of therapeutic agents (e.g., a PD-1 axis antagonist and one or more additional therapeutic agents). Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the patient may be previously untreated or not have received prior systemic therapy), or second line or later treatment. In preferred examples, the treatment is adjuvant therapy. In other preferred examples, the treatment is neoadjuvant therapy.

Herein, an “effective amount” refers to the amount of a therapeutic agent (e.g., a PD-1 axis binding antagonist (e.g., atezolizumab) or a combination of therapeutic agents (e.g., a PD-1 axis antagonist and one or more additional therapeutic agents)), that achieves a therapeutic result. In some examples, the effective amount of a therapeutic agent or a combination of therapeutic agents is the amount of the agent or of the combination of agents that achieves a clinical endpoint of improved overall response rate (ORR), a complete response (CR), a pathological complete response (pCR), a partial response (PR), improved survival (e.g., disease-free survival (DFS), disease-specific survival (DSS), distant metastasis-free survival, progression-free survival (PFS) and/or overall survival (OS)), improved duration of response (DOR), improved time to deterioration of function and quality of life (QoL), and/or ctDNA clearance. Improvement (e.g., in terms of response rate (e.g., ORR, CR, and/or PR), survival (e.g., DFS, DSS, distant metastasis-free survival, PFS, and/or OS), DOR, improved time to deterioration of function and QoL, and/or ctDNA clearance) may be relative to a suitable reference, for example, observation or a reference treatment (e.g., treatment that does not include the PD-1 axis binding antagonist (e.g., treatment with placebo)). In some instances, improvement (e.g., in terms of response rate (e.g., ORR, CR, and/or PR), survival (e.g., DFS, DSS, distant metastasis-free survival, PFS, and/or OS), DOR, improved time to deterioration of function and QoL, and/or ctDNA clearance) may be relative to observation.

As used herein, “complete response” and “CR” refers to disappearance of all target lesions.

As used herein, “partial response” and “PR” refers to at least a 30% decrease in the sum of the longest diameters (SLD) of target lesions, taking as reference the baseline SLD prior to treatment.

As used herein, “overall response rate,” “objective response rate,” and “ORR” refer interchangeably to the sum of CR rate and PR rate.

As used herein, “disease-free survival” and “DFS” refer to the length of time after a primary treatment (e.g., surgical resection) that the patient survives without recurrence of the cancer. In some instances, DFS is defined as the time from randomization to the first occurrence of a DFS event, defined as any of the following: local (pelvic) recurrence of UC (including soft tissue and regional lymph nodes); urinary tract recurrence of UC (including all pathological stages and grades); distant metastasis of UC; or death from any cause.

As used herein, “disease-specific survival” and “DSS” refer to the length of time that the patient has not died from a specific disease (e.g., UC). In some instances, DSS may be defined as the time from randomization to death from UC (e.g., per investigator assessment of cause of death).

As used herein, “distant metastasis-free survival” refers to the length of time from either the date of diagnosis or the start of treatment that a patient is still alive and the cancer has not spread to other parts of the body. In some instances, distant metastasis-free survival is defined as the time from randomization to the diagnosis of distant (i.e., non-locoregional) metastases or death from any cause.

As used herein, “progression-free survival” and “PFS” refer to the length of time during and after treatment during which the cancer does not get worse. PFS may include the amount of time patients have experienced a CR or a PR, as well as the amount of time patients have experienced stable disease.

As used herein, “overall survival” and “OS” refer to the length of time from either the date of diagnosis or the start of treatment for a disease (e.g., cancer) that the patient is still alive. For example, OS may be defined as the time from randomization to death from any cause.

As used herein, the term “duration of response” and “DOR” refer to a length of time from documentation of a tumor response until disease progression or death from any cause, whichever occurs first.

As used herein, “time to deterioration of function and QoL” refers to the length of time from either the date of diagnosis or the start of treatment until deterioration of function or reduced quality of life. In some instances, time to deterioration of function and QoL is defined as the time from randomization to the date of a patient's first score decrease of a ≥10 points from baseline on the European Organisation for Research and Treatment of Cancer (EORTC) Quality of Life Questionnaire-Core 30 (QLQ-C30) physical function scale, role function scale, and the global health status (GHS)/QoL scale (separately).

The term “ctDNA clearance” as used herein refers to clearance of ctDNA in a patient or population of patients determined to be ctDNA-positive at baseline. In some instances, ctDNA clearance may be defined as the proportion of patients who are ctDNA-positive at baseline and ctDNA-negative at Cycle 3, Day 1 or Cycle 5, Day 1.

As used herein, the term “chemotherapeutic agent” refers to a compound useful in the treatment of cancer, such as urothelial carcinoma. Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)−2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², p³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

Chemotherapeutic agents also include “platinum-based” chemotherapeutic agents, which comprise an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin. In some instances, platinum-based chemotherapeutic agents (e.g., cisplatin or carboplatin) may be administered in combination with one or more additional chemotherapeutic agents, e.g., a nucleoside analog (e.g., gemcitabine).

A “platinum-based chemotherapy,” as used herein, refers to a chemotherapy regimen that includes a platinum-based chemotherapeutic agent. For example, a platinum-based chemotherapy may include a platinum-based chemotherapeutic agent (e.g., cisplatin or carboplatin), and, optionally, one or more additional chemotherapeutic agents, e.g., a nucleoside analog (e.g., gemcitabine).

The term “patient” refers to a human patient. For example, the patient may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447). Therefore, the C-terminal lysine (Lys447), or the C-terminal glycine (Gly446) and lysine (Lys447), of the Fc region may or may not be present. Amino acid sequences of heavy chains including an Fc region are denoted herein without the C-terminal lysine (Lys447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′)₂, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).
   Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2- CDR-H2(CDR-L2)-FR3- CDR-H3(CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of a PD-1 axis binding antagonist (e.g., atezolizumab) and an additional therapeutic agent. As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3 week cycle.

The term “detection” includes any means of detecting, including direct and indirect detection.

The term “biomarker” as used herein refers to an indicator, e.g., predictive, diagnostic, and/or prognostic, which can be detected in a sample, for example, ctDNA, PD-L1, or tissue tumor mutational burden (tTMB). In some aspects, the biomarker is the presence or level of ctDNA in a biological sample obtained from a patient. The biomarker may serve as an indicator of a particular subtype of a disease or disorder (e.g., cancer) characterized by certain molecular, pathological, histological, and/or clinical features. In some aspects, the biomarker may serve as an indicator of the likelihood of treatment benefit. Biomarkers include, but are not limited to, polynucleotides (e.g., DNA and/or RNA), polynucleotide copy number alterations (e.g., DNA copy numbers), polypeptides, polypeptide and polynucleotide modifications (e.g., post-translational modifications), carbohydrates, and/or glycolipid-based molecular markers.

The “amount” or “level” of a biomarker (e.g., ctDNA) associated with an increased clinical benefit to a patient is a detectable level in a biological sample. These can be measured by methods known to one skilled in the art and also disclosed herein. The presence, expression level, or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Increased expression,” “increased expression level,” “increased levels,” “elevated expression,” “elevated expression levels,” or “elevated levels” refers to an increased expression or increased levels of a biomarker in a patient relative to a control, such as an individual or individuals who are not suffering from the cancer (e.g., urothelial carcinoma) or an internal control (e.g., a housekeeping biomarker).

“Decreased expression,” “decreased expression level,” “decreased levels,” “reduced expression,” “reduced expression levels,” or “reduced levels” refers to a decreased expression or decreased levels of a biomarker in a patient relative to a control, such as an individual or individuals who are not suffering from the cancer (e.g., urothelial carcinoma) or an internal control (e.g., a housekeeping biomarker). In some embodiments, reduced expression is little or no expression.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

The term “sample,” as used herein, refers to a composition that is obtained or derived from a patient of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a patient of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid, saliva, sputum, tears, perspiration, mucus, tumor lysates, and tissue culture medium, tissue extracts such as homogenized tissue, tumor tissue, cellular extracts, and combinations thereof. In some aspects, the sample is a blood sample, a plasma sample, a serum sample, a urine sample, a cerebrospinal fluid (CSF) sample, a nasal swab sample, a saliva sample, a stool sample, or a vaginal fluid sample.

By “tissue sample” or “cell sample” is meant a collection of similar cells obtained from a tissue of a patient. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the patient. The tissue sample may also be primary or cultured cells or cell lines. Optionally, the tissue or cell sample is obtained from a disease tissue/organ. For instance, a “tumor sample” is a tissue sample obtained from a tumor (e.g., a liver tumor) or other cancerous tissue. The tissue sample may contain a mixed population of cell types (e.g., tumor cells and non-tumor cells, cancerous cells and non-cancerous cells). The tissue sample may contain compounds which are not naturally intermixed with the tissue in nature such as preservatives, anticoagulants, buffers, fixatives, nutrients, antibiotics, or the like.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+ T lymphocytes and/or CD4+ T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference level,” “reference sample,” “reference cell,” “reference tissue,” “control sample,” “control cell,” or “control tissue,” as used herein, refers to a level, sample, cell, tissue, or standard that is used for comparison purposes. In one example, a reference level, reference sample, reference cell, reference tissue, control sample, control cell, or control tissue is obtained from a healthy and/or non-diseased part of the body (e.g., tissue or cells) of the same patient. For example, the reference level, reference sample, reference cell, reference tissue, control sample, control cell, or control tissue may be healthy and/or non-diseased cells or tissue adjacent to the diseased cells or tissue (e.g., cells or tissue adjacent to a tumor). In another example, a reference sample is obtained from an untreated tissue and/or cell of the body of the same patient. In yet another example, a reference level, reference sample, reference cell, reference tissue, control sample, control cell, or control tissue is obtained from a healthy and/or non-diseased part of the body (e.g., tissues or cells) of an individual who is not the patient. In even another embodiment, a reference level, reference sample, reference cell, reference tissue, control sample, control cell, or control tissue is obtained from an untreated tissue and/or cell of the body of an individual who is not the patient.

For the purposes herein a “section” of a tissue sample is meant a single part or piece of a tissue sample, for example, a thin slice of tissue or cells cut from a tissue sample (e.g., a tumor sample). It is to be understood that multiple sections of tissue samples may be taken and subjected to analysis, provided that it is understood that the same section of tissue sample may be analyzed at both morphological and molecular levels, or analyzed with respect to polypeptides (e.g., by immunohistochemistry) and/or polynucleotides (e.g., by in situ hybridization).

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

The phrase “based on” when used herein means that the information about one or more biomarkers is used to inform a treatment decision, information provided on a package insert, or marketing/promotional guidance, and the like.

As used herein, the terms “mutational load,” “mutation load,” “mutational burden,” “tumor mutational burden score,” “TMB score,” “tissue tumor mutational burden score,” and “tTMB score” each of which may be used interchangeably, refer to the level (e.g., number) of an alteration (e.g., one or more alterations, e.g., one or more somatic alterations) per a pre-selected unit (e.g., per megabase) in a pre-determined set of genes (e.g., in the coding regions of the pre-determined set of genes) detected in a tumor tissue sample (e.g., a formalin-fixed and paraffin-embedded (FFPE) tumor sample, an archival tumor sample, a fresh tumor sample, or a frozen tumor sample). The tTMB score can be measured, for example, on a whole genome or exome basis, or on the basis of a subset of the genome or exome. In certain embodiments, the tTMB score measured on the basis of a subset of the genome or exome can be extrapolated to determine a whole genome or exome mutation load. In some embodiments, a tTMB score refers to the level of accumulated somatic mutations within a patient. The tTMB score may refer to accumulated somatic mutations in a patient with cancer (e.g., urothelial carcinoma). In some embodiments, a tTMB score refers to the accumulated mutations in the whole genome of a patient. In some embodiments, a tTMB score refers to the accumulated mutations within a particular tissue sample (e.g., tumor tissue sample biopsy, e.g., a urothelial carcinoma tumor sample) collected from a patient.

The terms “somatic variant,” “somatic mutation,” or “somatic alteration” refer to a genetic alteration occurring in the somatic tissues (e.g., cells outside the germline). Examples of genetic alterations include, but are not limited to, point mutations (e.g., the exchange of a single nucleotide for another (e.g., silent mutations, missense mutations, and nonsense mutations)), insertions and deletions (e.g., the addition and/or removal of one or more nucleotides (e.g., indels)), amplifications, gene duplications, copy number alterations (CNAs), rearrangements, and splice variants. The presence of particular mutations can be associated with disease states (e.g., cancer, e.g., urothelial carcinoma).

The term “patient-specific variant” refers to a variant (e.g., a somatic variant) present in a given patient's tumor. A patient-specific variant may be detected in ctDNA, e.g., using a personalized ctDNA multiplexed polymerase chain reaction (mPCR) approach. It is to be understood that a given patient-specific variant may be unique to the patient or may be present in the tumors of other individuals who are not the patient.

As used herein, the term “reference tTMB score” refers to a tTMB score against which another tTMB score is compared, e.g., to make a diagnostic, predictive, prognostic, and/or therapeutic determination. For example, the reference tTMB score may be a tTMB score in a reference sample, a reference population, and/or a pre-determined value. In some instances, the reference tTMB score is a cutoff value that significantly separates a first subset of patients who have been treated with a PD-1 axis binding antagonist therapy, in a reference population, and a second subset of patients who have not received a therapy or who have been treated with a non-PD-1 axis binding antagonist therapy, in the same reference population based on a significant difference between a patient's responsiveness in the absence of a therapy or to treatment with the PD-1 axis binding antagonist therapy, and a patient's responsiveness to treatment with the non-PD-1 axis binding antagonist therapy at or above the cutoff value and/or below the cutoff value. In some instances, the patient's responsiveness to treatment with a PD-1 axis binding antagonist therapy, is significantly improved relative to the patient's responsiveness in the absence of a therapy or to treatment with the non-PD-1 axis binding antagonist therapy at or above the cutoff value. In some instances, the patient's responsiveness in the absence or therapy or to treatment with the non-PD-L1 axis binding antagonist therapy is significantly improved relative to the patient's responsiveness to treatment with the PD-1 axis binding antagonist therapy, below the cutoff value.

It will be appreciated by one skilled in the art that the numerical value for the reference tTMB score may vary depending on the type of cancer, the methodology used to measure a tTMB score, and/or the statistical methods used to generate a tTMB score.

The term “equivalent TMB value” refers to a numerical value that corresponds to a tTMB score that can be calculated by dividing the count of somatic variants by the number of bases sequenced. In some instances, the whole exome is sequenced. In other instances, the number of sequenced bases is about 1.1 Mb (e.g., about 1.125 Mb), e.g., as assessed by the FOUNDATIONONE® panel). It is to be understood that, in general, the tTMB score is linearly related to the size of the genomic region sequenced. Such equivalent tTMB values indicate an equivalent degree of tumor mutational burden as compared to a tTMB score and can be used interchangeably in the methods described herein, for example, to predict response of a cancer patient to a PD-1 axis binding antagonist (e.g., an anti-PD-L1 antibody, e.g., atezolizumab). As an example, in some instances, an equivalent tTMB value is a normalized tTMB value that can be calculated by dividing the count of somatic variants (e.g., somatic mutations) by the number of bases sequenced. For example, an equivalent tTMB value can be represented as the number of somatic mutations counted over a defined number of sequenced bases (e.g., about 1.1 Mb (e.g., about 1.125 Mb), e.g., as assessed by the FOUNDATIONONE® panel). For example, a tTMB score of about 25 (as determined as the number of somatic mutations counted over about 1.1 Mb) corresponds to an equivalent tTMB value of about 23 mutations/Mb. It is to be understood that tTMB scores as described herein (e.g., TMB scores represented as the number of somatic mutations counted over a defined number of sequenced bases (e.g., about 1.1 Mb (e.g., about 1.125 Mb), e.g., as assessed by the FOUNDATIONONE® panel)) encompass equivalent tTMB values obtained using different methodologies (e.g., whole-exome sequencing or whole-genome sequencing). As an example, for a whole-exome panel, the target region may be approximately 50 Mb, and a sample with about 500 somatic mutations detected is an equivalent tTMB value to a tTMB score of about 10 mutations/Mb. In some instances, a tTMB score determined as the number of somatic mutations counted over a defined number of sequenced bases (e.g., about 1.1 Mb (e.g., about 1.125 Mb), e.g., as assessed by the FOUNDATIONONE® panel) in a subset of the genome or exome (e.g., a predetermined set of genes) deviates by less than about 30% (e.g., less than about 30%, about 25%, about 20%, about 15%, about 10%, about 5%, about 4%, about 3%, about 2%, about 1%, or less) from a tTMB score determined by whole-exome sequencing. See, e.g., Chalmers et al. Genome Medicine 9:34, 2017.

### II. Therapeutic Methods and Compositions for Urothelial Carcinoma

Provided herein are methods, compositions, and uses for neoadjuvant therapy and/or adjuvant therapy of urothelial carcinoma (e.g., MIUC) in a patient in need thereof. The methods, compositions, and uses may involve administration of a PD-1 axis binding antagonist (e.g., an anti-PD-L1 antibody such as atezolizumab) to a patient based on the presence and/or level of ctDNA in a biological sample obtained from the patient. In some instances, the methods, compositions, and uses may involve determining whether ctDNA is present or absent in a biological sample obtained from the patient (in other words, whether the biological sample is ctDNA-positive or ctDNA-negative). In other instances, the methods, compositions, and uses may involve determining a level of ctDNA in a biological sample, which may be compared to a reference ctDNA level.

In one aspect, provided herein is a method of treating urothelial carcinoma (e.g., MIUC) in a patient in need thereof, the method comprising administering to the patient an effective amount of a treatment regimen comprising a PD-1 axis binding antagonist, wherein the treatment regimen is an adjuvant therapy, and wherein the patient has been identified as likely to benefit from the treatment regimen based on the presence of ctDNA in a biological sample obtained from the patient.

In another aspect, provided herein is a method of treating urothelial carcinoma (e.g., MIUC) in a patient in need thereof, the method comprising: (a) determining whether ctDNA is present in a biological sample obtained from the patient, wherein the presence of ctDNA in the biological sample indicates that the patient is likely to benefit from a treatment regimen comprising a PD-1 axis binding antagonist; and (b) administering an effective amount of a treatment regimen comprising a PD-1 axis binding antagonist to the patient based on the presence of ctDNA in the biological sample, wherein the treatment regimen is an adjuvant therapy.

In another aspect, provided herein is a PD-1 axis binding antagonist, or a pharmaceutical composition comprising a PD-1 axis binding antagonist, for use in treatment of urothelial carcinoma (e.g., MIUC) in a patient in need thereof, wherein the treatment comprises administration of an effective amount of a treatment regimen comprising a PD-1 axis binding antagonist, wherein the treatment regimen is an adjuvant therapy, and wherein the patient has been identified as likely to benefit from the treatment regimen based on the presence of ctDNA in a biological sample obtained from the patient.

In another aspect, provided herein is a PD-1 axis binding antagonist, or a pharmaceutical composition comprising a PD-1 axis binding antagonist, for use in treatment of urothelial carcinoma (e.g., MIUC) in a patient in need thereof, the treatment comprising: (a) determining whether ctDNA is present in a biological sample obtained from the patient, wherein the presence of ctDNA in the biological sample indicates that the patient is likely to benefit from a treatment regimen comprising a PD-1 axis binding antagonist; and (b) administering an effective amount of a treatment regimen comprising a PD-1 axis binding antagonist to the patient based on the presence of ctDNA in the biological sample, wherein the treatment regimen is an adjuvant therapy.

In another aspect, provided herein is a method of treating urothelial carcinoma (e.g., MIUC) in a patient in need thereof, the method comprising administering to the patient an effective amount of a treatment regimen comprising a PD-1 axis binding antagonist, wherein the treatment regimen is an adjuvant therapy, and wherein a level of ctDNA in a biological sample obtained from the patient that is at or above a reference level for ctDNA indicates that the patient is likely to benefit from a treatment regimen comprising a PD-1 axis binding antagonist.

In another aspect, provided herein is a method of treating urothelial carcinoma (e.g., MIUC) in a patient in need thereof, the method comprising: (a) determining the level of ctDNA in a biological sample obtained from the patient, wherein a level of ctDNA in the biological sample that is at or above a reference level for ctDNA indicates that the patient is likely to benefit from a treatment regimen comprising a PD-1 axis binding antagonist; and (b) administering an effective amount of a treatment regimen comprising a PD-1 axis binding antagonist to the patient based on the level of ctDNA in the biological sample, wherein the treatment regimen is an adjuvant therapy.

In another aspect, provided herein is a PD-1 axis binding antagonist, or a pharmaceutical composition comprising a PD-1 axis binding antagonist, for use in treatment of urothelial carcinoma (e.g., MIUC) in a patient in need thereof, wherein the treatment comprises administration of an effective amount of a treatment regimen comprising a PD-1 axis binding antagonist, wherein the treatment regimen is an adjuvant therapy, and wherein a level of ctDNA in a biological sample obtained from the patient that is at or above a reference level for ctDNA indicates that the patient is likely to benefit from a treatment regimen comprising a PD-1 axis binding antagonist.

In another aspect, provided herein is a PD-1 axis binding antagonist, or a pharmaceutical composition comprising a PD-1 axis binding antagonist, for use in treatment of urothelial carcinoma (e.g., MIUC) in a patient in need thereof, the treatment comprising: (a) determining the level of ctDNA in a biological sample obtained from the patient, wherein a level of ctDNA in the biological sample that is at or above a reference level for ctDNA indicates that the patient is likely to benefit from a treatment regimen comprising a PD-1 axis binding antagonist; and (b) administering an effective amount of a treatment regimen comprising a PD-1 axis binding antagonist to the patient based on the level of ctDNA in the biological sample, wherein the treatment regimen is an adjuvant therapy.

In another aspect, provided herein is a method of identifying a patient having a urothelial carcinoma (e.g., MIUC) who may benefit from a treatment regimen comprising a PD-1 axis binding antagonist, the method comprising determining whether ctDNA is present in a biological sample obtained from the patient, wherein the presence of ctDNA in the biological sample identifies the patient as one who may benefit from treatment with a treatment regimen comprising a PD-1 axis binding antagonist, wherein the treatment regimen is an adjuvant therapy. In some instances, the method further comprises administering an effective amount of a treatment regimen comprising a PD-1 axis binding antagonist to the patient.

In another aspect, provided herein is a method of identifying a patient having a urothelial carcinoma (e.g., MIUC) who may benefit from a treatment regimen comprising a PD-1 axis binding antagonist, the method comprising determining the level of ctDNA in a biological sample obtained from the patient, wherein a level of ctDNA in the biological sample that is at or above a reference level for ctDNA indicates that the patient is likely to benefit from a treatment regimen comprising a PD-1 axis binding antagonist, wherein the treatment regimen is an adjuvant therapy. In some instances, the method further comprises administering an effective amount of a treatment regimen comprising a PD-1 axis binding antagonist to the patient.

In another aspect, provided herein is a method for selecting a therapy for a patient having a urothelial carcinoma (e.g., MIUC), the method comprising (a) determining whether ctDNA is present in a biological sample obtained from the patient, wherein the presence of ctDNA in the biological sample indicates that the patient is likely to benefit from a treatment regimen comprising a PD-1 axis binding antagonist; and (b) selecting a treatment regimen comprising a PD-1 axis binding antagonist based on the presence of ctDNA in the biological sample, wherein the treatment regimen is an adjuvant therapy. In some instances, the method further comprises administering an effective amount of a treatment regimen comprising a PD-1 axis binding antagonist to the patient.

In another aspect, provided herein is a method for selecting a therapy for a patient having a urothelial carcinoma (e.g., MIUC), the method comprising (a) determining the level of ctDNA in a biological sample obtained from the patient, wherein a level of ctDNA in the biological sample that is at or above a reference level for ctDNA indicates that the patient is likely to benefit from a treatment regimen comprising a PD-1 axis binding antagonist; and (b) selecting a treatment regimen comprising a PD-1 axis binding antagonist based on the presence of ctDNA in the biological sample, wherein the treatment regimen is an adjuvant therapy. In some instances, the method further comprises administering an effective amount of a treatment regimen comprising a PD-1 axis binding antagonist to the patient.

In some instances, the biological sample is obtained prior to or concurrently with administration of a first dose of the treatment regimen. In some instances, the biological sample is obtained on cycle 1, day 1 (C1D1) of the treatment regimen. In some instances, the biological sample is obtained within about 60 weeks (e.g., within about 60 weeks, about 55 weeks, about 50 weeks, about 45 weeks, about 40 weeks, about 35 weeks, about 30 weeks, about 25 weeks, about 20 weeks, about 19 weeks, about 18 weeks, about 17 weeks, about 16 weeks, about 15 weeks, about 14 weeks, about 13 weeks, about 12 weeks, about 11 weeks, about 10 weeks, about 9 weeks, about 8 weeks, about 7 weeks, about 6 weeks, about 5 weeks, about 4 weeks, about 3 weeks, about 2 weeks, or about 1 week) from surgical resection.

In some instances, the biological sample is obtained within about 30 weeks from surgical resection. In some instances, the biological sample is obtained within about 20 weeks from surgical resection. In some instances, the biological sample is obtained about 2 to about 20 weeks (e.g., about 2 to about 20 weeks, about 2 to about 19 weeks, about 2 to about 18 weeks, about 2 to about 17 weeks, about 2 to about 16 weeks, about 2 to about 15 weeks, about 2 to about 14 weeks, about 2 to about 13 weeks, about 2 to about 12 weeks, about 2 to about 11 weeks, about 2 to about 10 weeks, about 2 to about 9 weeks, about 2 to about 8 weeks, about 2 to about 7 weeks, about 2 to about 6 weeks, about 2 to about 5 weeks, about 2 to about 4 weeks, about 2 to about 3 weeks, about 4 to about 20 weeks, about 4 to about 19 weeks, about 4 to about 18 weeks, about 4 to about 17 weeks, about 4 to about 16 weeks, about 4 to about 15 weeks, about 4 to about 14 weeks, about 4 to about 13 weeks, about 4 to about 12 weeks, about 4 to about 11 weeks, about 4 to about 10 weeks, about 4 to about 9 weeks, about 4 to about 8 weeks, about 4 to about 7 weeks, about 4 to about 6 weeks, about 4 to about 5 weeks, about 6 to about 20 weeks, about 6 to about 19 weeks, about 6 to about 18 weeks, about 6 to about 17 weeks, about 6 to about 16 weeks, about 6 to about 15 weeks, about 6 to about 14 weeks, about 6 to about 13 weeks, about 6 to about 12 weeks, about 6 to about 11 weeks, about 6 to about 10 weeks, about 6 to about 9 weeks, about 6 to about 8 weeks, about 6 to about 7 weeks, about 8 to about 20 weeks, about 8 to about 19 weeks, about 8 to about 18 weeks, about 8 to about 17 weeks, about 6 to about 16 weeks, about 6 to about 15 weeks, about 6 to about 14 weeks, about 8 to about 13 weeks, about 8 to about 12 weeks, about 8 to about 11 weeks, about 8 to about 10 weeks, about 8 to about 9 weeks, about 10 to about 20 weeks, about 10 to about 19 weeks, about 10 to about 18 weeks, about 10 to about 17 weeks, about 10 to about 16 weeks, about 10 to about 15 weeks, about 10 to about 14 weeks, about 10 to about 13 weeks, about 10 to about 12 weeks, about 10 to about 11 weeks, about 12 to about 20 weeks, about 12 to about 19 weeks, about 12 to about 18 weeks, about 12 to about 17 weeks, about 12 to about 16 weeks, about 12 to about 15 weeks, about 12 to about 14 weeks, about 12 to about 13 weeks, about 14 to about 20 weeks, about 14 to about 19 weeks, about 14 to about 18 weeks, about 14 to about 17 weeks, about 14 to about 16 weeks, about 14 to about 15 weeks, about 16 to about 20 weeks, about 16 to about 19 weeks, about 16 to about 18 weeks, about 16 to about 17 weeks, about 18 to about 20 weeks, or about 18 to about 19 weeks) after surgical resection.

It is to be understood that ctDNA may be detected in any suitable biological sample. In some instances, the biological sample is a blood sample, a plasma sample, a serum sample, a urine sample, a CSF sample, a nasal swab sample, a saliva sample, a stool sample, or a vaginal fluid sample. In some instances, the biological sample is a blood sample, a plasma sample, or a serum sample. In some instances, the biological sample is a plasma sample.

In another aspect, provided herein is a method of monitoring the response of a patient having a urothelial carcinoma (e.g., MIUC) who has been administered at least a first dose of a treatment regimen comprising a PD-1 axis binding antagonist, wherein the treatment regimen is a neoadjuvant therapy or an adjuvant therapy, and wherein ctDNA was present in a biological sample obtained from the patient prior to or concurrently with the first dose of the treatment regimen, the method comprising determining whether ctDNA is present in a biological sample obtained from the patient at a time point following administration of the first dose of the treatment regimen, thereby monitoring the response of the patient.

In some instances, an absence of ctDNA in the biological sample obtained from the patient at a time point following administration of the first dose of the treatment regimen indicates that the patient is responding to the treatment regimen. In some embodiments, the treatment regimen is a neoadjuvant therapy. In other embodiments, the treatment regimen is an adjuvant therapy.

In another aspect, provided herein is a method of monitoring the response of a patient having a urothelial carcinoma (e.g., MIUC) who has been administered at least a first dose of a treatment regimen comprising a PD-1 axis binding antagonist, wherein the treatment regimen is a neoadjuvant therapy or an adjuvant therapy, and wherein a level of ctDNA was present in a biological sample obtained from the patient prior to or concurrently with the first dose of the treatment regimen, the method comprising determining the level of ctDNA in a biological sample obtained from the patient at a time point following administration of the first dose of the treatment regimen, thereby monitoring the response of the patient. In some instances, a decrease in the level of ctDNA in the biological sample obtained from the patient at a time point following administration of the first dose of the treatment regimen relative to the level of ctDNA in the biological sample obtained from the patient prior to or concurrently with the first dose of the treatment regimen indicates that the patient is responding to the treatment regimen. In some embodiments, the treatment regimen is a neoadjuvant therapy. In other embodiments, the treatment regimen is an adjuvant therapy.

In another aspect, provided herein is a PD-1 axis binding antagonist, or a pharmaceutical composition comprising a PD-1 axis binding antagonist, for use in treatment of a patient having a urothelial carcinoma (e.g., MIUC) who has been administered at least a first dose of a treatment regimen comprising a PD-1 axis binding antagonist, wherein the treatment regimen is a neoadjuvant therapy or an adjuvant therapy, and wherein ctDNA was present in a biological sample obtained from the patient prior to or concurrently with the first dose of the treatment regimen. In some embodiments, the treatment regimen is a neoadjuvant therapy. In other embodiments, the treatment regimen is an adjuvant therapy.

In another aspect, provided herein is a method of identifying a patient having a urothelial carcinoma (e.g., MIUC) who may benefit from a treatment regimen comprising a PD-1 axis binding antagonist, wherein the treatment regimen is a neoadjuvant therapy or an adjuvant therapy and the patient has been administered at least a first dose of the treatment regimen, and wherein ctDNA was present in a biological sample obtained from the patient prior to or concurrently with the first dose of the treatment regimen, the method comprising: determining whether ctDNA is present in a biological sample obtained from the patient at a time point following administration of the first dose of the treatment regimen, wherein an absence of ctDNA in the biological sample at the time point following administration of the treatment regimen identifies the patient as one who may benefit from treatment with a treatment regimen comprising a PD-1 axis binding antagonist. In some embodiments, the treatment regimen is a neoadjuvant therapy. In other embodiments, the treatment regimen is an adjuvant therapy.

In another aspect, provided herein is a method of identifying a patient having a urothelial carcinoma (e.g., MIUC) who may benefit from a treatment regimen comprising a PD-1 axis binding antagonist, wherein the treatment regimen is a neoadjuvant therapy or an adjuvant therapy and the patient has been administered at least a first dose of the treatment regimen, and wherein a level of ctDNA was present in a biological sample obtained from the patient prior to or concurrently with the first dose of the treatment regimen, the method comprising: determining the level of ctDNA in a biological sample obtained from the patient at a time point following administration of the first dose of the treatment regimen, wherein a decrease in the level of ctDNA in the biological sample at the time point following administration of the treatment regimen relative to the level of ctDNA in the biological sample obtained from the patient prior to or concurrently with the first dose of the treatment regimen identifies the patient as one who may benefit from treatment with a treatment regimen comprising a PD-1 axis binding antagonist. In some embodiments, the treatment regimen is a neoadjuvant therapy. In other embodiments, the treatment regimen is an adjuvant therapy.

Any suitable time point following administration of the first dose of the treatment regimen may be used. For example, in some instances, the time point following administration of the first dose of the treatment regimen is on cycle 2, day 1 (C2D1, cycle 3, day 1 (C3D1), cycle 4, day 1 (C4D1), cycle 5, day 1 (C5D1), cycle 6, day 1 (C6D1), cycle 7, day 1 (C7D1), cycle 8, day 1 (C8D1), cycle 9, day 1 (C9D1), cycle 10, day 1 (C10D1), cycle 11, day 1 (C11D1), cycle 12, day 1 (C12D1), or on subsequent cycles of the treatment regimen. However, it is to be understood that the biological sample obtained at the time point following administration of the treatment regimen may be obtained on any day of the treatment cycle (e.g., any day on a 14-day cycle, any day on a 21-day cycle, or any day on a 28-day cycle).

In some instances, the biological sample obtained from the patient prior to or concurrently with a first dose of the treatment regimen and/or the biological sample obtained from the patient at a time point following administration of the first dose of the treatment regimen is a blood sample, a plasma sample, a serum sample, a urine sample, a CSF sample, a nasal swab sample, a saliva sample, a stool sample, or a vaginal fluid sample. For example, in some instances, the biological sample obtained from the patient prior to or concurrently with a first dose of the treatment regimen and/or the biological sample obtained from the patient at a time point following administration of the first dose of the treatment regimen is a plasma sample.

In some instances, the benefit is in terms of improved disease-free survival (DFS), improved overall survival (OS), improved disease-specific survival, or improved distant metastasis-free survival. In some instances, the benefit is in terms of improved DFS. In some instances, the benefit is in terms of improved OS. In some instances, improvement is relative to observation or relative to adjuvant therapy with a placebo.

The presence and/or level of ctDNA in a biological sample may be determined using any suitable approach, e.g., any approach known in the art or described in Section V below. For example, in some instances, the presence and/or level of ctDNA is determined by a polymerase chain reaction (PCR)-based approach, a hybridization capture-based approach, a methylation-based approach, or a fragmentomics approach.

In some instances, the presence and/or level of ctDNA is determined by a personalized ctDNA multiplexed polymerase chain reaction (mPCR) approach. In some instances, the personalized ctDNA mPCR approach comprises: (a) (i) sequencing DNA obtained from a tumor sample obtained from the patient to produce tumor sequence reads; and (ii) sequencing DNA obtained from a normal tissue sample (e.g., buffy coat) obtained from the patient to produce normal sequence reads; (b) identifying one or more patient-specific variants by calling somatic variants identified from the tumor sequence reads and excluding germline variants and/or clonal hematopoiesis of indeterminate potential (CHIP) variants, wherein the germline variants or CHIP variants are identified from the normal sequence reads or from a publicly available database; (c) designing an mPCR assay for the patient that detects a set of patient-specific variants; and (d) analyzing a biological sample obtained from the patient using the mPCR assay to determine whether ctDNA is present in the biological sample. In some instances, the sequencing is WES or WGS. In some instances, the sequencing is WES. In some instances, the patient-specific variants are single nucleotide variants (SNVs) or short indels (insertion or deletion of bases). In some instances, the set of patient-specific variants comprises at least 1 patient-specific variant. In some instances, the set of patient-specific variants comprises at least 2 patient-specific variants. In some instances, the set of patient-specific variants comprises at least 8 patient-specific variants. In some instances, the set of patient-specific variants comprises 2 to 200 patient-specific variants. In some instances, the set of patient-specific variants comprises 8 to 50 patient-specific variants. In some instances, the set of patient-specific variants comprises 8 to 32 patient-specific variants. In some instances, the set of patient-specific variants comprises 16 patient-specific variants. In some instances, analyzing the biological sample obtained from the patient using the mPCR assay comprises sequencing amplicons produced by the mPCR assay to identify patient-specific variants in the biological sample. In some instances, the personalized ctDNA mPCR approach is a SIGNATERA® ctDNA test or an ArcherDx Personalized Cancer Monitoring (PCM™) test. In some instances, the presence of at least one patient-specific variant in the biological sample identifies the presence of ctDNA in the biological sample. In some instances, the presence of two patient-specific variants in the biological sample identifies the presence of ctDNA in the biological sample.

In some instances, about 2 to about 200 patient-specific variants are detected in the biological sample, e.g., about 2 to about 200, about 2 to about 175, about 2 to about 150, about 2 to about 125, about 2 to about 100, about 2 to about 75, about 2 to about 50, about 2 to about 48, about 2 to 46, about 2 to 44, about 2 to 42, about 2 to 40, about 2 to 38, about 2 to 36, about 2 to 34, about 2 to about 32, about 2 to about 30, about 2 to about 28, about 2 to about 26, about 2 to about 24, about 2 to about 22, about 2 to about 20, about 2 to about 18, about 2 to about 16, about 2 to about 14, about 2 to about 12, about 2 to about 10, about 2 to about 8, about 2 to about 6, about 2 to about 4, about 4 to about 32, about 4 to about 30, about 4 to about 28, about 4 to about 26, about 4 to about 24, about 4 to about 22, about 4 to about 20, about 4 to about 18, about 4 to about 16, about 4 to about 14, about 4 to about 12, about 4 to about 10, about 4 to about 8, about 4 to about 6, about 6 to about 32, about 6 to about 30, about 6 to about 28, about 6 to about 26, about 6 to about 24, about 6 to about 22, about 6 to about 20, about 6 to about 18, about 6 to about 16, about 6 to about 14, about 6 to about 12, about 6 to about 10, about 6 to about 8, about 8 to about 32, about 8 to about 30, about 8 to about 28, about 8 to about 26, about 8 to about 24, about 8 to about 22, about 8 to about 20, about 8 to about 18, about 8 to about 16, about 8 to about 14, about 8 to about 12, about 8 to about 10, about 10 to about 32, about 10 to about 30, about 10 to about 28, about 10 to about 26, about 10 to about 24, about 10 to about 22, about 10 to about 20, about 10 to about 18, about 10 to about 16, about 10 to about 14, about 10 to about 12, about 12 to about 32, about 12 to about 30, about 12 to about 28, about 12 to about 26, about 12 to about 24, about 12 to about 22, about 12 to about 20, about 12 to about 18, about 12 to about 16, about 12 to about 14, about 14 to about 32, about 14 to about 30, about 14 to about 28, about 14 to about 26, about 14 to about 24, about 14 to about 22, about 14 to about 20, about 14 to about 18, about 14 to about 16, about 16 to about 32, about 16 to about 30, about 16 to about 28, about 16 to about 26, about 16 to about 24, about 16 to about 22, about 16 to about 20, about 16 to about 18, about 18 to about 32, about 18 to about 30, about 18 to about 28, about 18 to about 26, about 18 to about 24, about 18 to about 22, about 18 to about 20, about 20 to about 32, about 20 to about 30, about 20 to about 28, about 20 to about 26, about 20 to about 24, about 20 to about 22, about 22 to about 32, about 22 to about 30, about 22 to about 28, about 22 to about 26, about 22 to about 24, about 24 to about 32, about 24 to about 30, about 24 to about 28, about 24 to about 26, about 26 to about 32, about 26 to about 30, about 26 to about 28, about 28 to about 32, about 28 to about 30, or about 30 to about 32 patient-specific variants. In some instances, about 2 to about 16 patient-specific variants are detected in the biological sample.

In some instances, the mean allele frequency for a given patient-specific variant in the biological sample is about 0.0001% to about 99%, e.g., about 0.0001%, about 0.0002%, about 0.0003%, about 0.0004%, about 0.0005%, about 0.0006%, about 0.0007%, about 0.0008%, about 0.0009%, about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%. In some instances, the mean allele frequency for a given patient-specific variant in the biological sample is about 0.001% to about 99%.

The biological sample may have any suitable volume. For example, in some instances, the biological sample has a volume of about 0.02 mL to about 80 mL (e.g., about 0.02 mL, about 0.3 mL, about 0.4 mL, about 0.5 mL, about 0.6 mL, about 0.7 mL, about 0.8 mL, about 0.9 mL, about 1 mL, about 2 mL, about 3 mL, about 4 mL, about 5 mL, about 6 mL, about 7 mL, about 8 mL, about 9 mL, about 10 mL, about 12 mL, about 14 mL, about 16 mL, about 18 mL, about 20 mL, about 22 mL, about 24 mL, about 26 mL, about 28 mL, about 30 mL, about 32 mL, about 34 mL, about 36 mL, about 38 mL, about 40 mL, about 45 mL, about 50 mL, about 55 mL, about 60 mL, about 65 mL, about 70 mL, about 75 mL, or about 80 mL).

For example, in some instances, the biological sample has a volume of about 1 mL to about 20 mL (e.g., about 2 mL to about 20 mL, about 2 mL to about 18 mL, about 2 mL to about 16 mL, about 2 mL to about 14 mL, about 2 mL to about 12 mL, about 2 mL to about 10 mL, about 2 mL to about 8 mL, about 2 mL to about 6 mL, about 2 mL to about 4 mL, about 4 mL to about 20 mL, about 4 mL to about 18 mL, about 4 mL to about 16 mL, about 4 mL to about 14 mL, about 4 mL to about 12 mL, about 4 mL to about 10 mL, about 4 mL to about 8 mL, about 4 mL to about 6 mL, about 6 mL to about 20 mL, about 6 mL to about 18 mL, about 6 mL to about 16 mL, about 6 mL to about 14 mL, about 6 mL to about 12 mL, about 6 mL to about 10 mL, about 6 mL to about 8 mL, about 8 mL to about 20 mL, about 8 mL to about 18 mL, about 8 mL to about 16 mL, about 8 mL to about 14 mL, about 8 mL to about 12 mL, about 8 mL to about 10 mL, about 10 mL to about 20 mL, about 10 mL to about 18 mL, about 10 mL to about 16 mL, about 10 mL to about 14 mL, about 10 mL to about 12 mL, about 12 mL to about 20 mL, about 12 mL to about 18 mL, about 12 mL to about 16 mL, about 12 mL to about 14 mL, about 14 mL to about 20 mL, about 14 mL to about 18 mL, about 14 mL to about 16 mL, about 16 mL to about 20 mL, about 16 mL to about 18 mL, or about 18 mL to about 20 mL). In some instances, the biological sample has a volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, or about 20 mL. In some instances, the biological sample has a volume of about 2 to about 10 mL. In some instances, the biological sample has a volume of about 2 to about 8 mL.

The biological sample may contain any suitable amount of cfDNA (e.g., ctDNA). For example, the biological sample may contain about 2 ng to about 200 ng (e.g., about 2 ng, about 5 ng, about 10 ng, about 15 ng, about 20 ng, about 25 ng, about 30 ng, about 35 ng, about 40 ng, about 45 ng, about 50 ng, about 55 ng, about 60 ng, about 65 ng, about 70 ng, about 80 ng, about 85 ng, about 90 ng, about 95 ng, about 100 ng, about 105 ng, about 110 ng, about 115 ng, about 120 ng, about 125 ng, about 130 ng, about 135 ng, about 140 ng, about 145 ng, about 150 ng, about 155 ng, about 160 ng, about 165 ng, about 170 ng, about 175 ng, about 180 ng, about 185 ng, about 190 ng, about 195 ng, or about 200 ng) of cfDNA (e.g., ctDNA). In some instances, the biological sample may contain about 10 to about 70 ng of cfDNA (e.g., ctDNA).

In instances where a level of ctDNA is determined, the level of ctDNA may be expressed, e.g., as the variant allele frequency (VAF) or in terms of mutations/mL.

In instances where a level of ctDNA is determined, any suitable reference level for ctDNA may be used. For example, the reference level for ctDNA may be (1) the level of ctDNA in a biological sample obtained from the patient prior to or concurrently with administration of a treatment regimen comprising a PD-1 axis binding antagonist; (2) the level of ctDNA from a reference population; (3) a pre-assigned level for ctDNA; or (4) the level of ctDNA in a biological sample obtained from the patient at a second time point prior to or after the first time point.

In some instances, the urothelial carcinoma is MIUC. In some instances, the MIUC is muscle-invasive bladder cancer (MIBC) or muscle-invasive urinary tract urothelial cancer (muscle-invasive UTUC). In some instances, the MIUC is histologically confirmed and/or wherein the patient has an Eastern Cooperative Oncology Group (ECOG) Performance Status of less than or equal to 2.

In some instances, the patient has previously been treated with neoadjuvant chemotherapy. In some instances, the patient's MIUC is ypT2-4a or ypN+ and M0 at surgical resection. In some instances, the patient has not received prior neoadjuvant chemotherapy.

In other instances, the patient is cisplatin-ineligible or has refused cisplatin-based adjuvant chemotherapy. In some instances, the patient's MIUC is pT3-4a or pN+ and M0 at surgical resection.

In some instances, the patient has undergone surgical resection with lymph node dissection. In some instances, the surgical resection is cystectomy or nephroureterectomy.

In some instances, the patient has no evidence of residual disease or metastasis as assessed by postoperative radiologic imaging.

In some instances, a tumor sample obtained from the patient has been determined to have a tissue tumor mutational burden (tTMB) score that is at or above a reference tTMB score. In some instances, the reference tTMB score is a pre-assigned tTMB score. In some instances, the pre-assigned tTMB score is between about 8 and about 30 mut/Mb. In some instances, the pre-assigned tTMB score is about 10 mutations per megabase (mut/Mb).

In some instances, the tumor sample is from surgical resection.

In some instances, the patient has an increased expression level of one or more genes selected from PD-L1, IFNG, and CXCL9 relative to a reference expression level of the one or more genes in a biological sample obtained from the patient.

In some instances, the patient has an increased expression level of two or more genes selected from PD-L1, IFNG, and CXCL9 relative to a reference expression level of the two or more genes in the biological sample obtained from the patient. For example, in some instances, the patient may have an increased expression level of PD-L1 and IFNG, PD-L1 and CXCL9, or IFNG and CXCL9, relative to a reference expression level of the two or more genes.

In some instances, the patient has an increased expression level of PD-L1, IFNG, and CXCL9 relative to a reference expression level of PD-L1, IFNG, and CXCL9 in the biological sample obtained from the patient.

In some instances involving determination of the expression level of PD-L1, IFNG, and/or CXCL9, an expression level above a reference expression level, or an elevated or increased expression or number, may refer to an overall increase of about any of 10%, 20%, 30%, 40%, 50%, 60%, 70%, 80%, 90%, 95%, 96%, 97%, 98%, 99% or greater, in the level or number of a biomarker (e.g., protein, nucleic acid (e.g., gene or mRNA), or cell), detected by methods such as those described herein and/or known in the art, as compared to a reference expression level, reference sample, reference cell, reference tissue, control sample, control cell, or control tissue. In certain embodiments, the elevated expression or number refers to the increase in expression level/amount of a biomarker (e.g., one or more of PD-L1, IFNG, and/or CXCL9) in the sample wherein the increase is at least about any of 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, 3×, 3.5×, 4×, 4.5×, 5×, 6×, 7×, 8×, 9×, 10×, 15×, 20×, 30×, 40×, 50×, 100×, 500×, or 1000× the expression level/amount of the respective biomarker in a reference expression level, reference sample, reference cell, reference tissue, control sample, control cell, or control tissue. In some embodiments, elevated expression or number refers to an overall increase in expression level/amount of a biomarker (e.g., PD-L1, IFNG, and/or CXCL9) of greater than about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.5-fold, about 4-fold, about 4.5-fold, about 5-fold, about 6-fold, about 7-fold, about 8-fold, about 9-fold, about 10-fold, about 15-fold, about 20-fold, about 30-fold, about 40-fold, about 50-fold, about 100-fold, about 500-fold, about 1,000-fold or greater as compared to a reference expression level, reference sample, reference cell, reference tissue, control sample, control cell, control tissue, or internal control (e.g., housekeeping gene).

In some instances, the expression level of PD-L1, IFNG, and/or CXCL9 is an mRNA expression level. In other instances, the expression level of PD-L1, IFNG, and/or CXCL9 may be a protein expression level.

In some instances, the expression level of a pan-F-TBRS signature may be determined in a a biological sample obtained from the patient. The expression level of a pan-F-TBRS signature may be determined, e.g., as described in U.S. Patent Application Publication No. 2020/0263261, which is incorporated herein by reference in its entirety. In other examples, the expression level of any signature described in U.S. Patent Application Publication No. 2020/0263261 may be determined, including the 22-gene (e.g., TGFB1, TGFBR2, ACTA2, ACTG2, ADAM12, ADAM19, COMP, CNN1, COL4A1, CTGF, CTPS1, FAM101B, FSTL3, HSPB1, IGFBP3, PXDC1, SEMA7A, SH3PXD2A, TAGLN, TGFBI, TNS1, and/or TPM1) or 6-gene (ACTA2, ADAM19, COMP, CTGF, TGFB1, and/or TGFBR2) signatures, including any combination of genes described in U.S. Patent Application Publication No. 2020/0263261. In another example, the signature may be a pan-F-TBRS signature including one or more genes selected from ACTA2, ACTG2, TAGLN, TNS1, CNN1, TPM1, CTGF, PXDC1, ADAM12, FSTL3, TGFBI, and ADAM19.

In some instances, the patient has a decreased expression level of one or more pan-F-TBRS genes selected from ACTA2, ACTG2, TAGLN, TNS1, CNN1, TPM1, CTGF, PXDC1, ADAM12, FSTL3, TGFBI, and ADAM19 relative to a reference expression level of the one or more pan-F-TBRS genes in a biological sample obtained from the patient.

In some instances, the patient has a decreased expression level of at least two, at least three, at least four, at least five, at least six, at least seven, at least eight, at least nine, at least ten, at least eleven, or all twelve of the pan-F-TBRS genes relative to a reference expression level of the at least two, at least three, at least four, at least five, at least six, at least seven, at least eight, at least nine, at least ten, at least eleven, or all twelve pan-F-TBRS genes in the biological sample obtained from the patient.

In examples involving the pan-F-TBRS signature, an expression level below a reference expression level, or a reduced (decreased) expression or number, may refer to an overall reduction of about any of 10%, 20%, 30%, 40%, 50%, 60%, 70%, 80%, 90%, 95%, 96%, 97%, 98%, 99/6 or greater, in the level of biomarker (e.g., protein, nucleic acid (e.g., gene or mRNA), or cell), detected by standard art known methods such as those described herein, as compared to a reference expression level, reference sample, reference cell, reference tissue, control sample, control cell, or control tissue. In certain embodiments, reduced expression or number refers to the decrease in expression level/amount of a biomarker (e.g., one or more of ACTA2, ACTG2, TAGLN, TNS1, CNN1, TPM1, CTGF, PXDC1, ADAM12, FSTL3, TGFBI, and/or ADAM19) in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference expression level, reference sample, reference cell, reference tissue, control sample, control cell, or control tissue. In some embodiments, reduced (decreased) expression or number refers to an overall decrease in expression level/amount of a biomarker (e.g., ACTA2, ACTG2, TAGLN, TNS1, CNN1, TPM1, CTGF, PXDC1, ADAM12, FSTL3, TGFBI, and/or ADAM19) of greater than about 1.1-fold, about 1.2-fold, about 1.3-fold, about 1.4-fold, about 1.5-fold, about 1.6-fold, about 1.7-fold, about 1.8-fold, about 1.9-fold, about 2-fold, about 2.1-fold, about 2.2-fold, about 2.3-fold, about 2.4-fold, about 2.5-fold, about 2.6-fold, about 2.7-fold, about 2.8-fold, about 2.9-fold, about 3-fold, about 3.5-fold, about 4-fold, about 4.5-fold, about 5-fold, about 6-fold, about 7-fold, about 8-fold, about 9-fold, about 10-fold, about 15-fold, about 20-fold, about 30-fold, about 40-fold, about 50-fold, about 100-fold, about 500-fold, about 1,000-fold or greater as compared to a reference expression level, reference sample, reference cell, reference tissue, control sample, control cell, control tissue, or internal control (e.g., housekeeping gene).

In some instances, the expression level of the one or more pan-F-TBRS genes is an mRNA expression level. In other instances, the expression level of the one or more pan-F-TBRS genes is a protein expression level.

In some instances, the biological sample obtained from the patient is a tumor sample.

In some instances, the patient's tumor has a basal-squamous subtype. In some instances, a basal-squamous subtype may be as assessed by The Cancer Genome Atlas (TCGA) classification. TCGA classification may be performed, e.g., as described in Robertson et al. Cell 171(3):540-556, e25, 2017.

In some instances, the patient has an increased expression level of one or more genes selected from CD44, KRT6A, KRT5, KRT14, COL17A1, DSC3, GSDMC, TGM1, and PI3 relative to a reference expression level of the one or more genes.

Any suitable PD-1 axis binding antagonist may be used, including any PD-1 axis binding antagonist known in the art or described in Section IV below. In some instances, the PD-1 axis binding antagonist is selected from a PD-L1 binding antagonist, a PD-1 binding antagonist, and a PD-L2 binding antagonist. In some instances, the PD-1 axis binding antagonist is a PD-L1 binding antagonist. In some instances, the PD-L1 binding antagonist is an anti-PD-L1 antibody. In some instances, the anti-PD-L1 antibody is atezolizumab, durvalumab, avelumab, or MDX-1105. In other instances, the PD-1 axis binding antagonist is a PD-1 binding. In some instances, the PD-1 binding antagonist is an anti-PD-1 antibody. In some instances, the anti-PD-1 antibody is nivolumab, pembrolizumab, MEDI-0680, spartalizumab, cemiplimab, camrelizumab, sintilimab, tislelizumab, toripalimab, or dostarlimab.

In preferred instances, the PD-1 axis binding antagonist is atezolizumab.

For example, in one aspect, provided herein is a method of treating MIUC (e.g., MIBC or muscle-invasive UTUC) in a patient in need thereof, the method comprising: (a) determining whether a patient is ctDNA-positive; and (b) administering an effective amount of a treatment regimen comprising atezolizumab to the patient, wherein the treatment regimen is an adjuvant therapy.

In another aspect, provided herein is atezolizumab, or a pharmaceutical composition comprising atezolizumab, for use in treatment of MIUC (e.g., MIBC or muscle-invasive UTUC) in a patient in need thereof, wherein the treatment comprises administration of an effective amount of a treatment regimen comprising atezolizumab, wherein the treatment regimen is an adjuvant therapy, and wherein the patient is ctDNA-positive.

In another aspect, provided herein is atezolizumab, or a pharmaceutical composition comprising atezolizumab, for use in treatment of MIUC (e.g., MIBC or muscle-invasive UTUC) in a patient in need thereof, the treatment comprising: (a) determining whether a patient is ctDNA-positive; and (b) administering an effective amount of a treatment regimen comprising atezolizumab to the patient, wherein the treatment regimen is an adjuvant therapy.

In any of the preceding aspects, the patient may be determined to be ctDNA-positive post-surgical resection (e.g., cystectomy).

For example, in one aspect, provided herein is a method of adjuvant treatment for MIUC (e.g., MIBC or muscle-invasive UTUC) in a patient in need thereof, wherein the patient has been determined to be ctDNA-positive post-surgical resection, the method comprising administering to the patient an effective amount of a treatment regimen comprising atezolizumab.

In another aspect, provided herein is atezolizumab, or a pharmaceutical composition comprising atezolizumab, for use in adjuvant treatment of MIUC (e.g., MIBC or muscle-invasive UTUC) in a patient in need thereof, wherein the patient has been determined to to be ctDNA-positive post-surgical resection, and wherein the treatment comprises administration of an effective amount of a treatment regimen comprising atezolizumab.

In one aspect, provided herein is a method of adjuvant treatment for MIUC (e.g., MIBC or muscle-invasive UTUC) in a patient in need thereof, wherein the patient has been determined to be ctDNA-positive post-surgical resection, the method comprising administering to the patient an effective amount of a treatment regimen comprising atezolizumab, wherein the treatment regimen comprises administering atezolizumab to the patent at a dose of 1200 mg intravenously (e.g., by infusion) on Day 1 of each 21-day cycle.

In another aspect, provided herein is atezolizumab, or a pharmaceutical composition comprising atezolizumab, for use in adjuvant treatment of MIUC (e.g., MIBC or muscle-invasive UTUC) in a patient in need thereof, wherein the patient has been determined to be ctDNA-positive post-surgical resection, and wherein the treatment comprises administration of an effective amount of a treatment regimen comprising atezolizumab, wherein the treatment regimen comprises administering atezolizumab to the patent at a dose of 1200 mg intravenously (e.g., by infusion) on Day 1 of each 21-day cycle.

In one aspect, provided herein is a method of adjuvant treatment for MIBC in a patient in need thereof, wherein the patient has been determined to be ctDNA-positive post-surgical resection, the method comprising administering to the patient an effective amount of a treatment regimen comprising atezolizumab, wherein the treatment regimen comprises administering atezolizumab to the patent at a dose of 1680 mg intravenously (e.g., by infusion) on Day 1 of each 21-day cycle.

In another aspect, provided herein is atezolizumab, or a pharmaceutical composition comprising atezolizumab, for use in adjuvant treatment of MIBC in a patient in need thereof, wherein the patient has been determined to be ctDNA-positive post-surgical resection, and wherein the treatment comprises administration of an effective amount of a treatment regimen comprising atezolizumab, wherein the treatment regimen comprises administering atezolizumab to the patent at a dose of 1200 mg intravenously (e.g., by infusion) on Day 1 of each 21-day cycle.

In some examples of any of the above aspects, the treatment regimen comprises up to 16 cycles. In other examples, the treatment regimen comprises more than 16 cycles.

In one aspect, provided herein is a method of adjuvant treatment for MIUC (e.g., MIBC or muscle-invasive UTUC) in a patient in need thereof, wherein the patient has been determined to be ctDNA-positive post-surgical resection, the method comprising administering to the patient an effective amount of a treatment regimen comprising atezolizumab, wherein the treatment regimen comprises administering atezolizumab to the patent at a dose of 1680 mg intravenously (e.g., by infusion) on Day 1 of each 28-day cycle.

In another aspect, provided herein is atezolizumab, or a pharmaceutical composition comprising atezolizumab, for use in adjuvant treatment of MIUC (e.g., MIBC or muscle-invasive UTUC) in a patient in need thereof, wherein the patient has been determined to be ctDNA-positive post-surgical resection, and wherein the treatment comprises administration of an effective amount of a treatment regimen comprising atezolizumab, wherein the treatment regimen comprises administering atezolizumab to the patent at a dose of 1680 mg intravenously (e.g., by infusion) on Day 1 of each 28-day cycle.

In one aspect, provided herein is a method of adjuvant treatment for MIBC in a patient in need thereof, wherein the patient has been determined to be ctDNA-positive post-surgical resection, the method comprising administering to the patient an effective amount of a treatment regimen comprising atezolizumab, wherein the treatment regimen comprises administering atezolizumab to the patent at a dose of 1680 mg intravenously (e.g., by infusion) on Day 1 of each 28-day cycle.

In another aspect, provided herein is atezolizumab, or a pharmaceutical composition comprising atezolizumab, for use in adjuvant treatment of MIBC in a patient in need thereof, wherein the patient has been determined to be ctDNA-positive post-surgical resection, and wherein the treatment comprises administration of an effective amount of a treatment regimen comprising atezolizumab, wherein the treatment regimen comprises administering atezolizumab to the patent at a dose of 1680 mg intravenously (e.g., by infusion) on Day 1 of each 28-day cycle.

In some examples of any of the above aspects, the treatment regimen comprises up to 12 cycles. In other examples, the treatment regimen comprises more than 12 cycles.

In any of the preceding aspects, the patient's ctDNA status may be determined in any suitable sample, e.g., a blood sample, a plasma sample, a serum sample, a urine sample, a CSF sample, a nasal swab sample, a saliva sample, a stool sample, or a vaginal fluid sample. In some instances, the sample is a plasma sample.

For example, in one aspect, provided herein is a method of treating MIUC (e.g., MIBC or muscle-invasive UTUC) in a patient in need thereof, the method comprising: (a) determining whether a plasma sample obtained from the patient is ctDNA-positive, wherein a ctDNA-positive plasma sample indicates that the patient is likely to benefit from a treatment regimen comprising atezolizumab; and (b) administering an effective amount of a treatment regimen comprising atezolizumab to the patient based on the plasma sample being ctDNA-positive, wherein the treatment regimen is an adjuvant therapy.

In another aspect, provided herein is atezolizumab, or a pharmaceutical composition comprising atezolizumab, for use in treatment of MIUC (e.g., MIBC or muscle-invasive UTUC) in a patient in need thereof, wherein the treatment comprises administration of an effective amount of a treatment regimen comprising atezolizumab, wherein the treatment regimen is an adjuvant therapy, and wherein the patient has been identified as likely to benefit from the treatment regimen based on a plasma sample obtained from the patient being ctDNA-positive.

In another aspect, provided herein is atezolizumab, or a pharmaceutical composition comprising atezolizumab, for use in treatment of MIUC (e.g., MIBC or muscle-invasive UTUC) in a patient in need thereof, the treatment comprising: (a) determining whether a plasma sample obtained from the patient is ctDNA-positive, wherein a ctDNA-positive plasma sample indicates that the patient is likely to benefit from a treatment regimen comprising atezolizumab; and (b) administering an effective amount of a treatment regimen comprising atezolizumab to the patient based on the plasma sample being ctDNA-positive, wherein the treatment regimen is an adjuvant therapy.

In one aspect, provided herein is a method of adjuvant treatment for MIUC (e.g., MIBC or muscle-invasive UTUC) in a patient in need thereof, wherein the patient has been determined to have a ctDNA-positive plasma sample following cystectomy, the method comprising administering to the patient an effective amount of a treatment regimen comprising atezolizumab.

In another aspect, provided herein is atezolizumab, or a pharmaceutical composition comprising atezolizumab, for use in adjuvant treatment of MIUC (e.g., MIBC or muscle-invasive UTUC) in a patient in need thereof, wherein the patient has been determined to have a ctDNA-positive plasma sample following cystectomy, and wherein the treatment comprises administration of an effective amount of a treatment regimen comprising atezolizumab.

In one aspect, provided herein is a method of adjuvant treatment for MIUC (e.g., MIBC or muscle-invasive UTUC) in a patient in need thereof, wherein the patient has been determined to have a ctDNA-positive plasma sample following cystectomy, the method comprising administering to the patient an effective amount of a treatment regimen comprising atezolizumab, wherein the treatment regimen comprises administering atezolizumab to the patent at a dose of 1200 mg intravenously (e.g., by infusion) on Day 1 of each 21-day cycle.

In another aspect, provided herein is atezolizumab, or a pharmaceutical composition comprising atezolizumab, for use in adjuvant treatment of MIUC (e.g., MIBC or muscle-invasive UTUC) in a patient in need thereof, wherein the patient has been determined to have a ctDNA-positive plasma sample following cystectomy, and wherein the treatment comprises administration of an effective amount of a treatment regimen comprising atezolizumab, wherein the treatment regimen comprises administering atezolizumab to the patent at a dose of 1200 mg intravenously (e.g., by infusion) on Day 1 of each 21-day cycle.

In one aspect, provided herein is a method of adjuvant treatment for MIBC in a patient in need thereof, wherein the patient has been determined to have a ctDNA-positive plasma sample following cystectomy, the method comprising administering to the patient an effective amount of a treatment regimen comprising atezolizumab, wherein the treatment regimen comprises administering atezolizumab to the patent at a dose of 1680 mg intravenously (e.g., by infusion) on Day 1 of each 21-day cycle.

In another aspect, provided herein is atezolizumab, or a pharmaceutical composition comprising atezolizumab, for use in adjuvant treatment of MIBC in a patient in need thereof, wherein the patient has been determined to have a ctDNA-positive plasma sample following cystectomy, and wherein the treatment comprises administration of an effective amount of a treatment regimen comprising atezolizumab, wherein the treatment regimen comprises administering atezolizumab to the patent at a dose of 1200 mg intravenously (e.g., by infusion) on Day 1 of each 21-day cycle.

In some examples of any of the above aspects, the treatment regimen comprises up to 16 cycles. In other examples, the treatment regimen comprises more than 16 cycles.

In one aspect, provided herein is a method of adjuvant treatment for MIUC (e.g., MIBC or muscle-invasive UTUC) in a patient in need thereof, wherein the patient has been determined to have a ctDNA-positive plasma sample following cystectomy, the method comprising administering to the patient an effective amount of a treatment regimen comprising atezolizumab, wherein the treatment regimen comprises administering atezolizumab to the patent at a dose of 1680 mg intravenously (e.g., by infusion) on Day 1 of each 28-day cycle.

In another aspect, provided herein is atezolizumab, or a pharmaceutical composition comprising atezolizumab, for use in adjuvant treatment of MIUC (e.g., MIBC or muscle-invasive UTUC) in a patient in need thereof, wherein the patient has been determined to have a ctDNA-positive plasma sample following cystectomy, and wherein the treatment comprises administration of an effective amount of a treatment regimen comprising atezolizumab, wherein the treatment regimen comprises administering atezolizumab to the patent at a dose of 1680 mg intravenously (e.g., by infusion) on Day 1 of each 28-day cycle.

In one aspect, provided herein is a method of adjuvant treatment for MIBC in a patient in need thereof, wherein the patient has been determined to have a ctDNA-positive plasma sample following cystectomy, the method comprising administering to the patient an effective amount of a treatment regimen comprising atezolizumab, wherein the treatment regimen comprises administering atezolizumab to the patent at a dose of 1680 mg intravenously (e.g., by infusion) on Day 1 of each 28-day cycle.

In another aspect, provided herein is atezolizumab, or a pharmaceutical composition comprising atezolizumab, for use in adjuvant treatment of MIBC in a patient in need thereof, wherein the patient has been determined to have a ctDNA-positive plasma sample following cystectomy, and wherein the treatment comprises administration of an effective amount of a treatment regimen comprising atezolizumab, wherein the treatment regimen comprises administering atezolizumab to the patent at a dose of 1680 mg intravenously (e.g., by infusion) on Day 1 of each 28-day cycle.

In some examples of any of the above aspects, the treatment regimen comprises up to 12 cycles. In other examples, the treatment regimen comprises more than 12 cycles.

In any of the preceding aspects, ctDNA positivity may be determined using a personalized mPCR assay (e.g., a Natera SIGNATERA® assay), in which a plasma sample evaluated to have 2 or more mutations as assessed by the personalized mPCR assay is considered to be ctDNA-positive.

In any of the preceding aspects, ctDNA positivity may be determined using a Food and Drug Administration-approved test.

In some examples, the PD-1 axis binding antagonist is administered as a monotherapy. In other examples, the PD-1 axis binding antagonist is administered in combination with an effective amount of one or more additional therapeutic agents.

In some instances, the method, PD-1 axis binding antagonist for use, pharmaceutical composition for use, or use further comprises administering an additional therapeutic agent to the patient. In some instances, the additional therapeutic agent is selected from the group consisting of an immunotherapy agent, a cytotoxic agent, a growth inhibitory agent, a radiation therapy agent, an anti-angiogenic agent, and combinations thereof.

In any of the preceding examples, each dosing cycle may have any suitable length, e.g., about 7 days, about 14 days, about 21 days, about 28 days, or longer. In some instances, each dosing cycle is about 14 days. In some instances, each dosing cycle is about 21 days. In some instances, each dosing cycle is about 28 days (e.g., 28 days±3 days).

The patient is preferably a human.

As a general proposition, the therapeutically effective amount of a PD-1 axis binding antagonist (e.g., atezolizumab) administered to a human will be in the range of about 0.01 to about 50 mg/kg of patient body weight, whether by one or more administrations.

In some exemplary embodiments, the PD-1 axis binding antagonist is administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, weekly, every two weeks, every three weeks, or every four weeks, for example.

In one instance, a PD-1 axis binding antagonist is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some instances, the PD-1 axis binding antagonist may be administered at a dose of about 1000 mg to about 1400 mg every three weeks (e.g., about 1100 mg to about 1300 mg every three weeks, e.g., about 1150 mg to about 1250 mg every three weeks).

In some instances, a patient is administered a total of 1 to 50 doses of a PD-1 axis binding antagonist, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously. In some instances, a patient is administered a total of 16 doses of a PD-1 axis binding antagonist. In other instances, a patient is administered at total of 12 doses of a PD-1 axis binding antagonist.

In some instances, atezolizumab is administered to the patient intravenously at a dose of about 840 mg every 2 weeks, about 1200 mg every 3 weeks, or about 1680 mg every 4 weeks. In some instances, atezolizumab is administered to the patient intravenously at a dose of 840 mg every 2 weeks. In some instances, atezolizumab is administered to the patient intravenously at a dose of 1200 mg every 3 weeks. In some instances, the atezolizumab is administered on Day 1 of each 21-day (±3 days) cycle for 16 cycles or one year, whichever occurs first. In some instances, atezolizumab is administered to the patient intravenously at a dose of 1680 mg every 4 weeks. In some instances, the atezolizumab is administered on Day 1 of each 28-day (±3 days) cycle for 12 cycles or one year, whichever occurs first.

The PD-1 axis binding antagonist and/or any additional therapeutic agent(s) may be administered in any suitable manner known in the art. For example, the PD-1 axis binding antagonist and/or any additional therapeutic agent(s) may be administered sequentially (on different days) or concurrently (on the same day or during the same treatment cycle). In some instances, the PD-1 axis binding antagonist is administered prior to the additional therapeutic agent. In other instances, the PD-1 axis binding antagonist is administered after the additional therapeutic agent. In some instances, the PD-1 axis binding antagonist and/or any additional therapeutic agent(s) may be administered on the same day. In some instances, the PD-1 axis binding antagonist may be administered prior to an additional therapeutic agent that is administered on the same day. For example, the PD-1 axis binding antagonist may be administered prior to chemotherapy on the same day. In another example, the PD-1 axis binding antagonist may be administered prior to both chemotherapy and another drug (e.g., bevacizumab) on the same day. In other instances, the PD-1 axis binding antagonist may be administered after an additional therapeutic agent that is administered on the same day. In yet other instances, the PD-1 axis binding antagonist is administered at the same time as the additional therapeutic agent. In some instances, the PD-1 axis binding antagonist is in a separate composition as the additional therapeutic agent. In some instances, the PD-1 axis binding antagonist is in the same composition as the additional therapeutic agent. In some instances, the PD-1 axis binding antagonist is administered through a separate intravenous line from any other therapeutic agent administered to the patient on the same day.

The PD-1 axis binding antagonist and any additional therapeutic agent(s) may be administered by the same route of administration or by different routes of administration. In some instances, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some instances, the additional therapeutic agent is administered intravenously, intramuscularly, subcutaneously, topically, orally, transdermally, intraperitoneally, intraorbitally, by implantation, by inhalation, intrathecally, intraventricularly, or intranasally.

In a preferred embodiment, the PD-1 axis binding antagonist is administered intravenously. In one example, atezolizumab may be administered intravenously over 60 minutes; if the first infusion is tolerated, all subsequent infusions may be delivered over 30 minutes. In some examples, the PD-1 axis binding antagonist is not administered as an intravenous push or bolus.

Also provided herein are methods for treating urothelial carcinoma cancer in a patient comprising administering to the patient a treatment regimen comprising an effective amount of a PD-1 axis binding antagonist (e.g., atezolizumab) in combination with another anti-cancer agent or cancer therapy. For example, a PD-1 axis binding antagonist may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof. For example, the PD-1 axis binding antagonist may be administered in combination with bevacizumab, paclitaxel, paclitaxel protein-bound (e.g., nab-paclitaxel), carboplatin, cisplatin, pemetrexed, gemcitabine, etoposide, cobimetinib, vemurafenib, or a combination thereof. The PD-1 axis binding antagonist may be an anti-PD-L1 antibody (e.g., atezolizumab) or an anti-PD-1 antibody.

For example, when administering with chemotherapy with or without bevacizumab, atezolizumab may be administered at a dose of 1200 mg every 3 weeks prior to chemotherapy and bevacizumab. In another example, following completion of 4-6 cycles of chemotherapy, and if bevacizumab is discontinued, atezolizumab may be administered at a dose of 840 mg every 2 weeks, 1200 mg every 3 weeks, or 1680 mg every four weeks. In another example, atezolizumab may be administered at a dose of 840 mg, followed by 100 mg/m² of paclitaxel protein-bound (e.g., nab-paclitaxel); for each 28 day cycle, atezolizumab is administered on days 1 and 15, and paclitaxel protein-bound is administered on days 1, 8, and 15. In another example, when administering with carboplatin and etoposide, atezolizumab can be administered at a dose of 1200 mg every 3 weeks prior to chemotherapy. In yet another example, following completion of 4 cycles of carboplatin and etoposide, atezolizumab may be administered at a dose of 840 mg every 2 weeks, 1200 mg every 3 weeks, or 1680 mg every 4 weeks. In another example, following completion of a 28 day cycle of cobimenitib and vemurafenib, atezolizumab may be administered at a dose of 840 mg every 2 weeks with cobimetinib at a dose of 60 mg orally once daily (21 days on, 7 days off) and vemurafenib at a dose of 720 mg orally twice daily.

In some instances, the treatment may further comprise an additional therapy. Any suitable additional therapy known in the art or described herein may be used. The additional therapy may be radiation therapy, surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

### III. Assessment of PD-L1 Expression

The expression of PD-L1 may be assessed in a patient treated according to any of the methods and compositions for use described herein. The methods and compositions for use may include determining the expression level of PD-L1 in a biological sample (e.g., a tumor sample) obtained from the patient. In other examples, the expression level of PD-L1 in a biological sample (e.g., a tumor sample) obtained from the patient has been determined prior to initiation of treatment or after initiation of treatment. PD-L1 expression may be determined using any suitable approach. For example, PD-L1 expression may be determined as described in U.S. patent application Ser. Nos. 15/787,988 and 15/790,680. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the patient, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the patient has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the patient has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some examples, tumor samples may be scored for PD-L1 positivity in tumor-infiltrating immune cells and/or in tumor cells according to the criteria for diagnostic assessment shown in Table A and/or Table B, respectively.

TABLE A
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by tumor cells, | |
| associated intratumoral stroma, and contiguous | |
| peri-tumoral desmoplastic stroma | |
| Presence of discernible PD-L1 staining of any | IC1 |
| intensity in tumor-infiltrating immune cells | |
| covering ≥1% to <5% of tumor area occupied by | |
| tumor cells, associated intratumoral stroma, | |
| and contiguous peri-tumoral desmoplastic stroma | |
| Presence of discernible PD-L1 staining of any | IC2 |
| intensity in tumor-infiltrating immune cells | |
| covering ≥5% to <10% of tumor area occupied | |
| by tumor cells, associated intratumoral stroma, | |
| and contiguous peri-tumoral desmoplastic stroma | |
| Presence of discernible PD-L1 staining of any | IC3 |
| intensity in tumor-infiltrating immune cells | |
| covering ≥10% of tumor area occupied by tumor | |
| cells, associated intratumoral stroma, and | |
| contiguous peri-tumoral desmoplastic stroma | |

TABLE B
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

### IV. PD-1 Axis Binding Antagonists

PD-1 axis binding antagonists may include PD-L1 binding antagonists, PD-1 binding antagonists, and PD-L2 binding antagonists. Any suitable PD-1 axis binding antagonist may be used.

A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)2 fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:
(a) a heavy chain variable region (VH) comprising the amino acid sequence: EVQLVESGGGLVQPGGSLRLSCAASGFTFSDSWIHWVRQAPGKGLEWVAWISPYGGSTYYADSVKGRF TISADTSKNTAYLQMNSLRAEDTAVYYCARRHWPGGFDYWGQGTLVTVSS (SEQ ID NO: 9), and
(b) the light chain variable region (VL) comprising the amino acid sequence:

(SEQ ID NO: 10)
DIQMTQSPSSLSASVGDRVTITCRASQDVSTAVAWYQQKP
GKAPKLLIYSASFLYSGVPSRFSGSGSGTDFTLTISSLQP
EDFATYYCQQYLYHPATFGQGTKVEIKR

In some instances, the anti-PD-L1 antibody comprises (a) a VH comprising an amino acid sequence comprising having at least 95% sequence identity (e.g., at least 95%, 96%, 97%, 98%, or 99% sequence identity) to, or the sequence of SEQ ID NO: 9; (b) a VL comprising an amino acid sequence comprising having at least 95% sequence identity (e.g., at least 95%, 96%, 97%, 98%, or 99% sequence identity) to, or the sequence of SEQ ID NO: 10; or (c) a VH as in (a) and a VL as in (b).

In one embodiment, the anti-PD-L1 antibody comprises atezolizumab, which comprises:

(a) the heavy chain amino acid sequence:
(SEQ ID NO: 1)
EVQLVESGGGLVQPGGSLRLSCAASGFTFSDSWIHWVRQA
PGKGLEWVAWISPYGGSTYYADSVKGRFTISADTSKNTAY
LQMNSLRAEDTAVYYCARRHWPGGFDYWGQGTLVTVSSAS
TKGPSVFPLAPSSKSTSGGTAALGCLVKDYFPEPVTVSWN
SGALTSGVHTFPAVLQSSGLYSLSSVVTVPSSSLGTQTYI
CNVNHKPSNTKVDKKVEPKSCDKTHTCPPCPAPELLGGPS
VFLFPPKPKDTLMISRTPEVTCVVVDVSHEDPEVKFNWYV
DGVEVHNAKTKPREEQYASTYRVVSVLTVLHQDWLNGKEY
KCKVSNKALPAPIEKTISKAKGQPREPQVYTLPPSREEMT
KNQVSLTCLVKGFYPSDIAVEWESNGQPENNYKTTPPVLD
SDGSFFLYSKLTVDKSRWQQGNVFSCSVMHEALHNHYTQK
SLSLSPG,
and
(b) the light chain amino acid sequence:
(SEQ ID NO: 2)
DIQMTQSPSSLSASVGDRVTITCRASQDVSTAVAWYQQKP
GKAPKLLIYSASFLYSGVPSRFSGSGSGTDFTLTISSLQP
EDFATYYCQQYLYHPATFGQGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC

In some instances, the anti-PD-L1 antibody is avelumab (Chemical Abstract Service (CAS) Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (Medimmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DClg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

V. Detection and Assessment of ctDNA

Provided herein are methods for treating urothelial carcinoma in a patient comprising administering to the patient a treatment regimen comprising a PD-1 axis binding antagonist (e.g., atezolizumab) that involve determining the presence and/or level of ctDNA in a biological sample obtained from the patient. Also provided are related compositions (e.g., pharmaceutical compositions) for use, kits, and articles of manufacture. Any of the methods, compositions for use, kits, or articles of manufacture described herein may involve any suitable approach for detection of ctDNA. In some examples, ctDNA may be detected using a targeted approach (e.g., a PCR-based approach, cancer personalized profiling by deep sequencing (CAPP-Seq) or integrated digital error suppression (iDES) CAPP-Seq, TAM-Seq, Safe-Seq, or duplex sequencing). In other examples, ctDNA may be detected using an untargeted approach (e.g., digital karyotyping, personalized analysis of rearranged ends (PARE), or by detection of DNA methylation and/or hydroxymethylation in ctDNA).

Any suitable biological sample may be used for detection of ctDNA. In some examples, ctDNA may be assessed in blood, serum, or plasma. In a particular example, any of the approaches disclosed herein may involve detection of ctDNA in plasma. In other examples, ctDNA may be assessed in a non-blood sample, e.g., cerebrospinal fluid, saliva, sputum, pleural effusions, urine, stool, or seminal fluid.

ctDNA may be extracted from a biological sample using any suitable approach. For instance, blood may be collected into an EDTA tube and/or a cell stabilization tube (e.g., a Steck tube). The blood may be processed within a suitable amount of time from collection from the patient (e.g., about 2 hours for an EDTA tube or within about 4 days for a cell stabilization tube (e.g., a Steck tube).

As one non-limiting example, ctDNA may be extracted as described in Reinert et al. JAMA Oncol. 5(8):1124-1131, 2019. Briefly, blood samples may be processed within 2 hours of collection into an EDTA tube by double centrifugation of blood at room temperature, first for 10 min at 3000 g, followed by centrifugation of plasma for 10 min at 30000 g. Plasma may be aliquoted into 5 mL cryotubes and stored at −80° C. cfDNA may be extracted using a QIAamp® Circulating Nucleic Acid kit (Qiagen) and eluted into DNA Suspension Buffer (Sigma). cfDNA samples can be quantified, e.g., using a QUANT-iT™ High Sensitivity dsDNA Assay Kit (Invitrogen) or using a fluorometer (e.g., a QUBIT™ fluorometer). Other approaches for extracting ctDNA are known in the art.

In some examples, ctDNA may be detected using a PCR-based approach, a hybridization capture-based approach, a methylation-based approach, or a fragmentomics approach.

In some examples, ctDNA may be detected using a PCR-based approach, e.g., digital PCR (dPCR) (e.g., digital droplet PCR (ddPCR) or BEAMing dPCR). For example, the PCR-based approach may involve detection of one or more mutations associated with cancer (e.g., urothelial carcinoma), e.g., by sequencing (e.g., next-generation sequencing) or mass spectrometry. The PCR-based approach may be targeted or non-targeted. The PCR-based approach may involve detection of somatic variants in a panel of cancer related genes, e.g., a panel including 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, or more genes. Exemplary PCR-based approaches include a personalized ctDNA multiplexed polymerase chain reaction (mPCR) approach, TAM-SEQ™, and Safe-Seq.

In particular examples, a personalized ctDNA multiplexed polymerase chain reaction mPCR approach may be used to detect ctDNA. In some instances, the personalized ctDNA mPCR approach includes one or more (e.g., 1, 2, 3, or all 4) of the following steps: (a) (i) sequencing DNA obtained from a tumor sample obtained from the patient to produce tumor sequence reads; and (ii) sequencing DNA obtained from a normal tissue sample obtained from the patient to produce normal sequence reads; (b) identifying one or more patient-specific variants by calling somatic variants identified from the tumor sequence reads and excluding germline variants and/or CHIP variants, wherein the germline variants or CHIP variants are identified from the normal sequence reads or from a publicly available database; (c) designing an mPCR assay for the patient that detects a set of patient-specific variants; and (d) analyzing a biological sample obtained from the patient using the mPCR assay to determine whether ctDNA is present in the biological sample. In some instances, the sequencing is WES or WGS. In some instances, the sequencing is WES. In some instances, patient-specific variants are SNVs or short indels. In some instances, patient-specific variants are SNVs. In some instances, the set of patient-specific variants comprises at least 2 (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) patient-specific variants. In some instances, the set of patient-specific variants comprises at least 1 patient-specific variant. In some instances, the set of patient-specific variants comprises at least 2 patient-specific variants. In some instances, the set of patient-specific variants comprises at least 8 patient-specific variants. In some instances, the set of patient-specific variants comprises 2 to 200 patient-specific variants. In some instances, the set of patient-specific variants comprises 8 to 50 patient-specific variants. In some instances, the set of patient-specific variants comprises 8 to 32 patient-specific variants. In some instances, the set of patient-specific variants comprises 16 patient-specific variants. In some instances, analyzing the biological sample obtained from the patient using the mPCR assay comprises sequencing amplicons produced by the mPCR assay to identify patient-specific variants in the biological sample. In some instances, presence of at least one patient-specific variant in the biological sample identifies the presence of ctDNA in the biological sample. In some instances, the presence of 1, 2, 3, 4, 5, 6, 7, 8, 9, 10, 15, 20, 25, 30, 35, 40, 45, 50, or more patient-specific variants in the biological sample identifies the presence of ctDNA in the biological sample. In particular instances, the presence of 2 patient-specific variants in the biological sample identifies the presence of ctDNA in the biological sample. In some instances, the presence of 0 or 1 patient-specific variants in the biological sample indicates that ctDNA is absent from the biological sample.

In some instances, the personalized ctDNA mPCR approach is a Natera SIGNATERA® ctDNA test or an ArcherDx Personalized Cancer Monitoring (PCM™) test. In some examples, the personalized ctDNA mPCR approach may be as described in one or more of U.S. Pat. Nos. 10,538,814; 10,557,172; 10,590,482; and/or 10,597,708.

In other examples, ctDNA may be detected using a hybridization capture-based approach, e.g., by cancer personalized profiling by deep sequencing (CAPP-Seq) (see, e.g., Newman et al. Nat. Med. 20(5):548-554, 2014) or integrated digital error suppression (iDES) CAPP-Seq (see, e.g., Newman et al. Nat. Biotechnol. 34(5):547-555, 2016).

In other examples, ctDNA may be detected using a methylation or fragmentomics approach (e.g., a Guardant LUNAR assay, a GRAIL assay, a Freenome assay, or cell-free methylated DNA immunoprecipitation and high-throughput sequencing (cfMeDIP-seq) (see, e.g., Nuzzo et al. Nature Med. 26:1041-1043, 2020)). Methylation-based approaches may include, e.g., a whole-genome bisulfite sequencing approach or a targeted methylation assay. In some examples, the methylation approach includes a targeted methylation assay such as a GRAIL assay (see, e.g., Liu et al. Annals Oncol. 31(6):745-759, 2020). In some examples, a methylation-based approach may also provide tissue-of-origin information (see, e.g., Liu et al. supra and Guo et al. Nat. Genet. 49(4):635-642, 2017).

### VI. Assessment of TMB

Provided herein are methods for treating urothelial carcinoma (e.g., MIUC) in a patient comprising administering to the patient a treatment regimen comprising a PD-1 axis binding antagonist (e.g., atezolizumab) that involve determining a tTMB score in a sample obtained from the patient. Also provided are related compositions (e.g., pharmaceutical compositions) for use, kits, and articles of manufacture. Any of the methods, compositions for use, kits, or articles of manufacture described herein may involve any suitable approach for determination of a tTMB score. For example, a tTMB score may be determined using whole-exome sequencing, whole-genome sequencing, or by using a targeted panel (e.g., the FOUNDATIONONE® panel). For example, in some instances, WES may be used to both design a personalized mPCR assay to detect ctDNA and to determine a patient's tTMB score. In some aspects, a tTMB score may be determined as disclosed in International Patent Application Publication No. PCT/US2017/055669, which is incorporated by reference herein in its entirety. In other aspects, a bTMB score may be determined in a blood sample obtained from the patient. Any suitable approach may be used to determine a patient's bTMB score. For example, in some aspects, a bTMB score may be determined as described in International Patent Application Publication No. PCT/US2018/043074, which is incorporated by reference herein in its entirety.

In some aspects, a tumor sample obtained from the patient has been determined to have a tissue tTMB score that is at or above a reference tTMB score. Any suitable reference tTMB score may be used.

In some instances, the reference tTMB score is a tTMB score in a reference population of individuals having urothelial carcinoma, wherein the population of individuals consists of a first subset of individuals who have been treated with a PD-1 axis binding antagonist therapy and a second subset of individuals who (i) have not been treated or (ii) have been treated with a non-PD-L1 axis binding antagonist therapy, which does not comprise a PD-L1 axis binding antagonist. In some instances, the reference tTMB score significantly separates each of the first and second subsets of individuals based on a significant difference in responsiveness to treatment with the PD-L1 axis binding antagonist therapy relative to responsiveness (i) in the absence of treatment or (ii) to treatment with the non-PD-L1 axis binding antagonist therapy. Responsiveness may be in terms of improved ORR, CR rate, pCR rate, PR rate, improved survival (e.g., DFS, DSS, distant metastasis-free survival, PFS and/or OS), improved DOR, improved time to deterioration of function and QoL, and/or ctDNA clearance. Improvement (e.g., in terms of response rate (e.g., ORR, CR, and/or PR), survival (e.g., DFS, DSS, distant metastasis-free survival, PFS, and/or OS), DOR, improved time to deterioration of function and QoL, and/or ctDNA clearance) may be relative to a suitable reference, for example, observation or a reference treatment (e.g., treatment that does not include the PD-1 axis binding antagonist (e.g., treatment with placebo)). In some instances, improvement (e.g., in terms of response rate (e.g., ORR, CR, and/or PR), survival (e.g., DFS, DSS, distant metastasis-free survival, PFS, and/or OS), or DOR) may be relative to observation.

In some instances, the reference tTMB score is a pre-assigned tTMB score. In some instances, the reference tTMB score is between about 5 and about 100 mutations per Mb (mut/Mb), for example, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 mut/Mb. For example, in some instances, the reference tTMB score is between about 8 and about 30 mut/M (e.g., about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mut/Mb). In some instances, the reference tTMB score is between about 10 and about 20 mut/Mb (e.g., about 10, about 11, about 12, about 13, about 14, about 15, about 16, about 17, about 18, about 19, or about 20 mut/Mb). In particular instances, the reference tTMB score may be 10 mut/Mb, 16 mut/Mb, or 20 mut/Mb. In particular instances, the reference tTMB score may be 10 mut/Mb. The reference tTMB score may be an equivalent tTMB value to any of the foregoing pre-assigned tTMB scores.

In some instances, the tumor sample from the patient has a tTMB score of greater than, or equal to, about 5 mut/Mb. For example, in some instances, the tTMB score from the tumor sample is between about 5 and about 100 mut/Mb (e.g., about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 mut/M). In some instance, the tumor sample from the patient has a tTMB score of greater than, or equal to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 mut/Mb. For example, in some instances, the tumor sample from the patient has a tTMB score of greater than, or equal to, about 10 mut/Mb. In some embodiments, the reference tTMB score is 10 mut/Mb. In some instances, the tTMB score from the tumor sample is between about 10 and 100 mut/Mb. In some instances, the tTMB score from the tumor sample is between about 10 and 20 mut/Mb. In some instances, the tumor sample from the patient has a tTMB score of greater than, or equal to, about 16 mut/Mb. In some instances, the tumor sample from the patient has a tTMB score of greater than, or equal to, about 16 mut/Mb, and the reference tTMB score is 16 mut/Mb. In other instances, the tumor sample from the patient has a tTMB score of greater than, or equal to, about 20 mut/Mb. In some instances, the tumor sample from the patient has a tTMB score of greater than, or equal to, about 20 mut/Mb, and the reference tTMB score is about 20 mut/Mb.

In some instances, the tTMB score or the reference tTMB score is represented as the number of somatic mutations counted per a defined number of sequenced bases. For example, in some instances, the defined number of sequenced bases is between about 100 kb to about 10 Mb. In some instances, the defined number of sequenced bases is about 1.1 Mb (e.g., about 1.125 Mb), e.g., as assessed by the FOUNDATIONONE® panel). In some instances, the tTMB score or the reference tTMB score is an equivalent TMB value. In some instances, the equivalent TMB value is determined by WES. In other instances, the equivalent TMB value is determined by WGS.

In some instances, the test simultaneously sequences the coding region of about 300 genes (e.g., a diverse set of at least about 300 to about 400 genes, e.g., about 300, 310, 320, 330, 340, 350, 360, 370, 380, 390, or 400 genes) covering at least about 0.05 Mb to about 10 Mb (e.g., 0.05, 0.06. 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 Mb) to a typical median depth of exon coverage of at least about 500× (e.g., 500×, 550×, 600×, 650×, 700×, 750×, 800×, 850×, 900×, 950×, or 1,000×). In other instances, the test simultaneously sequences the coding regions of about 400 genes, about 425 genes, about 450 genes, about 475 genes, about 500 genes, about 525 genes, about 550 genes, about 575 genes, about 600 genes, about 625 genes, about 650 genes, about 675 genes, about 700 genes, about 725 genes, about 750 genes, about 775 genes, about 800 genes, about 825 genes, about 850 genes, about 875 genes, about 900 genes, about 925 genes, about 950 genes, about 975 genes, about 1000 genes, or greater than 1000 genes. In some instances, the set of genes is the set of genes of the FOUNDATIONONE® panel (see, e.g., Frampton et al. Nat. Biotechnol. 31:1023-31, 2013, which is incorporated herein by reference in its entirety). In some instances, the set of genes is the set of genes of the FOUNDATIONONE® CDx panel. In some embodiments, the test sequences greater than about 10 Mb of the genome of the individual, e.g., greater than about 10 Mb, greater than about 15 Mb, greater than about 20 Mb, greater than about 25 Mb, greater than about 30 Mb, greater than about 35 Mb, greater than about 40 Mb, greater than about 45 Mb, greater than about 50 Mb, greater than about 55 Mb, greater than about 60 Mb, greater than about 65 Mb, greater than about 70 Mb, greater than about 75 Mb, greater than about 80 Mb, greater than about 85 Mb, greater than about 90 Mb, greater than about 95 Mb, greater than about 100 Mb, greater than about 200 Mb, greater than about 300 Mb, greater than about 400 Mb, greater than about 500 Mb, greater than about 600 Mb, greater than about 700 Mb, greater than about 800 Mb, greater than about 900 Mb, greater than about 1 Gb, greater than about 2 Gb, greater than about 3 Gb, or about 3.3 Gb. In some instances, the test simultaneously sequences the coding region of 315 cancer-related genes plus introns from 28 genes often rearranged or altered in cancer to a typical median depth of coverage of greater than 500×. In some instances, each covered sequencing read represents a unique DNA fragment to enable the highly sensitive and specific detection of genomic alterations that occur at low frequencies due to tumor heterogeneity, low tumor purity, and small tissue samples. In other instances, the presence and/or level of somatic mutations is determined by WES. In some instances, the presence and/or level of somatic mutation is determined by WGS.

The patient's tTMB score may be determined based on the number of somatic alterations in a tumor sample obtained from the patient. In certain instances, the somatic alteration is a silent mutation (e.g., a synonymous alteration). In other instances, the somatic alteration is a non-synonymous SNV. In other instances, the somatic alteration is a passenger mutation (e.g., an alteration that has no detectable effect on the fitness of a clone). In certain instances, the somatic alteration is a variant of unknown significance (VUS), for example, an alteration, the pathogenicity of which can neither be confirmed nor ruled out. In certain instances, the somatic alteration has not been identified as being associated with a cancer phenotype.

In certain instances, the somatic alteration is not associated with, or is not known to be associated with, an effect on cell division, growth, or survival. In other instances, the somatic alteration is associated with an effect on cell division, growth, or survival.

In certain instances, the number of somatic alterations excludes a functional alteration in a sub-genomic interval.

In some instances, the functional alteration is an alteration that, compared with a reference sequence (e.g., a wild-type or unmutated sequence) has an effect on cell division, growth, or survival (e.g., promotes cell division, growth, or survival). In certain instances, the functional alteration is identified as such by inclusion in a database of functional alterations, e.g., the COSMIC database (see Forbes et al. Nucl. Acids Res. 43 (D1): D805-D811, 2015, which is herein incorporated by reference in its entirety). In other instances, the functional alteration is an alteration with known functional status (e.g., occurring as a known somatic alteration in the COSMIC database). In certain instances, the functional alteration is an alteration with a likely functional status (e.g., a truncation in a tumor suppressor gene). In certain instances, the functional alteration is a driver mutation (e.g., an alteration that gives a selective advantage to a clone in its microenvironment, e.g., by increasing cell survival or reproduction). In other instances, the functional alteration is an alteration capable of causing clonal expansions. In certain instances, the functional alteration is an alteration capable of causing one, two, three, four, five, or all six of the following: (a) self-sufficiency in a growth signal; (b) decreased, e.g., insensitivity, to an antigrowth signal; (c) decreased apoptosis; (d) increased replicative potential; (e) sustained angiogenesis; or (f) tissue invasion or metastasis.

In certain instances, the functional alteration is not a passenger mutation (e.g., is not an alteration that has no detectable effect on the fitness of a clone of cells). In certain instances, the functional alteration is not a variant of unknown significance (VUS) (e.g., is not an alteration, the pathogenicity of which can neither be confirmed nor ruled out).

In certain instances, a plurality (e.g., about 10%, 20%, 30%, 40%, 50%, 60%, 70%, 80%, 90%, or more) of functional alterations in a pre-selected tumor gene in the pre-determined set of genes are excluded. In certain instances, all functional alterations in a pre-selected gene (e.g., tumor gene) in the pre-determined set of genes are excluded. In certain instances, a plurality of functional alterations in a plurality of pre-selected genes (e.g., tumor genes) in the pre-determined set of genes are excluded. In certain instances, all functional alterations in all genes (e.g., tumor genes) in the pre-determined set of genes are excluded.

In certain instances, the number of somatic alterations excludes a germline mutation in a sub-genomic interval.

In certain instances, the germline alteration is an SNP, a base substitution, an insertion, a deletion, an indel, or a silent mutation (e.g., synonymous mutation).

In certain instances, the germline alteration is excluded by use of a method that does not use a comparison with a matched normal sequence. In other instances, the germline alteration is excluded by a method comprising the use of an algorithm. In certain instances, the germline alteration is identified as such by inclusion in a database of germline alterations, for example, the dbSNP database (see Sherry et al. Nucleic Acids Res. 29(1): 308-311, 2001, which is herein incorporated by reference in its entirety). In other instances, the germline alteration is identified as such by inclusion in two or more counts of the ExAC database (see Exome Aggregation Consortium et al. bioRxiv preprint, Oct. 30, 2015, which is herein incorporated by reference in its entirety). In some instances, the germline alteration is identified as such by inclusion in the 1000 Genome Project database (McVean et al. Nature 491, 56-65, 2012, which is herein incorporated by reference in its entirety). In some instances, the germline alteration is identified as such by inclusion in the ESP database (Exome Variant Server, NHLBI GO Exome Sequencing Project (ESP), Seattle, WA).

### VII. Pharmaceutical Compositions and Formulations

Also provided herein are pharmaceutical compositions and formulations comprising a PD-1 axis binding antagonist (e.g., atezolizumab) and, optionally, a pharmaceutically acceptable carrier.

Pharmaceutical compositions and formulations as described herein can be prepared by mixing the active ingredients (e.g., a PD-1 axis binding antagonist) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary atezolizumab formulation comprises glacial acetic acid, L-histidine, polysorbate 20, and sucrose, with a pH of 5.8. For example, atezolizumab may be provided in a 20 mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14 mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

### VIII. Articles of Manufacture or Kits

In another aspect, provided herein is an article of manufacture or a kit comprising a PD-1 axis binding antagonist (e.g., atezolizumab). In some instances, the article of manufacture or kit further comprises package insert comprising instructions for using the PD-1 axis binding antagonist to treat or delay progression of urothelial carcinoma in a patient. In some instances, the article of manufacture or kit further comprises package insert comprising instructions for using the PD-1 axis binding antagonist in combination with one or more additional therapeutic agents to treat or delay progression of urothelial carcinoma cancer in a patient. Any of the PD-1 axis binding antagonists and/or any additional therapeutic agents described herein may be included in the article of manufacture or kits.

In some instances, the PD-1 axis binding antagonist and any additional therapeutic agent(s) are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agent include, for example, bottles, vials, bags and syringes.

Any of the articles of manufacture or kits may include instructions to administer a PD-1 axis binding antagonist and/or any additional therapeutic agents to a patient in accordance with any of the methods described herein, e.g., any of the methods set forth in Section II above.

In another aspect, provided herein is an article of manufacture comprising a PD-1 axis binding antagonist and instructions to administer the PD-1 axis binding antagonist for treatment of urothelial carcinoma in a patient in need thereof, wherein the treatment comprises administration of an effective amount of a treatment regimen comprising a PD-1 axis binding antagonist, wherein the treatment regimen is an adjuvant therapy, and wherein the patient has been identified as likely to benefit from the treatment regimen based on the presence of ctDNA in a biological sample obtained from the patient.

In another aspect, provided herein is an article of manufacture comprising a PD-1 axis binding antagonist and instructions to administer the PD-1 axis binding antagonist for treatment of urothelial carcinoma in a patient in need thereof, the treatment comprising: (a) determining whether ctDNA is present in a biological sample obtained from the patient, wherein the presence of ctDNA in the biological sample indicates that the patient is likely to benefit from a treatment regimen comprising a PD-1 axis binding antagonist; and (b) administering an effective amount of a treatment regimen comprising a PD-1 axis binding antagonist to the patient based on the presence of ctDNA in the biological sample, wherein the treatment regimen is an adjuvant therapy.

In another aspect, provided herein is an article of manufacture comprising a PD-1 axis binding antagonist and instructions to administer the PD-1 axis binding antagonist for treatment of a patient having a urothelial carcinoma who has been administered at least a first dose of a treatment regimen comprising a PD-1 axis binding antagonist, wherein the treatment regimen is a neoadjuvant therapy or an adjuvant therapy, and wherein ctDNA was present in a biological sample obtained from the patient prior to or concurrently with the first dose of the treatment regimen. In some embodiments, the treatment regimen is a neoadjuvant therapy. In other embodiments, the treatment regimen is an adjuvant therapy.

### Examples

### Example 1: Clinical Outcomes in ctDNA-Positive Urothelial Carcinoma Patients Treated with Adjuvant Immunotherapy

Historically, it has been difficult to determine after surgery which patients harbor residual disease and which are cured, despite advances in tumor staging, radiologic imaging, and tissue-based prognostic biomarkers. As a consequence of this, many patients cured by surgery are unnecessarily exposed to adjuvant therapy toxicities, and other patients with residual disease may not receive additional treatment until disease progression is detectable by imaging (perhaps missing an opportunity to receive timely adjuvant therapy with curative intent). Detection of ctDNA shortly after surgical resection may overcome these limitations by enabling early identification of patients harboring minimal residual disease (MRD), at highest risk of radiological relapse. Whether MRD status as assessed by ctDNA can identify which patients may benefit from adjuvant therapy, and which patients can be spared additional treatment, has yet to be investigated in a randomized setting.

This Example describes results from IMvigor010 (NCT02450331), which was a global, Phase III, open-label, randomized trial of atezolizumab as adjuvant treatment of patients with high risk muscle invasive urothelial carcinoma (MIUC) of either the bladder or upper tract. IMvigor010 did not show significant disease-free survival (DFS) benefit in unselected patients, nor overall survival (OS) benefit. Therefore, this was an ideal setting to investigate the question of whether MRD(+) patients by ctDNA, who have a high likelihood of recurrence, can derive clinical benefit from adjuvant treatment with immune checkpoint inhibition (e.g., with a PD-1 axis binding antagonist such as atezolizumab).

A. Objectives and Endpoints

i. Primary Efficacy Objective

The primary efficacy objective for this study was to evaluate the efficacy of adjuvant treatment with atezolizumab compared with observation in MIUC on the basis of DFS, which was defined by local (pelvic) or urinary tract recurrence, distant UC metastasis or death from any cause.

ii. Secondary Efficacy Objective

A secondary efficacy objective for this study was to evaluate the efficacy of adjuvant treatment with atezolizumab compared with observation in MIUC on the basis of OS, which was defined by the time from randomization to death from any cause.

iii. Exploratory Efficacy Objective

A prospective exploratory objective for this study was to evaluate the utility of ctDNA to identify patients who may benefit from atezolizumab treatment. ctDNA was measured at the start of therapy (C1D1) and at week 9 (C3D1).

B. Study Design

IMvigor010 was a global, Phase III, open-label, randomized, controlled study designed to evaluate the efficacy and safety of adjuvant treatment with atezolizumab compared to observation in 809 people with MIUC, who were at high risk for recurrence following resection. The primary endpoint was DFS as assessed by investigator, which was defined as the time from randomization to invasive urothelial cancer recurrence or death.

Patients were randomized 1:1 to either atezolizumab or observation arms. Treatment with atezolizumab (1200 mg every 3 weeks) was administered (or patients underwent observation) for 1 year or until UC recurrence or unacceptable toxicity. Imaging assessments for disease recurrence were performed at baseline and every 12 weeks for 3 years, every 24 weeks for years 4-5, and at year 6. Disease recurrence assessments for patients in the observation arm followed the same schedule as those in the atezolizumab arm. This study enrolled 809 patients (406 atezolizumab and 403 observation). There were 581 patients included in the ctDNA C1D1 Biomarker Evaluable Population (BEP, 72% of the intent-to-treat (ITT) population).

Crossover was not permitted between the atezolizumab and observation arms.

Tumor tissue was collected from surgical resection samples, where formalin fixed paraffin-embedded (FFPE) tissue blocks were preferred (n=138), followed by archival unstained FFPE tissue slides (n=443). Central evaluation for PD-L1 expression was conducted using the VENTANA SP142 IHC assay. Tumors were classified as expressing PD-L1 (IC2/3 status) when PD-L1-expressing tumor-infiltrating immune cells covered a 5% of the tumor area.

C. Materials and Methods

i. Patients

A total of 809 patients were enrolled in the IMvigor010 study (406 atezolizumab and 403 observation). There were 581 patients included in the ctDNA C1D1 BEP (72% of the ITT population).

ii. Inclusion Criteria

Inclusion criteria required patients to be high-risk at pathologic staging (pT3-T4a or N+ for patients not treated with neoadjuvant chemotherapy, or pT2-T4a or N+ for patients treated with neoadjuvant chemotherapy). Patients were required to have undergone surgical resection (cystectomy or nephroureterectomy) with lymph node dissection, with no evidence of residual disease or metastasis as confirmed by negative postoperative radiologic imaging.

iii. Study Treatment

Treatment with atezolizumab (1200 mg every 3 weeks) was administered (or patients underwent observation) for 1 year or until UC recurrence or unacceptable toxicity. Imaging assessments for disease recurrence were performed at baseline and every 12 weeks for 3 years, every 24 weeks for years 4-5, and at year 6. Disease recurrence assessments for patients in the observation arm followed the same schedule as those in the atezolizumab arm. Crossover was not permitted between the atezolizumab and observation arms.

iv. Interim Analysis

The median follow-up was 21.9 months (calculated using the reverse Kaplan-Meier approach), with a range of 16-45 months. At the data cutoff, OS follow-up was immature and ongoing in the ITT population. The median OS was not reached in the interim analysis; 118 patients (29.1%) in the atezolizumab arm and 124 patients in the observation arm (30.8%) died. 33.3% and 29.6% of patients who relapsed received subsequent cancer therapy in the atezolizumab and observation arm respectively. This included chemotherapy in 25.6 and 24.3% respectively and immune therapy in 8.6% and 20.3% respectively, and represents expected treatment patterns for front line advanced disease.

v. Blood Collection and Processing

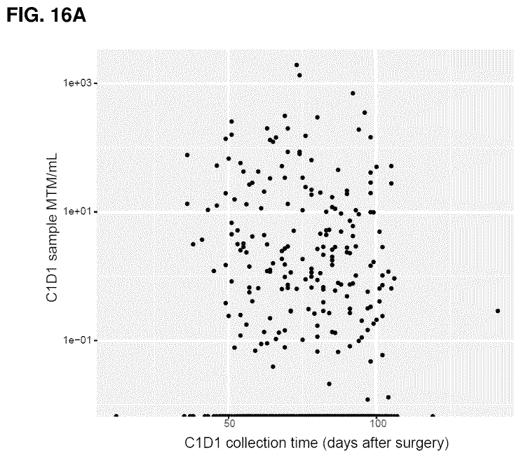

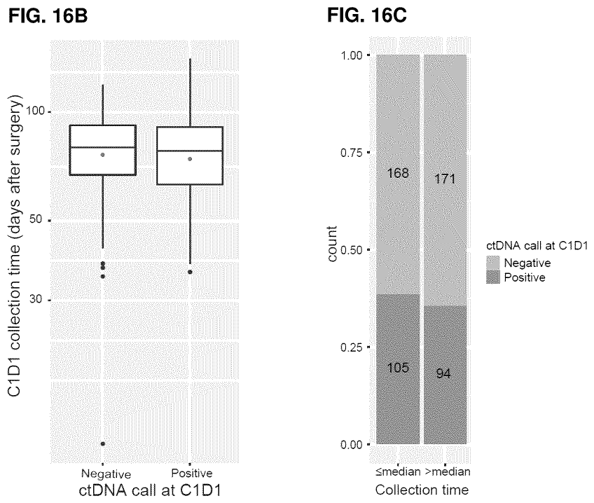

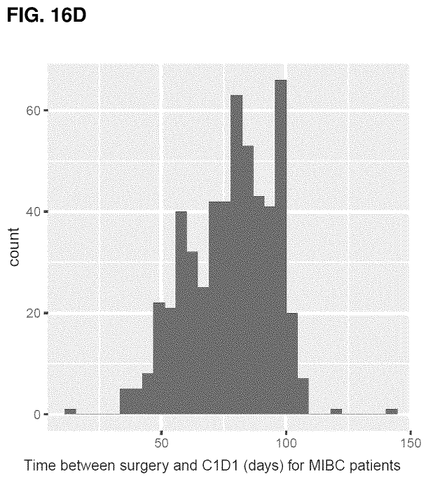

The Cycle 1 Day 1 (C1D1) plasma timepoint was collected at a median of 79 days post-surgical resection (IQR 6592 days for MIBC patients), which did not correlate with ctDNA levels (FIGS. 16A-16D). Collection time analyses were conducted for patients with MIBC only, because patients with upper-tract UC often received two surgeries. Peripheral blood mononuclear cells (PBMC) were collected in three 8.5 mL ACD tubes at the beginning of C1D1, and peripheral blood plasma was collected in two 6 mL EDTA tubes at the beginning of C1D1 and C3D1. Plasma was separated from the cell pellet within 30 minutes of collection and aliquoted for storage at −80° C. Note that the Natera assay used in this study is validated for frozen plasma utilizing spun-down K2-EDTA collected blood samples within 2 hours of collection, however the clinical version of the assay utilizes Streck collection tubes that stabilize cfDNA and allow for ambient shipment within 7 days. A total of 1076 plasma samples (581 from C1D1, 495 from C3D1) from 591 patients were used in this analysis with medians of 3.7 mL of plasma used (IQR 3.2-4.2 mL) and 21.5 ng cfDNA extracted (IQR 13.2-34.2 ng) per patient. To identify ctDNA in a patient's plasma, cfDNA extraction and library prep steps were performed (see, e.g., Reinert et al. JAMA Oncol. 5(8): 1124-31 (2019)).

vi. Tumor Tissue Processing

Tumor tissue was collected from surgical resection samples, where formalin fixed paraffin-embedded (FFPE) tissue blocks were preferred (n=138), followed by archival unstained FFPE tissue slides (n=443). Genomic DNA was extracted using the QIAamp® DNA FFPE Tissue Kit. Central evaluation for PD-L1 expression was conducted using the VENTANA SP142 IHC assay. Tumors were classified as expressing PD-L1 (IC2/3 status) when PD-L1-expressing tumor-infiltrating immune cells covered ≥5% of the tumor area.

vii. Whole Exome Sequencing of Tumor Tissue and Matched Normal DNA

A median of 500 ng of genomic DNA (gDNA) was used for the whole exome sequencing workflow for both tumor and normal sources. An Illumina-adapter based library preparation was performed on this gDNA. Targeted exome capture was then performed using a custom capture probe set that targets ˜19,500 genes. These targeted libraries were sequenced on the NovaSeq™ platform at 2×100 bp to achieve the deduplicated on-target average coverage of 180× for tumor tissue and 50× for the associated matched normal sample. FastQ files were prepared using bcl2fastq2 and quality checked using FastQC. Reads were mapped to the human reference genome hg19 using Burrows-Wheeler Alignment tool (v.0.7.12) and quality checked using Picard and MultiQC.

viii. Somatic Variant Calling and SIGNATERA® ctDNA Assay Design

Using the input of tumor tissue and matched normal sequencing data, somatic variant calling was performed using a consensus variant calling method developed by Natera. Variants previously reported to be germline in public datasets (1000 Genome project, ExAC, ESP, dbSNP) were filtered out, and other collections were also filtered out. The WES data from paired tumor and matched normal were first analyzed for quality metrics and sample concordance and then processed through a bioinformatics pipeline that allows identification of putative clonal somatic single nucleotide variants. Matched normal sequencing was done to computationally remove putative germline and clonal hematopoiesis of indeterminate potential mutations. Out of the candidate pool of putative clonal variants specific to the tumor DNA of each patient, a prioritized list of variants was used to design PCR amplicons based on optimized design parameters, ensuring uniqueness in the human genome, amplicon efficiency and primer interaction. Tumor mutational burden (TMB) was calculated as the total number of somatic mutations per megabase of captured exome, and TMB positive patients were those with ≥10 mutations/Mb (the mean of the ctDNA BEP).

Following plasma cfDNA extraction and library prep, multiplexed targeted PCR was conducted on an aliquot of cfDNA library, followed by amplicon-based sequencing and to an average next-generation sequencing depth per amplicon of >100,000× on an Illumina platform. On observing at least 2 or more mutations in the patient's plasma, the patient was deemed ctDNA-positive (Coombes et al. Clin Cancer Res. 25(14): 4255-4263 (2019)). ctDNA(+) samples additionally have reported the sample Mean Tumor Molecules per mL of plasma (sample MTM/mL), which is the average of tumor molecules across all variants that meet QC requirements per mL of plasma. Analytical studies of the Natera SIGNATERA® assay, as previously published, have demonstrated a >95% sensitivity at 0.01% variant allele frequency with high specificity (Coombes et al. Clin Cancer Res. 25(14): 4255-4263 (2019)). The turnaround time for the SIGNATERA® assay is (i) 2-3 weeks for the first plasma sample, including tissue WES, assay design, and plasma ctDNA analysis/reporting and (ii) one week for all subsequent plasma processing and ctDNA analysis/reporting.

ix. RNA Processing

Formalin-fixed paraffin-embedded (FFPE) tissue was macro-dissected for tumor area using hematoxylin and eosin (H&E) as a guide. RNA was extracted using the High Pure FFPET RNA Isolation Kit (Roche) and assessed by Qubit and Agilent Bioanalyzer for quantity and quality. First strand cDNA synthesis was primed from total RNA using random primers, followed by the generation of second strand cDNA with dUTP in place of dTTP in the master mix to facilitate preservation of strand information. Libraries were enriched for the mRNA fraction by positive selection using a cocktail of biotinylated oligos corresponding to coding regions of the genome. Libraries were sequenced using the Illumina sequencing method.

x. RNA-seq Data Generation and Processing

Whole-transcriptome profiles were generated using TruSeq® RNA Access technology (Illumina). RNA-seq reads were first aligned to ribosomal RNA sequences to remove ribosomal reads. The remaining reads were aligned to the human reference genome (NCBI Build 38) using GSNAP (Wu and Nacu. Bioinformatics. 26(7): 873-881 (2010); Wu et al. Methods Mol Biol. 1418: 283-334 (2016)) version 2013-10-10, allowing a maximum of two mismatches per 75 base sequence (parameters: ‘-M 2 -n 10 -B 2 -i 1 -N 1 -w 200000 -E 1-pairmax-rna=200000 -clip-overlap). To quantify gene expression levels, the number of reads mapped to the exons of each RefSeq gene was calculated using the functionality provided by the R/Bioconductor package GenomicAlignments. Raw counts were adjusted for gene length using transcript-per-million (TPM) normalization, and subsequently log 2-transformed. Raw and processed data were available under the data sharing agreement for N=728 patients with RNA-seq data available. All analyses in this study used N=573 patients with both RNA-seq and ctDNA data available.

xi. Unsupervised mRNA Expression Clustering

TCGA subtypes were assigned according to the methodology described previously (Robertson et al. Cell. 171(3): 540-556.e25 (2017)). Briefly, RNA expression data for samples were normalized using trimmed mean of M-values normalization and transformed with voom, resulting in log 2-counts per million with associated precision weights. The top 25% most-varying genes, ranked by standard deviation across all samples considered were selected. The log₂ normalized expression of 4660 genes were median centered before performing consensus clustering, categorizing the samples into five clusters. The expression clustering analysis was done with a consensus hierarchical clustering approach using the distance matrix of 1−C, the element Cᵢⱼ representing the Spearman correlation between the sample i and j across 4660 genes in R. A consensus matrix M_{K}, K=5 being the number of clusters, was computed by iterating a standard hierarchical clustering (K×500) times with the average linkage option and 80% resampling in sample space. The clustering recapitulated the five distinct clusters as reported in Robertson et al. Cell 171(3): 540-556.e25 (2017), as indicated by the signatures shown on the heatmap.

xii. Gene Set Enrichment Analysis (GSEA)

GSEA ranks all of the genes in the dataset based on differential expression. GSEA was performed followed by applying the CAMERA enrichment method (Wu and Smyth. Nucleic Acids Res. 40(17): e133 (2012)) to perform a competitive test to assess whether the genes in a given set are highly ranked in terms of differential expression relative to genes that are not in the set. The Hallmark gene set collection from the Molecular Signature Database (Subramanian et al. Proc Nat Acad Sci USA 102(43): 15545-15550 (2005)) was used to identify the pathways enriched. Pathways with adjusted P values<0.05 were included.

xiii. Statistical Analysis

The ctDNA statistical analysis plan (ctDNA SAP) was planned and finalized before unblinding of clinical data for primary trial analysis. The Primary Objectives for the ctDNA study were to provide evidence that 1) in the ctDNA positive patients at C1D1, atezolizumab provided improvedDFS compared to observation arm, 2) the presence of ctDNA in plasma at C1D1 is associated with decreased DFS, 3) the presence of ctDNA in plasma at C3D1 is associated with decreased DFS, and 4a) the clearance of ctDNA in plasma by C3D1 is associated with increased DFS and 4b) clearance occurs at a higher rate in atezolizumab arm compared to observation arm. Clearance is defined in this study as going from ctDNA(+) at C1 to ctDNA(−) at C3, and is assessed only in patients who are ctDNA(+) at C1. Primary analysis used a univariable approach with categorical ctDNA (ctDNA+/−). Secondary objectives included ctDNA as a continuous variable (sample mean tumor molecules per mL of plasma), and a multivariable approach adjusting for known risk factors. Secondary endpoints included OS, and secondary biomarkers included clinical and pathological risk factors, PD-L1, TMB, and molecular gene signatures from RNAseq. Formal testing in IMvigor010 of OS as the secondary endpoint was not permitted based on the hierarchical study design. The analysis plan required significance assessment for primary analyses to be made at a level of p-value<0.05. Bonferroni correction was applied to p-values for the 4 pre-specified primary objectives (5 hypotheses total).

Hazard ratios (HR) for recurrence or death were estimated using a univariable Cox proportional-hazards model. For completeness, (Tables 1, 2, and 7) we provide additional estimates for 1) stratified Cox model using the same stratification factors as described for the IMvigor010 primary clinical analysis (nodal status, PD-L1 status, and tumor stage), and 2) multivariable Cox regression analysis adjusting for nodal status, PD-L1 status, tumor stage, prior neoadjuvant chemotherapy, and number of lymph nodes resected. All Cox models used “exact” method for handling tied event times. DFS and OS were compared between treatment groups using the log-rank test, and Kaplan-Meier methodology was applied to DFS and OS with 95% CIs constructed by Greenwood's formula.

TABLE 1
DFS and OS: ctDNA(+) vs. ctDNA(−) for Atezolizumab and Observation Arms

| Model | DFS HR (95% CI) | OS HR (95% CI) | No. of patients |
|---|---|---|---|
| Atezolizumab arm: C1D1 ctDNA(+) vs. C1D1 ctDNA(−) | | | ctDNA(−): 184 |
| | | | ctDNA(+): 116 |
| | | | Total: 300 |
| Univariable* | 3.36 (2.44-4.62) | 3.63 (2.34-5.64) | |
| IMvigor010 primary† | 3.56 (2.51-5.04) | 4.19 (2.61-6.73) | |
| Multivariable^{‡} | 3.39 (2.43-4.47) | 4.08 (2.57-6.47) | |
| Observation: C1D1 ctDNA(+) vs. C1D1 ctDNA(−) | | | ctDNA(−): 183 |
| | | | ctDNA(+): 98 |
| | | | Total: 281 |
| Univariable | 6.30 (4.45-8.92) | 8.00 (4.92-12.99) | |
| IMvigor010 primary | 6.27 (4.32-9.09) | 8.08 (4.83-13.51) | |
| Multivariable | 6.19 (4.29-8.91) | 7.92 (4.81-13.05) | |
| Atezolizumab arm: C3D1 ctDNA(+) vs. C3D1 ctDNA(−) | | | ctDNA(−): 170 |
| | | | ctDNA(+): 93 |
| | | | Total: 263 |
| Univariable | 5.24 (3.68-7.45) | 6.00 (3.6-10.00) | |
| IMvigor010 primary | 5.87 (3.99-8.63) | 7.35 (4.26-12.69) | |
| Multivariable | 5.37 (3.73-7.74) | 6.94 (4.08-11.82) | |
| Observation arm: C3D1 ctDNA(+) vs. C3D1 ctDNA(−) | | | ctDNA(−): 129 |
| | | | ctDNA(+): 93 |
| | | | Total: 222 |
| Univariable | 8.65 (5.67-13.18) | 12.74 (6.26-25.93) | |
| IMvigor010 primary | 8.10 (5.15-12.76) | 12.07 (5.63-25.86) | |
| Multivariable | 8.36 (5.35-13.07) | 11.80 (5.67-24.48) | |

C1D1 ctDNA status based on C1D1 BEP. C3D1 ctDNA status based on C1/C3 BEP.
*Univariable Cox proportional-hazard model was prespecified in ctDNA statistical analysis plan.
^{†}Stratified Cox proportional-hazards model was used for IMvigor010 primary analysis. Stratification factors were: nodal status (+ or −), PD-L1 status (IC0/1 or IC2/3), and tumor stage (≤pT2 or pT3/4).
^{‡}Multivariable Cox proportional-hazards regression analysis was prespecified in ctDNA statistical analysis plan. Stratification factors were: nodal status (+ or −), PD-L1 status (IC0/1 or IC2/3), tumor stage (≤pT2 or pT3/4), prior neoadjuvant chemotherapy (yes or no), and number of lymph nodes (<10 or ≥10).

TABLE 2
DFS and OS: Atezolizumab vs. Observation Based on C1D1 ctDNA Status

| Model | DFS HR (95% CI) | OS HR (95% CI) | No. of patients |
|---|---|---|---|
| C1D1 ctDNA(+) subgroup: Atezolizumab vs. observation | | | Atezolizumab: 116 |
| | | | Observation: 98 |
| | | | Total: 214 |
| Univariable* | 0.58 (0.43-0.79) | 0.59 (0.41-0.86) | |
| IMvigor010 primary^{†} | 0.57 (0.41-0.79) | 0.58 (0.39-0.85) | |
| Multivariable^{‡} | 0.56 (0.41-0.77) | 0.58 (0.40-0.86) | |
| C1D1 ctDNA(−) subgroup: Atezolizumab vs. observation | | | Atezolizumab: 184 |
| | | | Observation: 183 |
| | | | Total: 367 |
| Univariable | 1.14 (0.81-1.62) | 1.31 (0.77-2.23) | |
| IMvigor010 primary | 1.07 (0.75-1.53) | 1.22 (0.71-2.09) | |
| Multivariable | 1.07 (0.75-1.52) | 1.22 (0.71-2.08) | |

*Univariable Cox proportional-hazard model was prespecified in ctDNA statistical analysis plan.
^{†}Stratified Cox proportional-hazards model was used for IMvigor010 primary analysis. Stratification factors were: nodal status
(+ or −), PD-L1 status (IC0/1 or IC2/3), and tumor stage (≤pT2 or pT3/4).
^{‡}Multivariable Cox proportional-hazards regression analysis was prespecified in ctDNA statistical analysis plan. Stratification factors were: nodal status (+ or −), PD-L1 status (IC0/1 or IC2/3), tumor stage (≤pT2 or pT3/4), prior neoadjuvant chemotherapy (yes or no), and number of lymph nodes (<10 or ≥10).

Descriptive statistics were used to summarize clinical characteristics, including the mean, median and range for continuous variables and frequency and percentage for categorical variables. Comparison of ctDNA clearance between arms was assessed using Fisher's Exact test (two-sided). Association between ctDNA positivity and baseline prognostic factors were measured using the Kruskal-Wallis Rank Sum test for numeric variables and Fisher's Exact test (two-sided) for categorical variables. Association between C1D1 collection time in days from surgery and ctDNA status was measured using Wilcoxon test (two-sided). All statistical analyses were performed in R.

xiv. ABACUS Trial Design

This clinical trial was not designed to be analysed concurrently with IMvigor010. The clinical aspects of ABACUS have been published previously (Powles et al. Nat Med. 25(11): 1706-1714 (2019)). This ctDNA analysis was exploratory. The methods of the trial are summarized briefly as follows: This study was an open-label, international, multicenter phase II trial, evaluating the efficacy of two cycles (1200 mg Q3W) of preoperative atezolizumab in patients with histologically confirmed (T2-T4a) urothelial bladder cancer awaiting planned cystectomy. The design endpoints and inclusion criteria have been published previously (Powles et al. Nat Med. 25(11): 1706-1714 (2019)). Briefly, eligibility criteria included MIBC patients who refused or were not able to have cisplatin-based neoadjuvant chemotherapy, had no evidence of advanced disease, ECOG Performance Status of 0 or 1, and adequate end-organ function. Major exclusion criteria included prior use of immune checkpoint inhibitors and contraindications for immune therapy or cystectomy. All patients provided written informed consent, which included the exploratory biomarker endpoints described here. The study was approved by the relevant institutional review board and ethics committee for each participating center and was conducted in accordance with the principles of Good Clinical Practice, the provisions of the Declaration of Helsinki, and other applicable local regulations (NCT02662309). The study was sponsored by Queen Mary University of London. The Barts Experimental Cancer Centre Clinical Trials Group had overall responsibility for trial management and day-to-day running of the trial, and the trial was overseen by an independent data monitoring committee (IDMC). Emerging safety data was reviewed regularly by the IDMC.

D. Results

i. IMvigor010 ctDNA Biomarker Evaluable Population

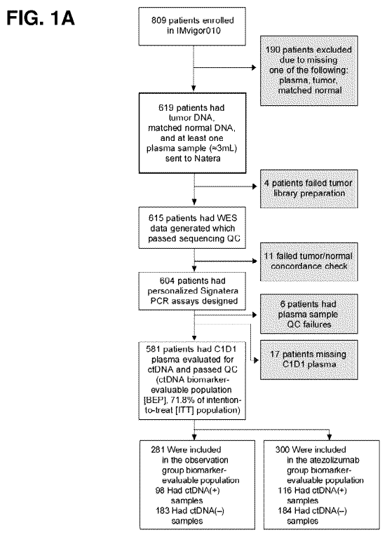

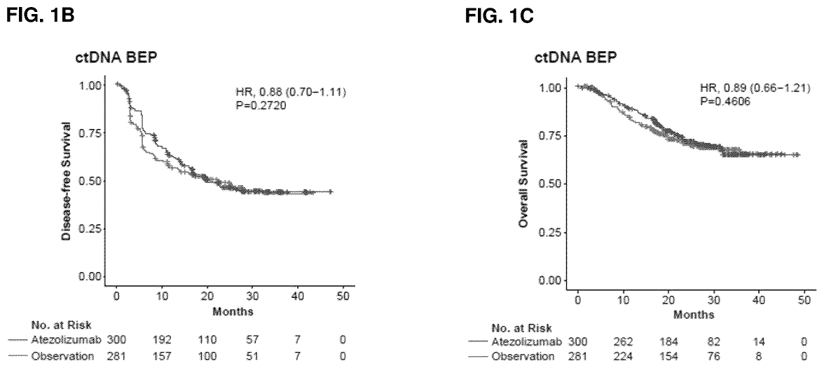

A total of 809 patients were enrolled in the IMvigor010 study (406 atezolizumab arm; 403 observation arm), with a median follow up of 21.9 months. There were 581 patients included in the ctDNA C1 D1 BEP (72% of the ITT population), with a median follow up of 23.0 months (FIG. 1A). Baseline characteristics of the ctDNA BEP population were comparable and well balanced between arms (Table 3), and survival outcomes were as described for DFS (HR=0.88 (0.70-1.11); p=0.2720) (FIG. 1B) and OS (HR=0.89 (0.66-1.21)) (FIG. 1C).

TABLE 3
Comparison of Baseline Characteristics in the Cycle
1 Day 1 ctDNA Biomarker-Evaluable Population (BEP)

| | Atezolizumab | Observation |
|---|---|---|
| | (n = 300) | (n = 281) |
| Median age (range) - yr | 67 (31-85) | 66 (37-88) |

Race - no. (%)

| | | |
|---|---|---|
| White | 242 (80.7%) | 220 (78.3%) |
| Asian | 42 (14.1%) | 42 (15.0%) |
| Black or African American | 2 (0.7%) | 1 (0.4%) |
| Other | 6 (2.0%) | 3 (1.1%) |
| Unknown | 7 (2.3%) | 15 (5.3%) |

Sex - no. (%)

| | | |
|---|---|---|
| Female | 67 (22.3%) | 62 (21.4%) |
| Male | 233 (77.7%) | 221 (78.7%) |

Baseline Eastern Cooperative Oncology Group
performance score - no. (%)

| | | |
|---|---|---|
| 0 | 188 (62.7%) | 183 (65.1%) |
| 1 | 99 (33.0%) | 88 (31.3%) |
| 2 | 13 (4.3%) | 10 (3.6%) |

Primary tumor site - no. (%)

| | | |
|---|---|---|
| Muscle-invasive bladder cancer | 278 (92.7%) | 260 (92.5%) |
| Upper tract urothelial carcinoma | 22 (7.3%) | 21 (7.4%) |

Tumor stage - no. (%)

| | | |
|---|---|---|
| <PT2/PT2 | 77 (25.7%) | 71 (25.3%) |
| PT3/PT4 | 223 (74.3%) | 210 (74.7%) |

Prior neoadjuvant or adjuvant treatment - no. (%)

| | | |
|---|---|---|
| No | 154 (51.3%) | 147 (52.3%) |
| Yes | 146 (48.7%) | 134 (47.7%) |

PD-L1 status* - no. (%)

| | | |
|---|---|---|
| IC0/1: PD-L1(−) | 160 (53.3%) | 145 (51.9%) |
| IC2/3: PD-L1(+) | 140 (46.7%) | 136 (48.1%) |

Lymph nodes - no. (%)

| | | |
|---|---|---|
| <10 | 65 (21.7%) | 62 (22.1%) |
| ≥10 | 235 (78.3%) | 219 (77.9%) |

Nodal status - no. (%)

| | | |
|---|---|---|
| Negative | 145 (48.3%) | 133 (47.3%) |
| Positive | 155 (51.7%) | 148 (52.7%) |
| Median TMB (range), mut/mb | 6.75 (0.51-52.73) | 7.02 (0.41-73.52) |

*Per VENTANA SP142 immunohistochemistry assay.
†Eighty-five patients had missing data.
‡One hundred nine patients had missing data.

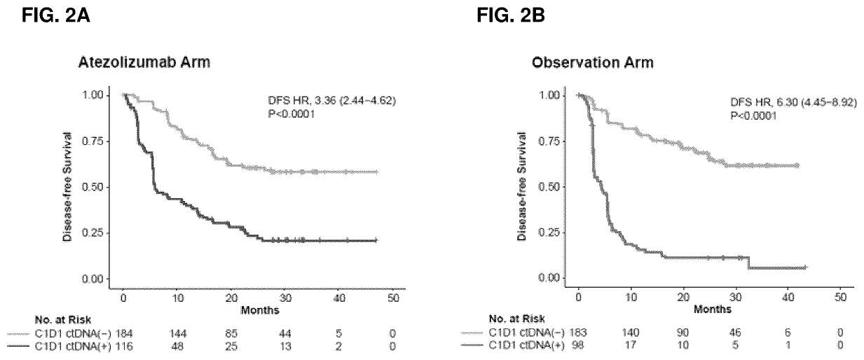

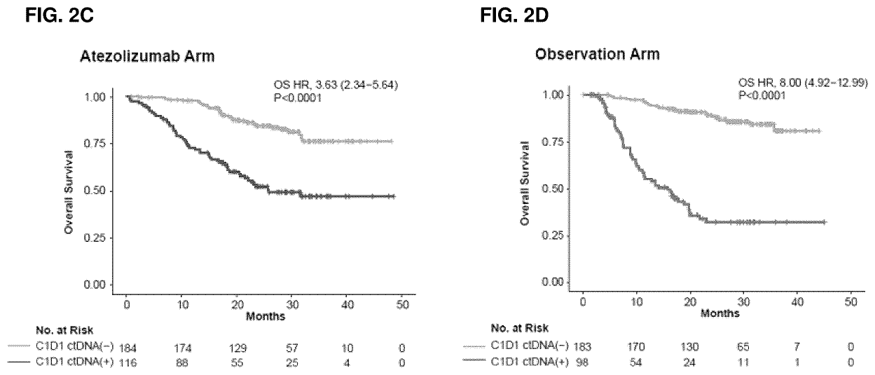

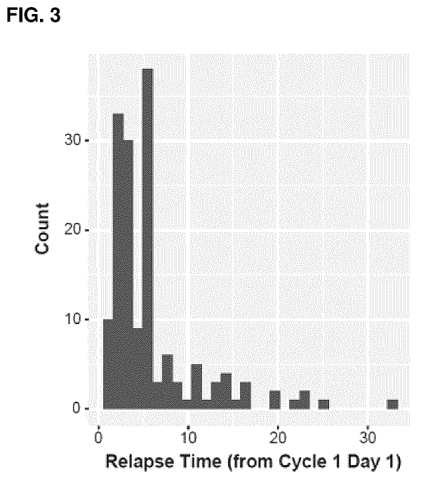

At C1 D1, it was found that 37% (214/581) of patients were ctDNA(+). ctDNA positivity identified patients at higher risk of disease recurrence compared to ctDNA(−) (observation arm DFS HR=6.3 (4.45-8.92); p<0.0001), and shorter OS (observation arm HR=8.0 (4.92-12.99)) (FIGS. 2B and 2D). In the C1D1 ctDNA(+) population, there were 116 patients in the atezolizumab arm and 98 in the observation arm, and baseline characteristics including immune biomarkers were balanced across arms (Table 4). Analyses were repeated using a multivariable approach and the results were similar (Table 1). C1 D1 collection time after surgery (median 79 days) did not associate with higher rates of ctDNA positivity or higher ctDNA levels (FIGS. 16A-16D). No difference was found between the collection times for the ctDNA-negative patients and ctDNA-positive patients (Wilcoxon p=0.18, two sided). ctDNA positivity at C1D1 preceded clinical relapse by radiological imaging by a median of 4.3 months (range 0.7-32.3 months) (FIG. 3).

TABLE 4
Balance of Baseline Characteristics Between
Arms Within ctDNA(+) Population

| | Atezolizumab | Observation |
|---|---|---|
| | (n = 116) | (n = 98) |
| Median age (range) - yr | 67 (32-83) | 68 (37-88) |

Race - no. (%)

| | | |
|---|---|---|
| Asian | 16 (13.8%) | 11 (11.2%) |
| Other | 1 (0.9%) | 2 (2.0%) |
| Unknown | 4 (3.4%) | 8 (8.2%) |
| White | 95 (81.9%) | 77 (78.6%) |

Sex - no. (%)

| | | |
|---|---|---|
| Female | 28 (24.1%) | 24 (24.5%) |
| Male | 88 (75.9%) | 74 (75.5%) |

Baseline Eastern Cooperative Oncology Group
performance score - no. (%)

| | | |
|---|---|---|
| 0 | 71 (61.2%) | 64 (65.3%) |
| 1 | 36 (31.0%) | 31 (31.6%) |
| 2 | 9 (7.8%) | 3 (3.1%) |

Primary tumor site - no. (%)

| | | |
|---|---|---|
| Muscle-invasive bladder cancer | 106 (91.4%) | 93 (94.9%) |
| Upper tract urothelial carcinoma | 10 (8.6%) | 5 (5.1%) |

Tumor stage - no. (%)NA's

| | | |
|---|---|---|
| <PT2/PT2 | 24 (20.7%) | 22 (22.4%) |
| PT3/PT4 | 92 (79.3%) | 76 (77.6%) |

Prior neoadjuvant therapy - no. (%)

| | | |
|---|---|---|
| NO | 64 (55.2%) | 47 (48.0%) |
| YES | 52 (44.8%) | 51 (52.0%) |

PD-L1 status* - no. (%)

| | | |
|---|---|---|
| IC0/1: PD-L1(−) | 54 (46.6%) | 58 (59.2%) |
| IC2/3: PD-L1(+) | 62 (53.4%) | 40 (40.8%) |

Lymph nodes - no. (%)

| | | |
|---|---|---|
| <10 | 25 (21.6%) | 22 (22.4%) |
| ≥10 | 91 (78.4%) | 76 (77.6%) |

Nodal status - no. (%)

Negative 35 (30.2%) 35 (35.7%)

| | | |
|---|---|---|
| Positive | 81 (69.8%) | 63 (64.3%) |

TMB status

| | | |
|---|---|---|
| TMB_high | 37 (31.9%) | 32 (32.7%) |
| TMB_low | 79 (68.1%) | 66 (67.3%) |

| | |
|---|---|
| Atezolizumab | Observation |
| (n = 114) | (n = 98) |

tGE3 - no. (%)

| | | |
|---|---|---|
| High | 63 (55.3%) | 43 (43.9%) |
| Low | 51 (44.7%) | 55 (56.1%) |

TBRS - no. (%)

| | | |
|---|---|---|
| High | 52 (45.6%) | 51 (52.0%) |
| Low | 62 (54.4%) | 47 (48.0%) |

TCGA subtype - no. (%)

| | | |
|---|---|---|
| Basal-squamous | 44 (38.6%) | 32 (32.7%) |
| Luminal | 12 (10.5%) | 11 (11.2%) |
| Luminal-infiltrated | 24 (21.1%) | 32 (32.7%) |
| Luminal-papillary | 32 (28.1%) | 22 (22.4%) |
| Neuronal | 2 (1.8%) | 1 (1.0%) |

Angiogenesis - no. (%)

| | | |
|---|---|---|
| High | 54 (47.4%) | 50 (51.0%) |
| Low | 60 (52.6%) | 48 (49.0%) |

*Per VENTANA SP142 immunohistochemistry assay.
NA, not available.

ii. ctDNA Positivity at C1D1 was Associated with Improved DFS on Atezolizumab Compared to Observation

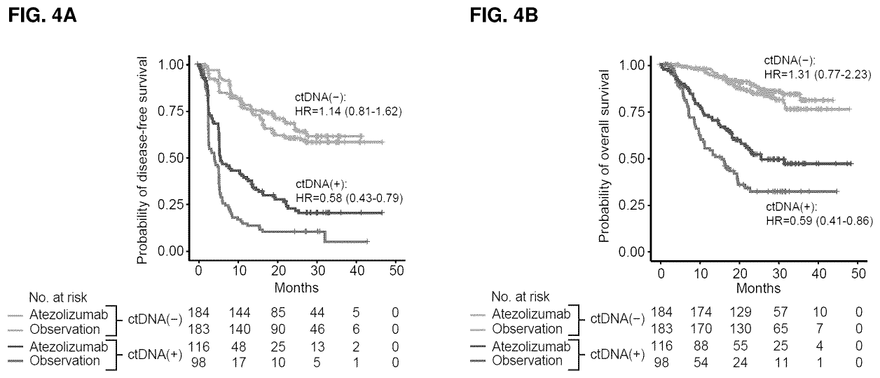

Patients who were ctDNA(+) at C1D1 had improved DFS on adjuvant atezolizumab compared to patients receiving observation (HR=0.58 (0.43-0.79); p=0.0024, median DFS 4.4 vs. 5.9 months) (FIG. 4A). Similarly, this ctDNA(+) patient population had improved OS with atezolizumab as compared to observation (HR=0.59 (0.41-0.86); median OS 15.8 vs. 25.8 months) (FIG. 4B). No difference in DFS or OS between arms was found for patients who were ctDNA negative (ctDNA(−)) (HR=1.14 (0.81-1.62); and HR=1.31 (0.77-2.23), respectively (median not reached in either population)). Analyses were repeated using a multivariate approach and the results were similar (Table 2).

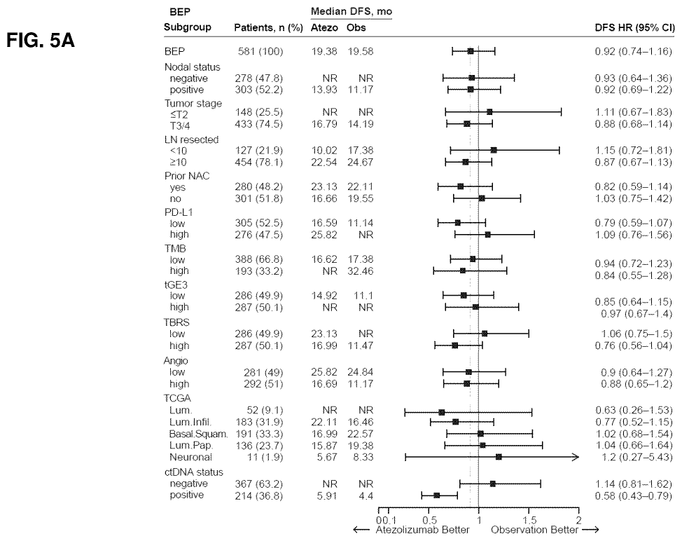

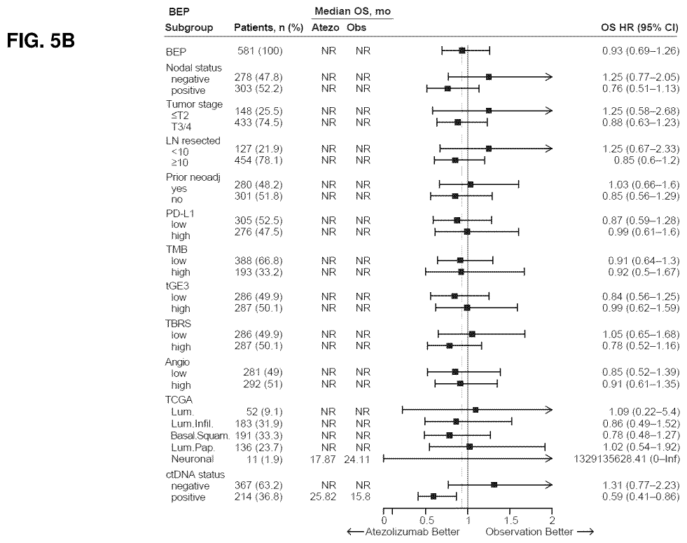

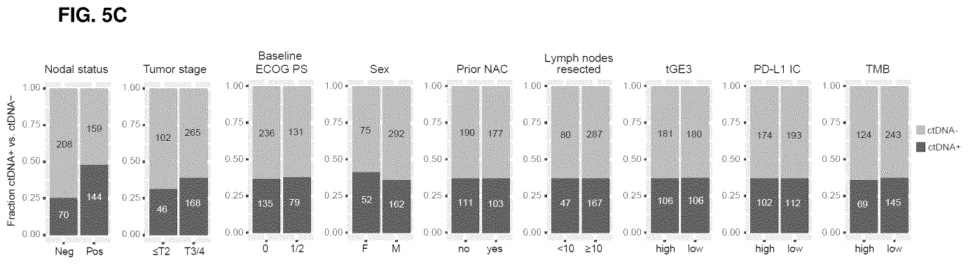

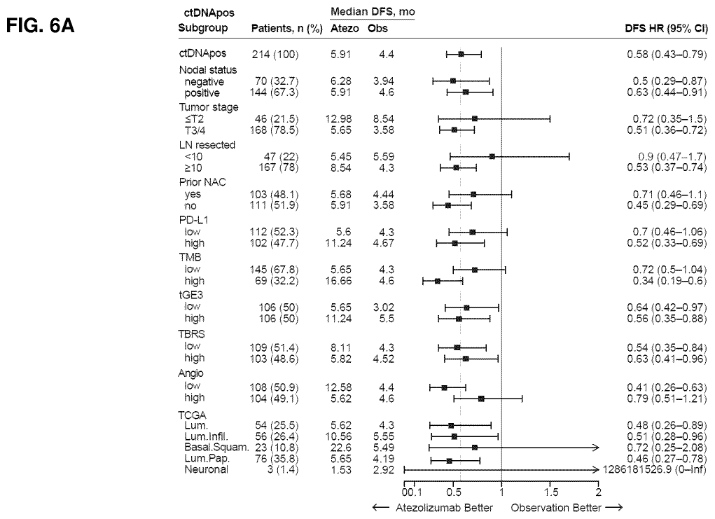

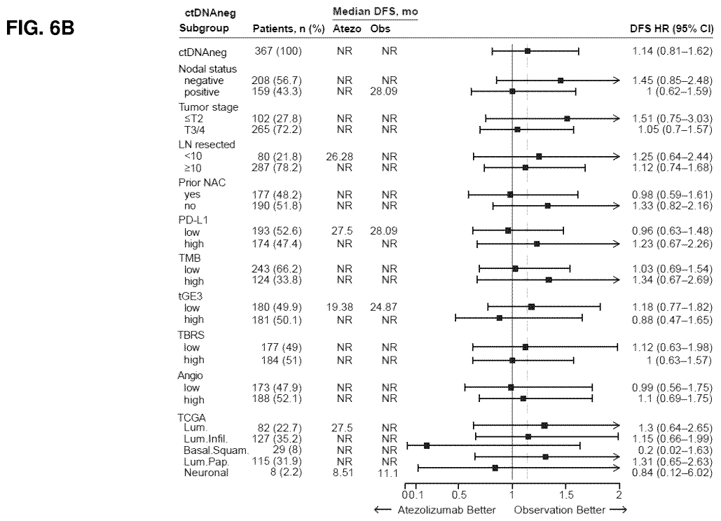

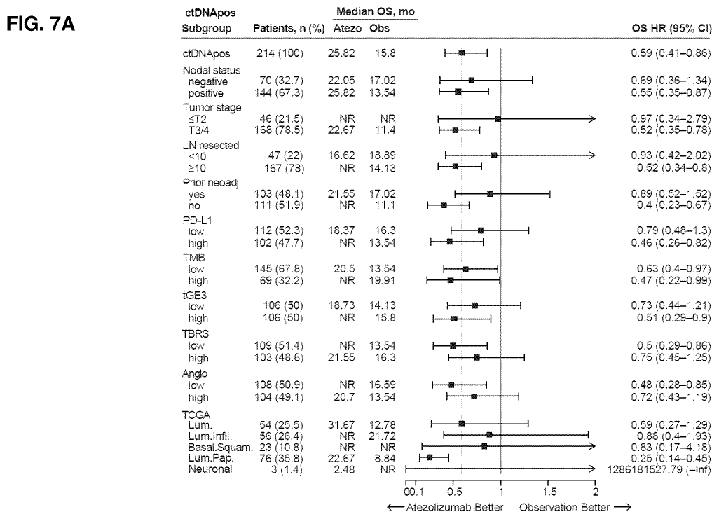

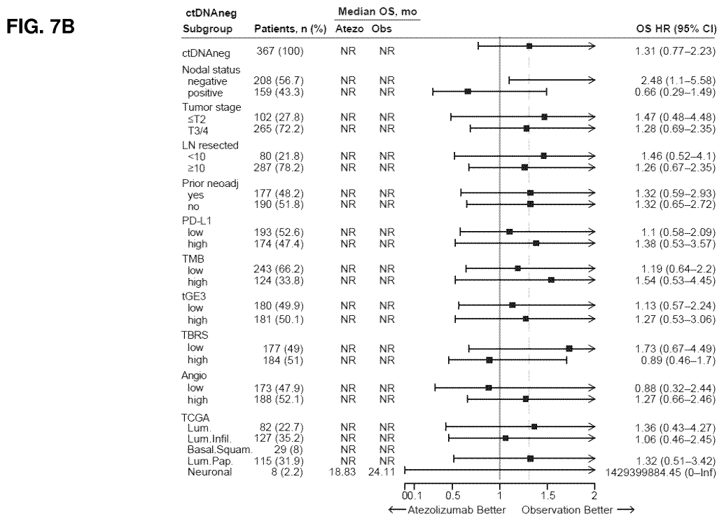

To assess whether other important baseline clinical factors were driving these results, exploratory analysis was performed on features at baseline including nodal status, tumor stage, prior neoadjuvant chemotherapy, PD-L1 status, and number of lymph nodes resected. Univariable analysis in the biomarker evaluable population did not find subgroups with improved outcomes on atezolizumab (FIGS. 5A-5C). Furthermore, adjusting for these clinical features in a multivariable analysis of DFS and OS confirmed that ctDNA can independently identify patients with improved outcomes to atezolizumab (Tables 1, 2, and 6). Lastly, subgroups within the ctDNA(+) population showed no clear evidence that a single clinical feature was driving the improved outcomes seen in the ctDNA(+) patients (FIGS. 6A, 6B, 7A, and 7B).

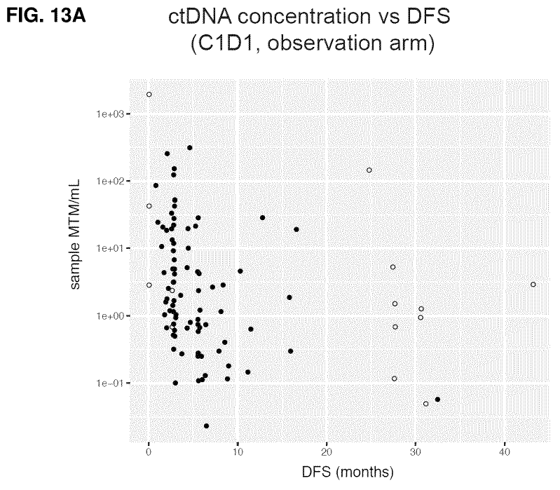

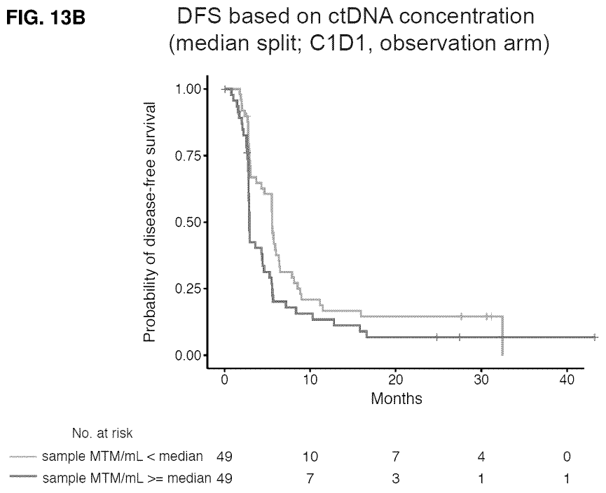

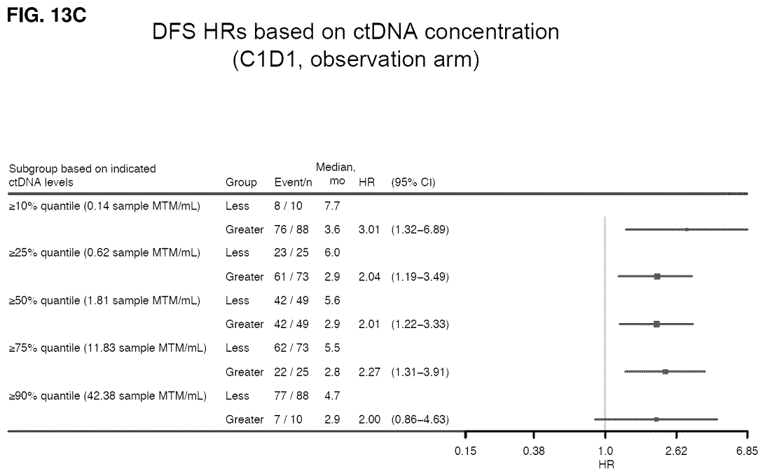

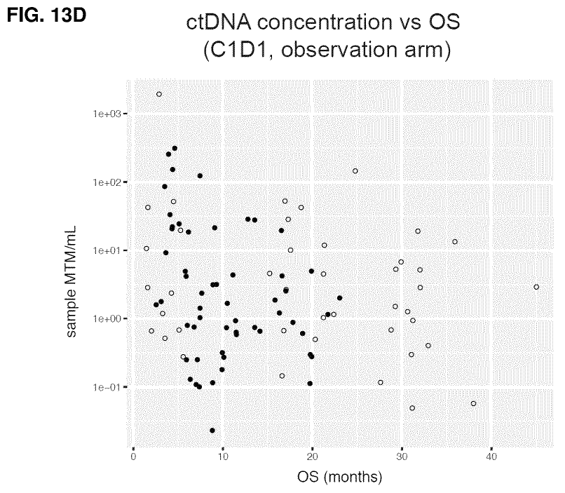

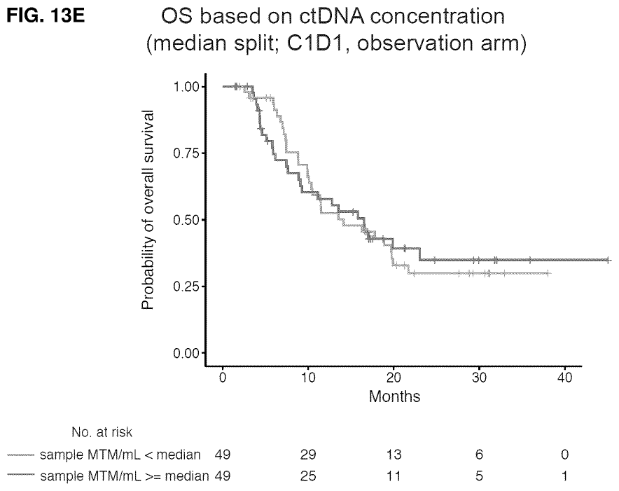

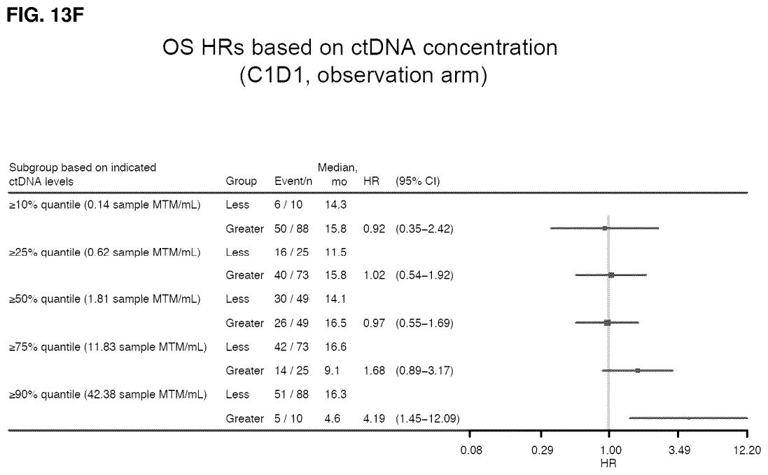

To support the findings that used binary cutoffs for ctDNA, continuous ctDNA metrics were also evaluated as a secondary exploratory objective. Higher thresholds of the sample MTM/mL (sample mean tumor molecules per mL of plasma) did not identify a group at substantially higher risk of relapse or death (FIGS. 13A-13F), suggesting that any presence of ctDNA is more relevant than the total burden of ctDNA in identifying high-risk patients.

iii. Improved DFS in ctDNA(+)/TMB(+) Patients and ctDNA(+)/PD-L1(+) Patients

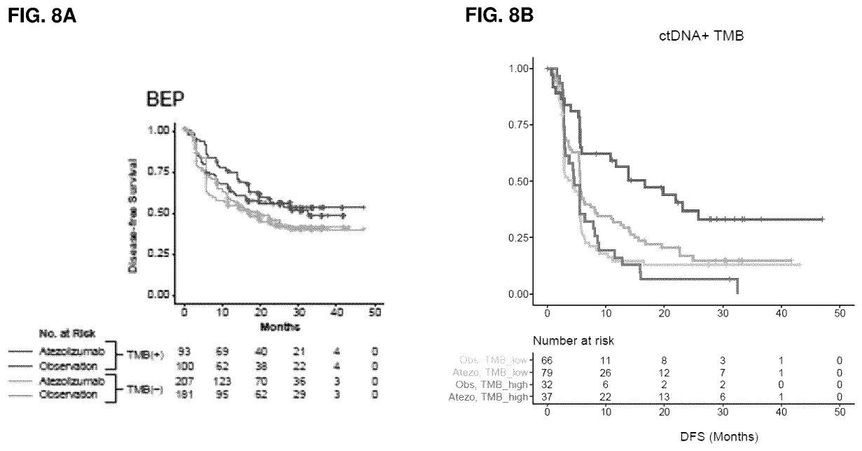

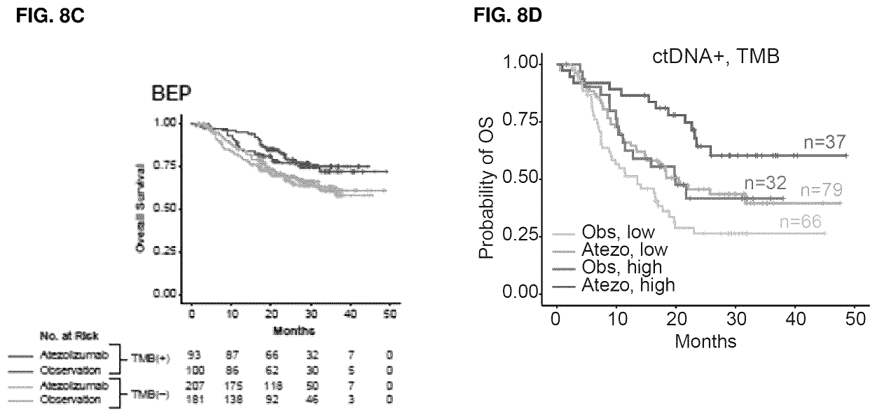

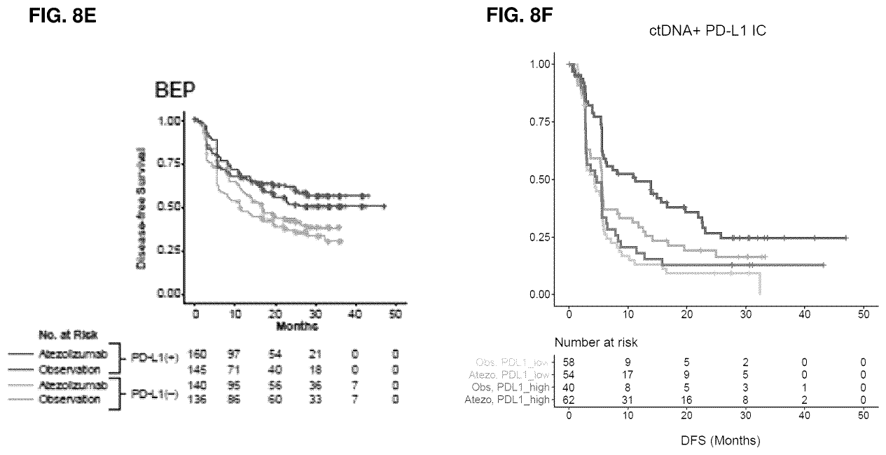

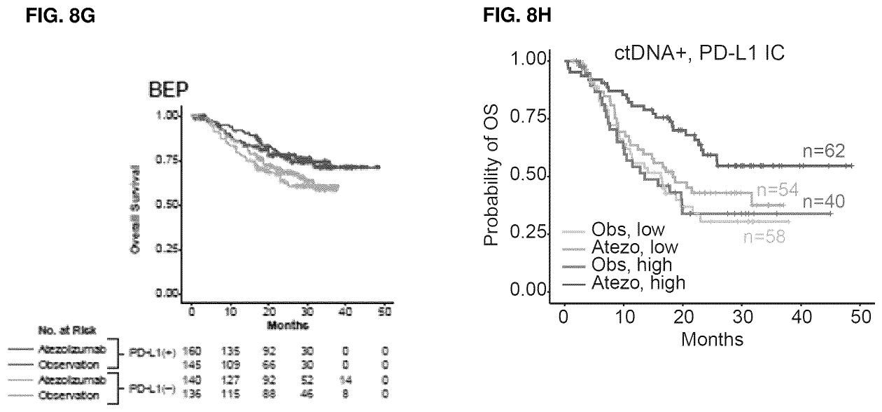

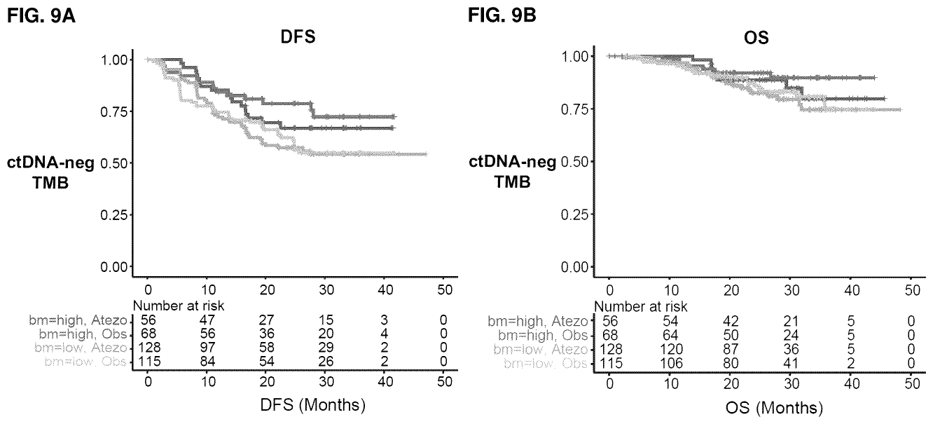

Across all patients in the biomarker study (regardless of ctDNA status), high TMB (TMB+) was not predictive of DFS benefit from atezolizumab (HR=0.84 (0.55-1.28)) (FIGS. 8A, 9A, and 9B). However, ctDNA(+)/TMB(+) patients showed improved DFS hazard ratio (HR=0.34 (0.19-0.6)) compared to ctDNA(+)/TMB(−) (HR=0.72 (0.50-1.04)) (FIG. 8B). Similar findings were observed when OS was measured in the same population (ctDNA(+)/TMB(+) HR=0.47 (0.22-0.99) vs. ctDNA(+)/TMB(−) HR=0.63 (0.4-0.97)) (FIGS. 8C and 8D), as well as when a multivariate approach was used.

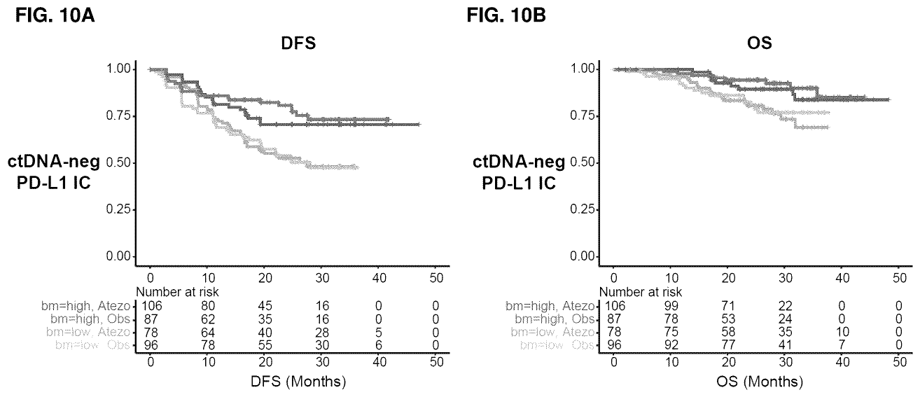

Similarly, PD-L1 high (PD-L1+) status did not enrich for DFS benefit in the biomarker study population (regardless of ctDNA status) (HR=1.09 (0.76-1.56)) (FIGS. 8E, 10A, and 10B). However, ctDNA(+)/PD-L1(+) showed improved DFS hazard ratio (HR=0.52 (0.33-0.82)) compared to ctDNA(+)/PD-L1(−) (HR=0.70 (0.46-1.06)) (FIG. 8F). Similar findings were observed when OS was measured in the same population (ctDNA(+)/PD-L1(+) HR=0.46 (0.26-0.82) vs. ctDNA(+)/PD-L1(−) HR=0.79 (0.48-1.30)) (FIGS. 8G and 8H). A multivariate approach gave similar results.

In order to evaluate changes in ctDNA status in response to treatment, patients with plasma samples from both C1D1 and C3D1 were studied (485 patients, 60% of ITT). This C1D1/C3D1 BEP was analyzed for imbalances between the atezolizumab and observation arms and clinical factors. Baseline characteristics were generally well balanced, and no imbalances were found (Table 5).

TABLE 5
Comparison of Baseline Characteristics in the C1/C3 BEP*

| | Atezolizumab | Observation |
|---|---|---|
| | (n = 263) | (n = 222) |
| Median age (range) - yr | 67 (31-85) | 66 (37-88) |

Race - no. (%)

| | | |
|---|---|---|
| White | 208 (79.1%) | 169 (76.1%) |
| Non-White | 55 (20.9%) | 53 (23.9%) |

Sex - no. (%)

| | | |
|---|---|---|
| Female | 60 (22.8%) | 51 (23.0%) |
| Male | 203 (77.2%) | 171 (77.0%) |

Baseline Eastern Cooperative Oncology
Group performance score - no. (%)

| | | |
|---|---|---|
| 0 | 168 (63.9%) | 147 (66.2%) |
| 1 | 85 (32.3%) | 66 (29.7%) |
| 2 | 10 (3.8%) | 9 (4.1%) |

Primary tumor site - no. (%)

| | | |
|---|---|---|
| Muscle-invasive bladder cancer | 244 (92.8%) | 204 (91.9%) |
| Upper tract urothelial carcinoma | 19 (7.2%) | 18 (8.1%) |

Tumor stage - no. (%)

| | | |
|---|---|---|
| <PT2/PT2 | 70 (26.6%) | 56 (25.2%) |
| PT3/PT4 | 193 (73.38%) | 166 (74.77%) |

Prior neoadjuvant or adjuvant
treatment - no. (%)

| | | |
|---|---|---|
| No | 134 (51.0%) | 115 (51.8%) |
| Yes | 129 (49.1%) | 107 (48.2%) |

PD-L1 status† - no. (%)

| | | |
|---|---|---|
| IC0/1: PD-L1(−) | 143 (54.4%) | 116 (52.3%) |
| IC2/3: PD-L1(+) | 120 (45.6%) | 106 (47.8%) |

Lymph nodes - no. (%)

| | | |
|---|---|---|
| <10 | 55 (20.9%) | 48 (21.6%) |
| ≥10 | 208 (79.1%) | 174 (78.4%) |

Nodal status - no. (%)

| | | |
|---|---|---|
| Negative | 128 (48.7%) | 101 (45.5%) |
| Positive | 135 (51.3%) | 121 (54.5%) |

*Patients had ctDNA samples at C1 and C3.
†Per VENTANA SP142 immunohistochemistry assay.

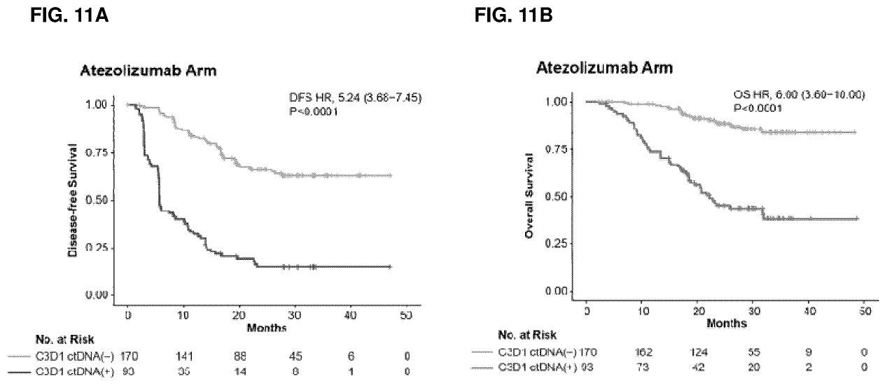

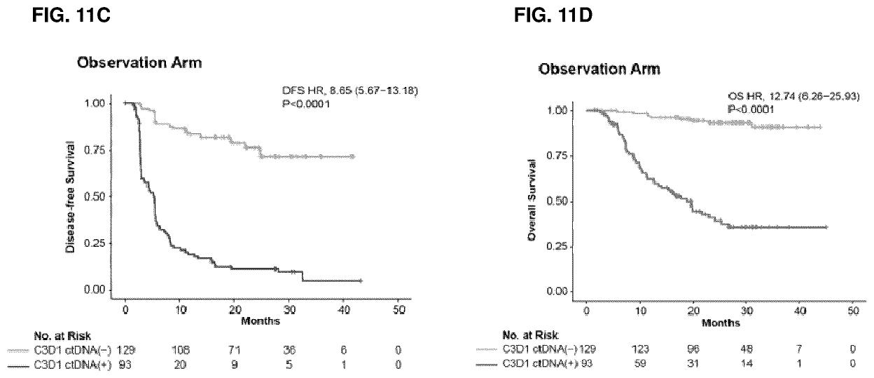

At C3D1, it was found that 38.4% (188/485) of patients were ctDNA(+), and these patients were at higher risk for disease progression and relapse compared to ctDNA(−) (observation arm DFS HR=8.65 (5.67-13.18); p<0.0001) (FIGS. 11A-11D). C3D1 ctDNA positivity was also a negative prognostic factor for OS (observation arm OS HR=12.74 (6.26-25.93); p<0.0001). Results were similar when using a multivariate approach (Table 1).

iv. Changes in ctDNA Status from Baseline (C1D1) to On-Treatment (C3D1) Time Point; ctDNA Clearance was Associated with Improved DFS

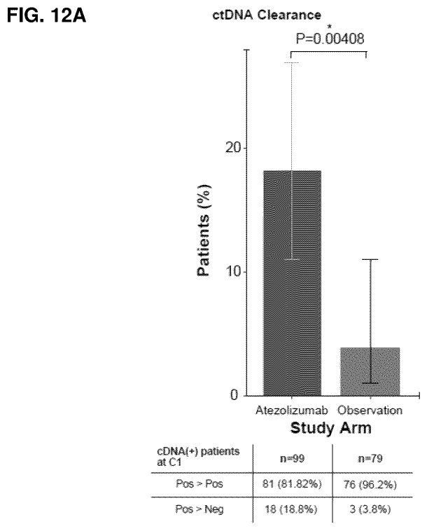

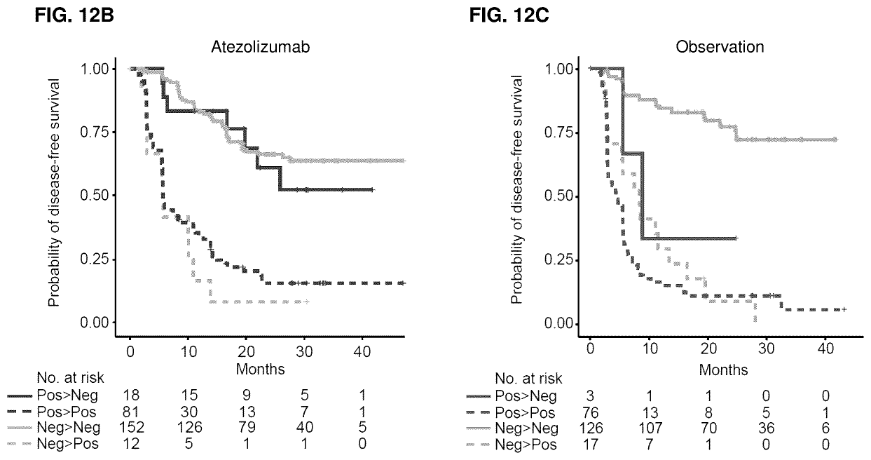

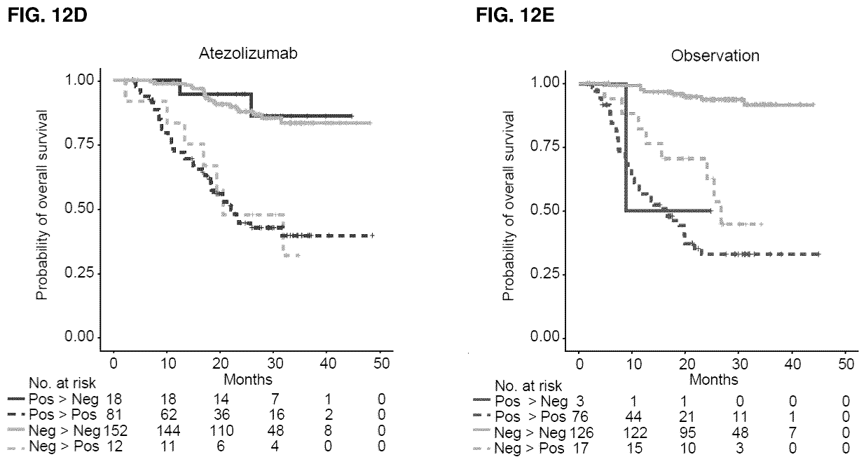

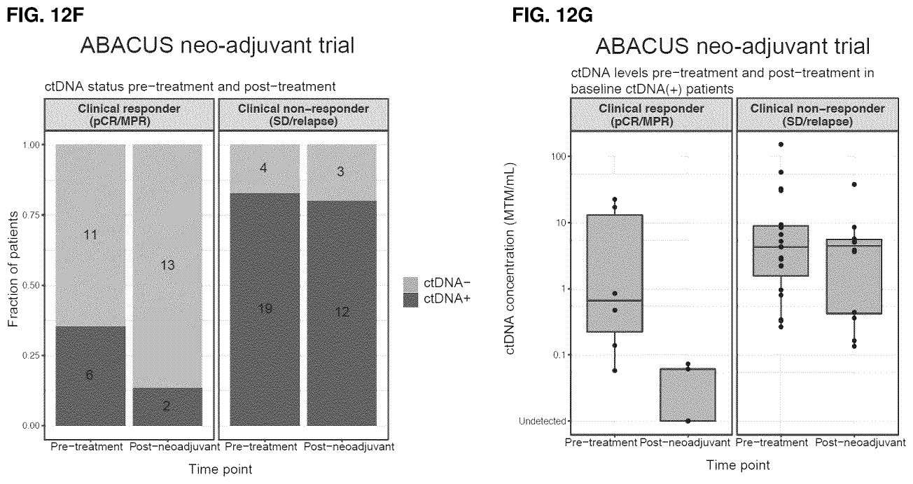

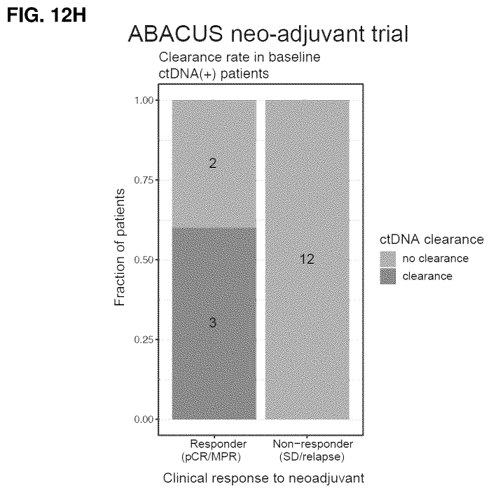

ctDNA clearance, assessed in patients who were ctDNA(+) at C1D1 and defined as achieving ctDNA(−) status by C3D1, was quantified and compared between treatment arms. Clearance occurred in patients who were subsequently ctDNA(−) by C3D1, and non-clearance occurred in patients who remained ctDNA(+) at C3D1. Clearance was observed in 18.2% (18/99) of patients in the atezolizumab arm compared to 3.8% (3/79) in the observation arm (p=0.0204) (FIG. 12A). Patients who cleared ctDNA within the atezolizumab arm had superior DFS and OS compared to those who remained positive for ctDNA (DFS HR=0.26 (0.12-0.56); p=0.0014; median DFS 5.7 months versus not reached; and OS HR=0.14 (0.03-0.59)) (FIGS. 12B-12E and Table 6). Similar findings were observed when using a univariate approach (Table 7). Overall, patents who were ctDNA(−) at both time points or cleared ctDNA had longer DFS than patients who were ctDNA(+) at both time points or who became ctDNA(+) (FIGS. 12A-12E).

TABLE 6
Median DFS and OS Based on Change in ctDNA Status
from Baseline (C1D1) to On-Treatment (C3D1) Time
Point for Atezolizumab and Observation Arms

| | | |
|---|---|---|
| | Median DFS | Median OS |
| Change in ctDNA status | (95% CI) | (95% CI) |

Atezolizumab Arm

| | | |
|---|---|---|
| Neg > Neg | Not reached | Not reached |
| Pos > Neg (clearance) | Not reached | Not reached |
| Pos > Pos (non-clearance) | 5.7 (5.5-10.8) | 22.1 (18.2-NE) |
| Neg > Pos | 5.7 (2.9-10.1) | 20.6 (16.9-NE) |

Observation Arm

| | | |
|---|---|---|
| Neg > Neg | Not reached | Not reached |
| Pos > Neg (clearance) | 8.8 (5.5-NE) | 8.8 (8.8-NE) |
| Pos > Pos (non-clearance) | 4.4 (2.9-5.5) | 16.3 (10.4-19.9) |
| Neg > Pos | 8.3 (3-13.4) | 26.8 (15.5-NE) |

ctDNA dynamics from C1D1 to C3D1 including patients who were ctDNA(+) at C1D1 and cleared ctDNA by C3D1 (Pos > Neg), patients who were ctDNA(+) at C1D1 and did not clear ctDNA (Pos > Pos), patients who were ctDNA(−) at C1D1 and remained ctDNA(−) at C3D1 (Neg > Neg), and patients who were ctDNA(−) at C1D1 and became ctDNA(+) at C3D1 (Neg > Pos), for median DFS and OS in the atezolizumab arm, and median DFS and OS in the observation arm.

TABLE 7
DFS and OS: ctDNA Clearance vs. Non-Clearance
for Atezolizumab and Observation Arms

| Model | DFS HR (95% CI) | OS HR (95% CI) | No. of patients |
|---|---|---|---|
| Atezoliuzmab arm: Clearance vs. no clearance | | | Clearance: 18 |
| | | | No clearance: 81 |
| | | | Total: 99 |
| Univariable* | 0.26 (0.12-0.56) | 0.41 (0.1-1.70) | |
| IMvigor010 primary† | 0.35 (0.16-0.78) | 0.51 (0.12-2.19) | |
| Multivariable‡ | 0.32 (0.14-0.71) | 0.42 (0.10-1.79) | |
| Observation arm: Clearance vs. no clearance | | | Clearance: 3 |
| | | | No clearance: 76 |
| | | | Total: 79 |
| Univariable | 0.14 (0.03-0.59) | 0.66 (0.09-4.81) | |
| IMvigor010 primary | 0.17 (0.04-0.73) | 1.15 (0.14-9.41) | |
| Multivariable | 0.17 (0.04-0.72) | 1.17 (0.15-9.08) | |

Analysis based on patients with C1D1 ctDNA(+) status.
*Univariable Cox proportional-hazard model was prespecified in ctDNA statistical analysis plan.
†Stratified Cox proportional-hazards model was used for IMvigor010 primary analysis. Stratification factors were: nodal status (+ or −), PD-L1 status (IC0/1 or IC2/3), and tumor stage (≤pT2 or pT3/4).
‡Multivariable Cox proportional-hazards regression analysis was prespecified in ctDNA statistical analysis plan. Stratification factors were: nodal status (+ or −), PD-L1 status (IC0/1 or IC2/3), tumor stage (≤pT2 or pT3/4), prior neoadjuvant chemotherapy (yes or no), and number of lymph nodes (<10 or ≥10).

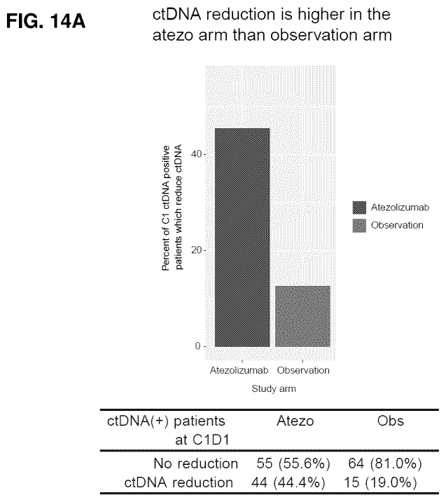

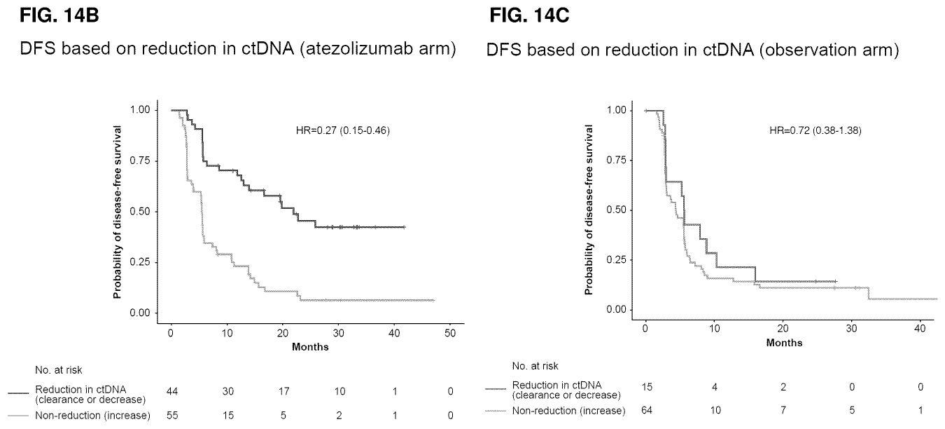

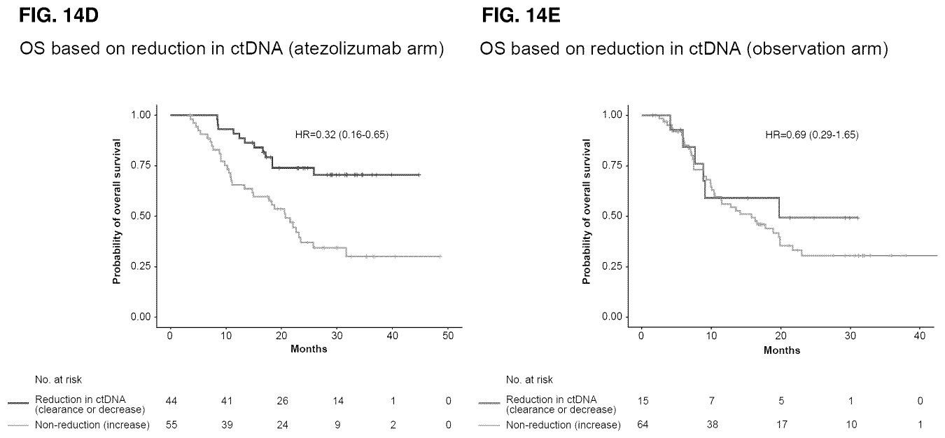

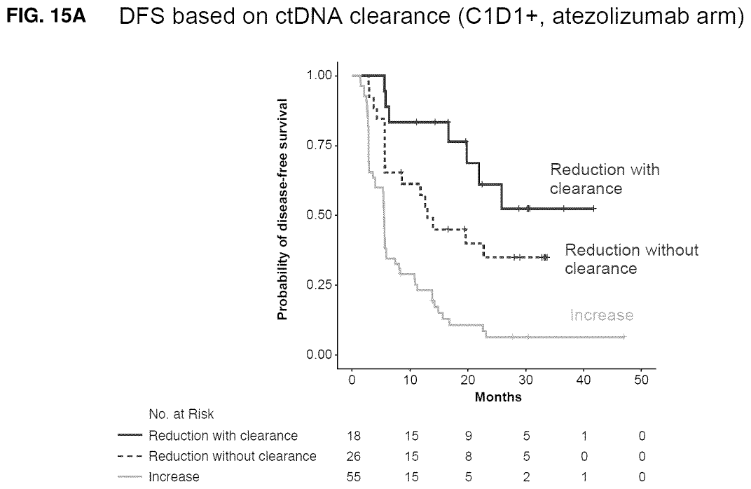

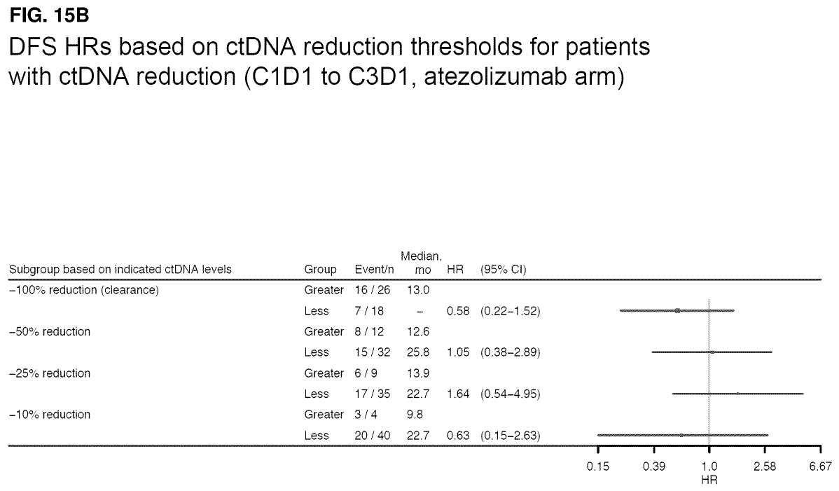

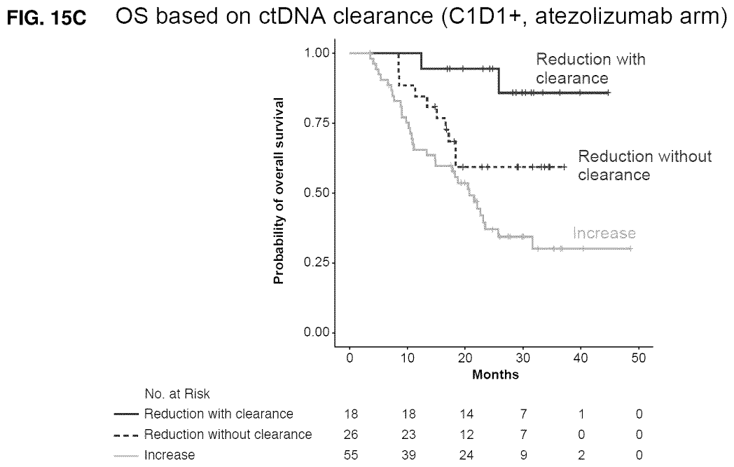

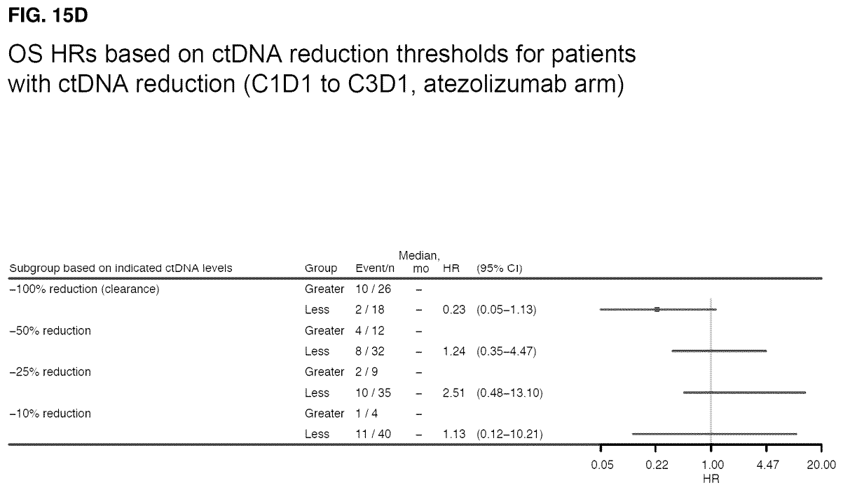

Comparing patients that have a reduction in ctDNA levels to those that have an increase, a higher frequency of patients with ctDNA reduction in the atezolizumab arm was found (44.4% versus 19.0% in observation). Reductions in ctDNA were associated with improved outcomes (FIGS. 14A-14E). The DFS/OS improvement for patients who reduce ctDNA but remain ctDNA(+) was not as pronounced as is achieved by clearance of ctDNA (FIGS. 15A-15D).

v. ABACUS ctDNA Study Supported that ctDNA Associates with Clinical Outcomes in Neoadjuvant Setting

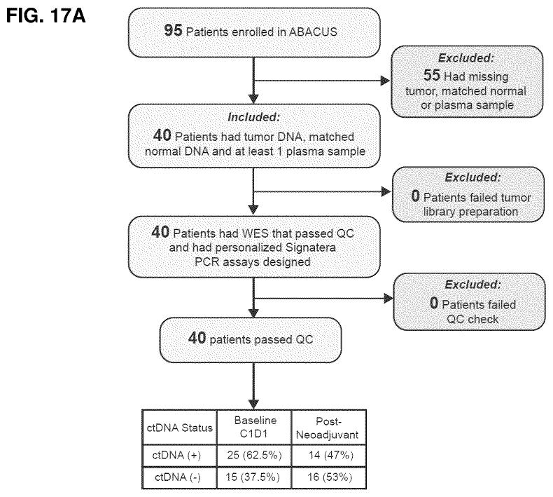

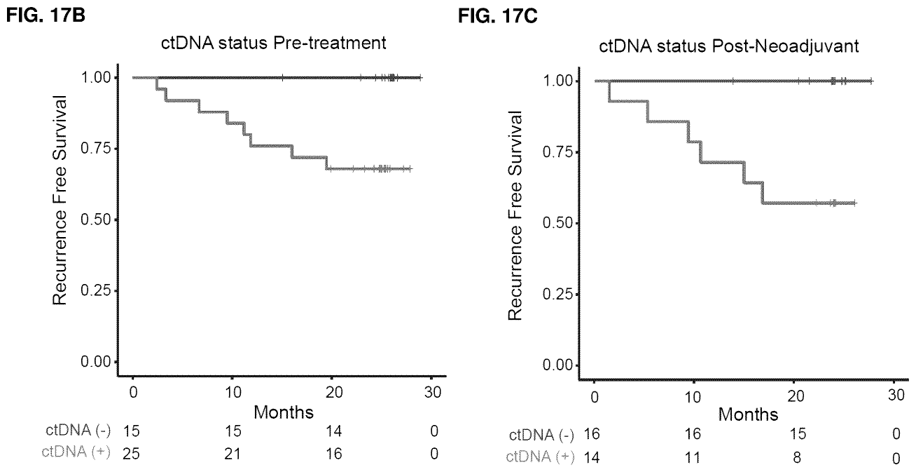

To support the findings of the work described above, we explored ctDNA data from a prospective phase II study of neoadjuvant atezolizumab prior to cystectomy in muscle invasive urothelial cancer (FIGS. 17A-17C). Clinical characteristics of the patients and the efficacy endpoints of the study have been published previously (Powles et al. Nat Med. 25(11): 1706-1714 (2019)). Briefly, 2 cycles of 3-weekly atezolizumab were given, followed by cystectomy. The study met its primary endpoint of pathological complete response, ctDNA analysis was exploratory. 40/96 patients had plasma samples available at baseline (pre-neoadjuvant) for ctDNA analysis. Samples were taken prior to and after neoadjuvant atezolizumab (pre-cystectomy). Identical ctDNA methodology was used in both studies, although concurrent analysis with IMvigor010 was not pre-specified and therefore results should be interpreted with caution. At baseline 62.5% (25/40) of patients were ctDNA(+), which correlated with a poor outcome (FIGS. 17A-17C). Atezolizumab was associated with reduction in ctDNA levels in patients who achieved pathological complete response (pCR) or major pathological response (MPR) (FIGS. 12F-12G). Clearance was assessed in patients who were ctDNA(+) at baseline and had post-neoadjuvant plasma available (n=17). Atezolizumab was associated with ctDNA clearance in 3/17 (18%) patients (FIG. 12H). Non-responding patients did not show marked changes in ctDNA levels. These results in the neoadjuvant setting further support a link between ctDNA dynamics and clinical response to atezolizumab. Therefore, these data indicate that ctDNA positivity may be useful as a predictive treatment marker of atezolizumab response in the neoadjuvant setting.

vi. Transcriptional Correlates of ctDNA Positivity, and Biomarkers for Response to Atezolizumab within the ctDNA(+) Population

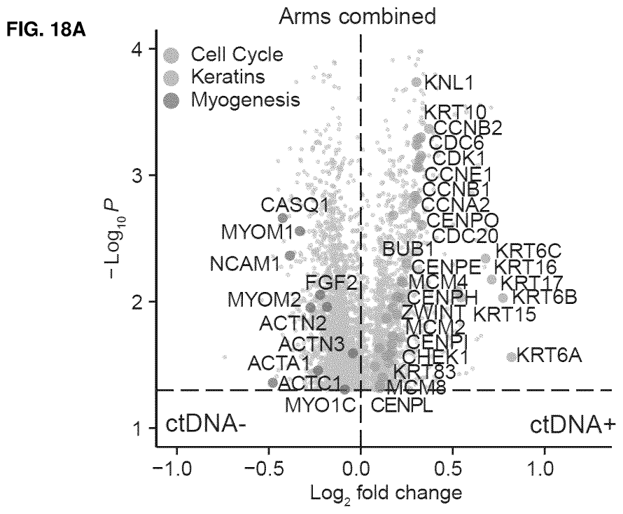

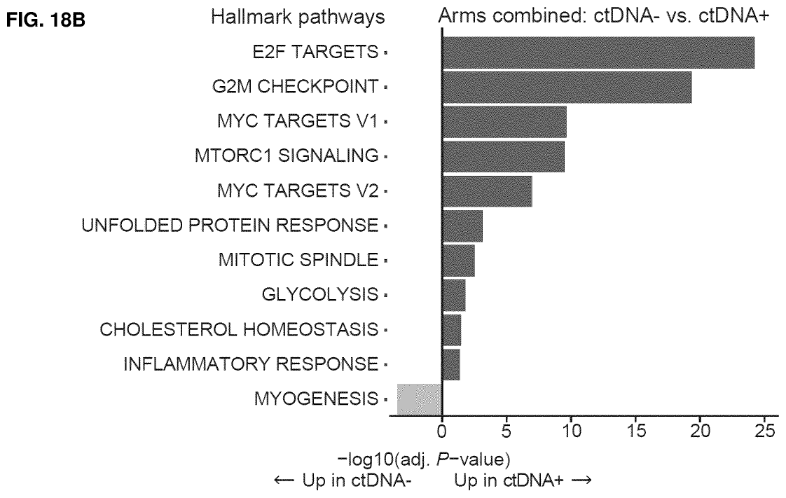

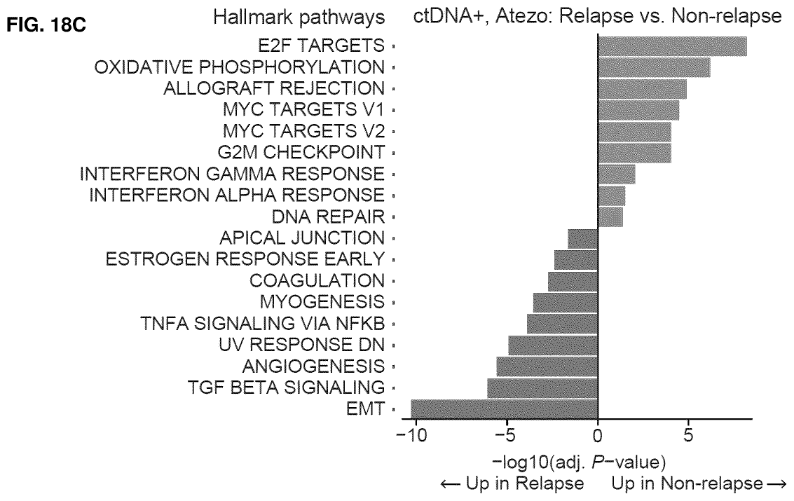

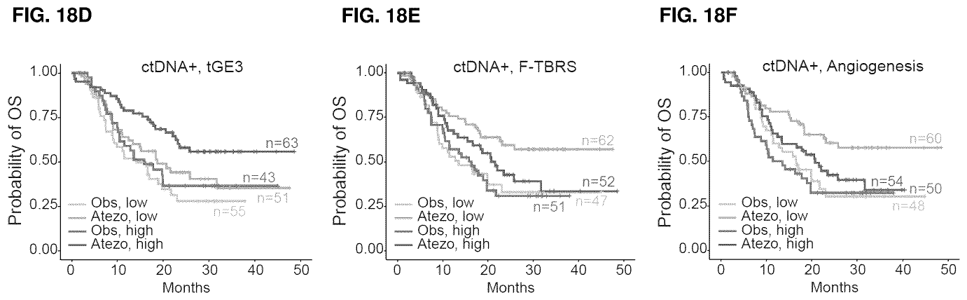

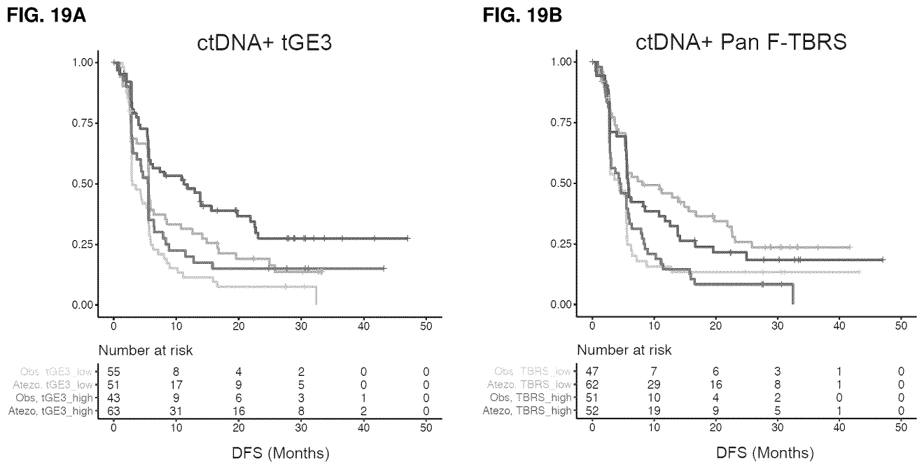

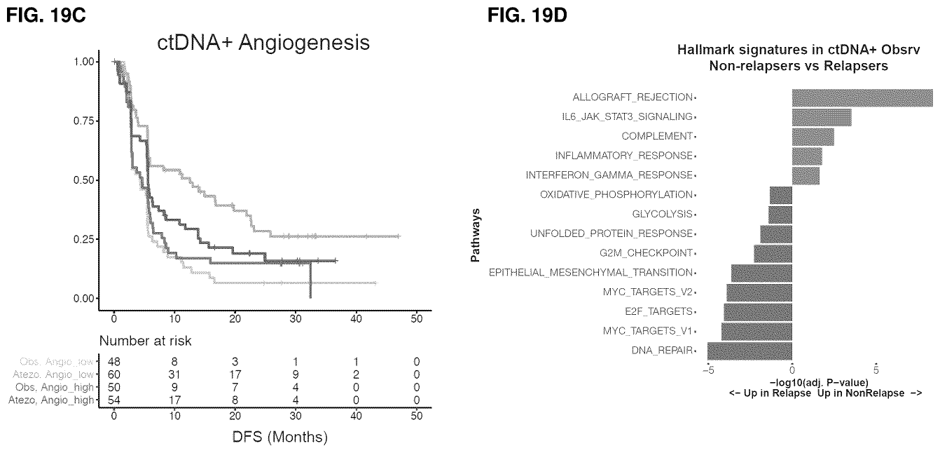

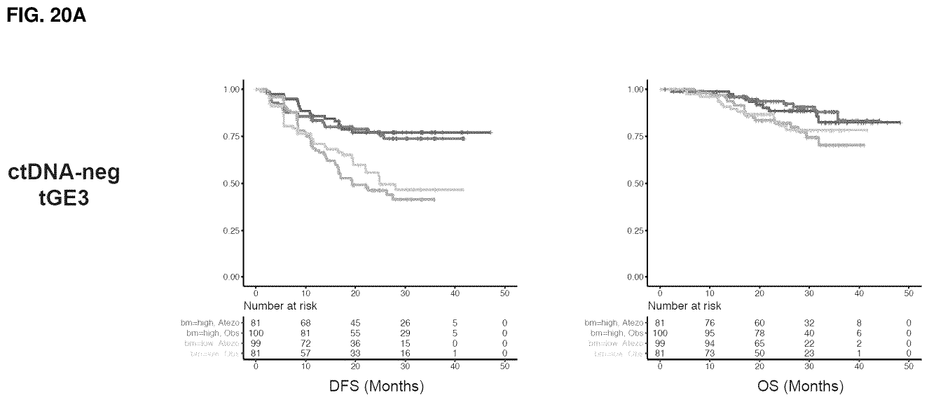

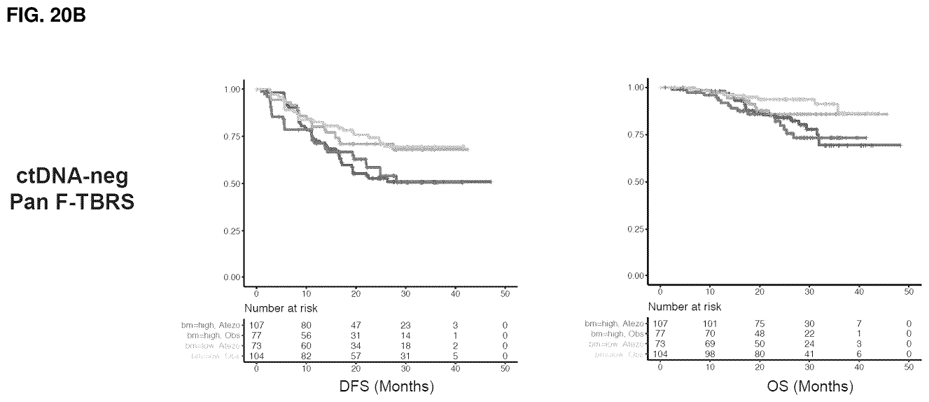

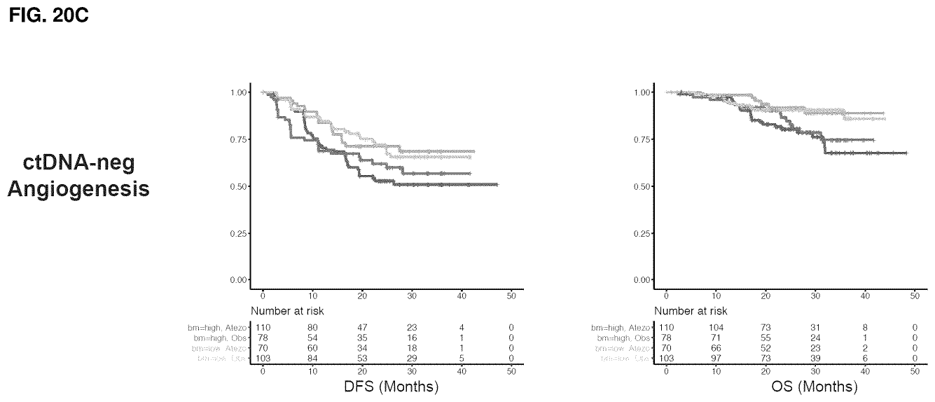

To explore the underlying mechanisms of the above findings, exploratory transcriptional analysis was performed from tumors in IMvigor010. Gene expression profiles were correlated with C1D1 ctDNA positivity and clinical relapse. Linear modelling was first applied to identify differentially expressed genes between ctDNA(+) and ctDNA(−) patients, followed by pathway enrichment analysis using the Hallmark gene sets from MSigDB (Subramanian et al. Proc Natl Acad Sci USA 102(43): 15545-15550 (2005)). Tumors from ctDNA(+) compared to ctDNA(−) patients were enriched in cell cycle and keratin genes (FIGS. 18A-18B), which may represent more aggressive cancer phenotypes. Within the ctDNA(+) patient population in the atezolizumab arm, non-relapsing patients were further enriched in interferon inducible genes, whereas relapse was associated with angiogenesis and transforming growth factor-β signaling (FIG. 18C). Next, PD-L1 and TMB were explored, which have previously been shown to select for response to immune checkpoint inhibitors across a spectrum of cancers in the metastatic setting. Their role in the adjuvant setting is uncertain. In this study, neither TMB nor PD-L1 could identify a subgroup that benefited from atezolizumab in the entire patient population (BEP). However, within the ctDNA(+) patient population, TMB(+) and PD-L1(+) enriched for improved clinical outcomes with atezolizumab (FIGS. 6A, 6B, 7A, 79, 8B, 8D, 8F, 8H, and 19A-19D), which was not observed for ctDNA negative patients (FIGS. 6A, 6B, 7A, 7B, 9A, 9B, 10A, 10B, and 20A-20C). The tGE3 (CD274, IFNG, CXCL9) signature, previously shown to identify patients who respond to atezolizumab in the metastatic setting, also enriched for improved outcomes on atezolizumab within the ctDNA(+) population (FIG. 18D). Resistance to immunotherapy in metastastic urothelial cancer is associated with high expression of the F-TBRS (pan-fibroblast TGFβ response) signature. Here we showed in the adjuvant setting that atezolizumab is also associated with worse outcomes in patients with high F-TBRS (FIG. 18E) and high angiogenesis signatures (FIG. 18F) in ctDNA(+). These data highlight that predictive biomarkers of response should be interpreted in the context of MRD in the post-surgical setting.

vii. TCGA Subtypes and Correlates of Relapse in ctDNA(−) Population

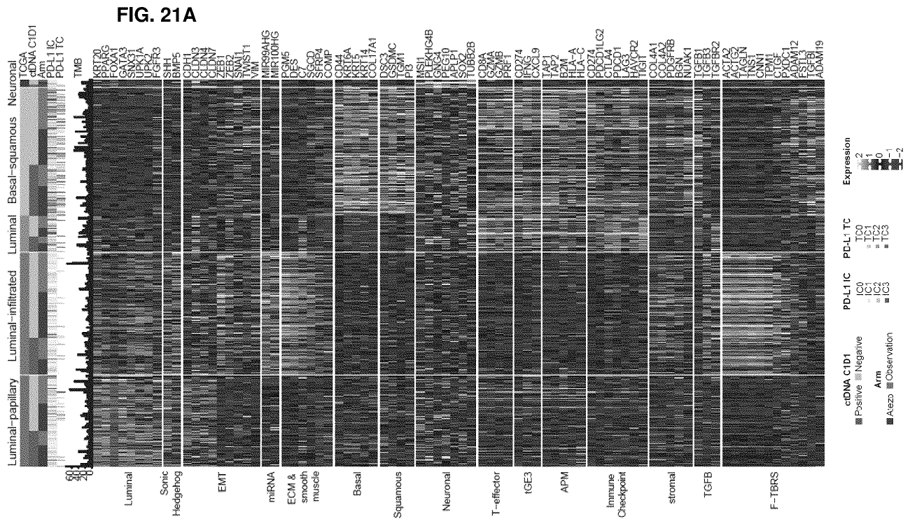

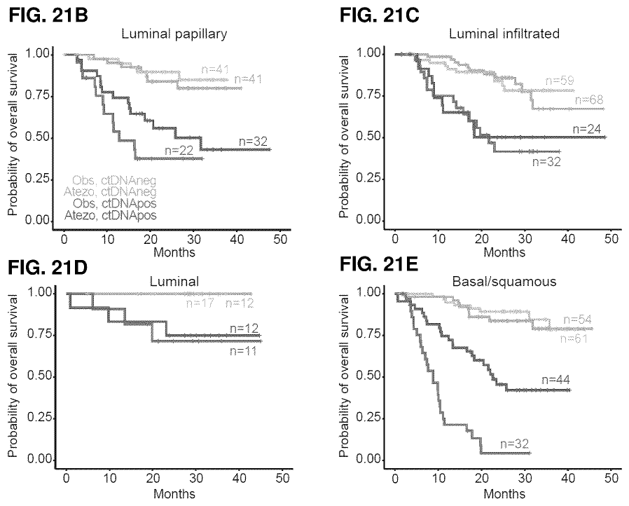

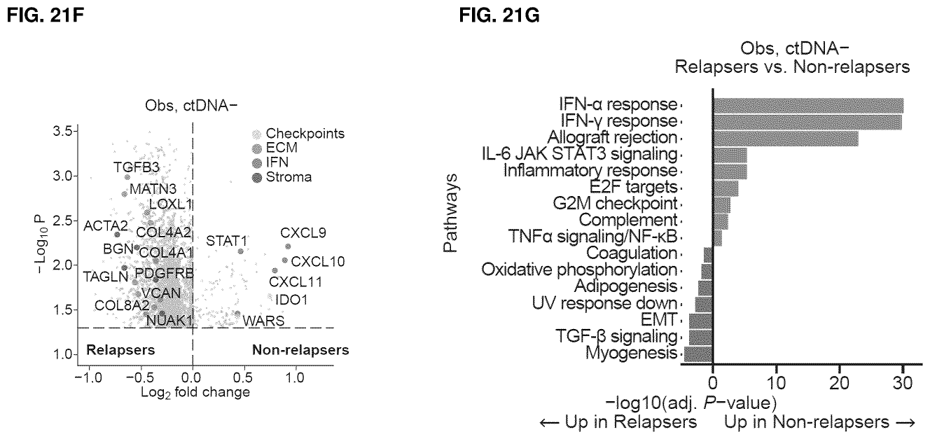

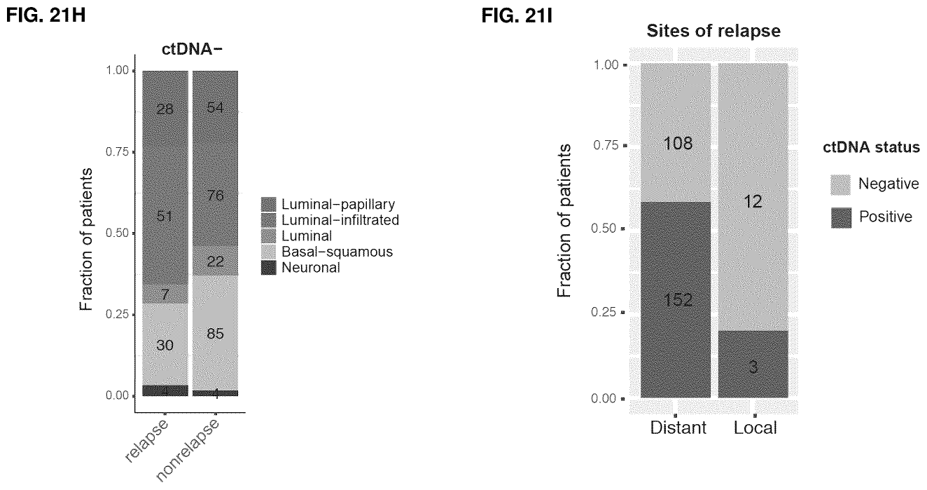

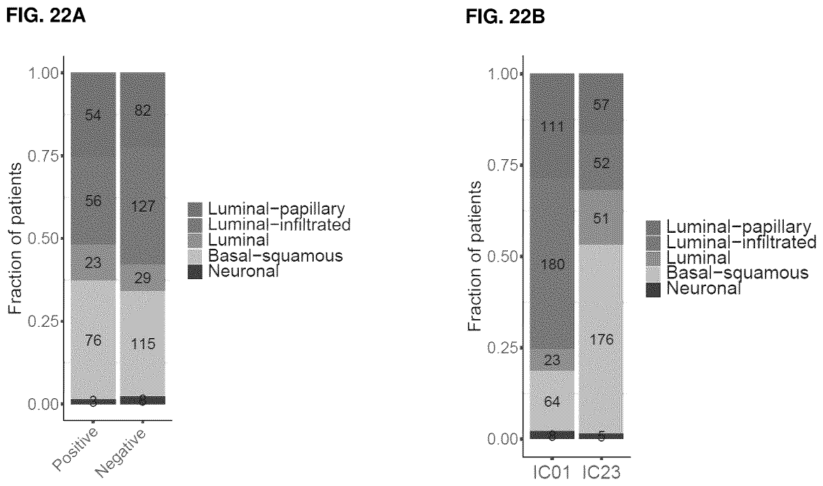

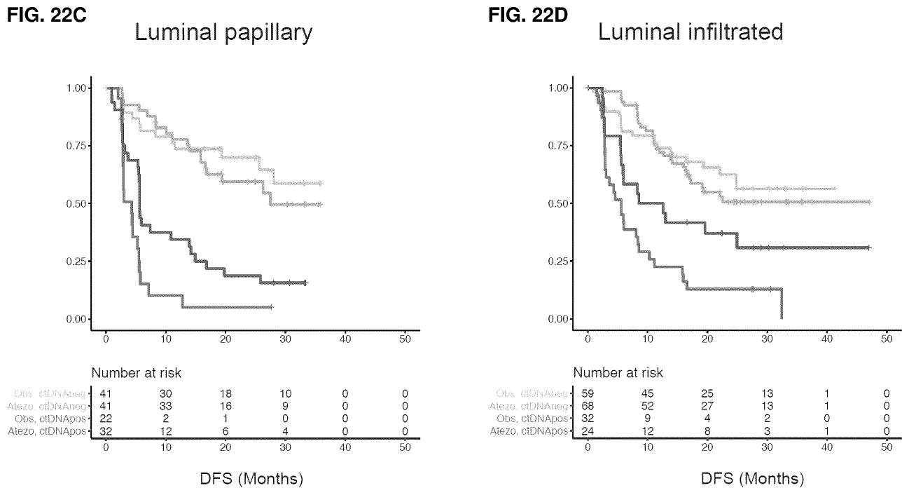

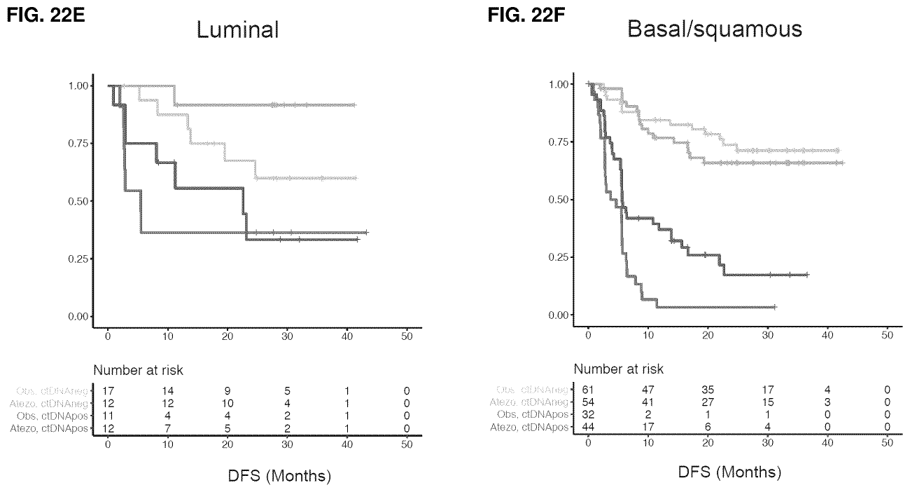

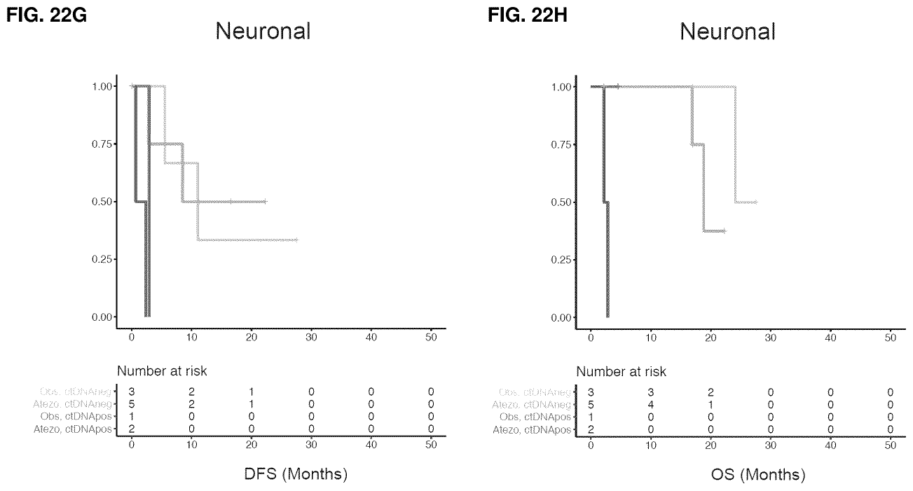

TCGA studies in urothelial cancer have identified molecular subgroups with distinct clinical characteristics (Robertson et al. Cell. 171(3): 540-556.e25 (2017)). However, it is unclear how these subtypes influence clinical outcomes from randomized data. Hierarchical clustering recapitulated the biological features in TCGA subgroups (FIG. 21A), which were distributed similarly across ctDNA(+) and ctDNA(−) patients in the BEP (FIG. 22A). In ctDNA-unselected patients, TCGA classification did not identify patient subgroups with improved outcomes with atezolizumab (FIGS. 6A, 6B, 7A, and 7B). However within the ctDNA(+) population, clinical outcomes appeared improved in the Basal-Squamous subgroup, which partially enriched for established biomarkers of response to immunotherapy (FIGS. 6A, 6B, 7A, 7B, 21B-21E, and 22A-22H) (Robertson et al. Cell 171(3): 540-556.e25 (2017)). These findings were not observed in the ctDNA(−) patients (FIGS. 6A, 6B, 7A, 7B, 9A, 9B, 10A, 10B, 20A-20C, and 21B-21E). These data suggested that TCGA analysis could be utilized to better predict outcomes of ctDNA(+) patients after surgery.

Because a subset of ctDNA(−) patients relapsed (30.6% in observation), exploratory analysis of baseline clinical parameters and molecular features of ctDNA(−) patients in the observation arm was next performed (FIGS. 21F-21I). Tumors from relapsing ctDNA(−) patients had an increase in expression of extracellular matrix (ECM), stromal, and TGFβ-inducible genes (FIG. 21F-21G), which may oppose any pre-existing immunity. The Luminal-Infiltrated TCGA subtype was also most prominent in relapsing ctDNA(−) patients (FIG. 21H). While non-relapsing ctDNA(−) patients may have had successful surgery, gene expression analysis additionally revealed increased expression of interferon (IFN) inducible genes in these patients (FIG. 21G), suggesting that pre-existing immunity may also be relevant in preventing relapse. Lastly, the anatomical location of relapse differed between ctDNA(−) and ctDNA(+) patients, where ctDNA(−) relapses were associated with local relapse and ctDNA(+) with distant relapse (FIG. 21I). These data highlight that tumor-derived molecular features may influence the relationship between ctDNA status and relapse.

viii. Discussion

This Example presents a prospective exploratory analysis of DFS and OS in patients by ctDNA for IMvigor010, a phase III trial to assess a PD-L1 inhibitor as adjuvant treatment vs. observation post-surgery in patients with high-risk for recurrence. Patients who were ctDNA(+) post-surgery were at a 6-fold increased risk of relapse and 8-fold increased risk of death compared to ctDNA(−) patients. This suggests that post-surgical ctDNA positivity may be a surrogate for MRD. Within this high-risk post-surgical ctDNA(+) population, an approximately 42% reduction in relapse rate and 41% reduction in rate of death for patients receiving atezolizumab compared to observation was found. Also, treatment with two cycles of atezolizumab led to clearance of ctDNA in 18% of ctDNA(+) patients compared to 3.8% in the observation arm. Patients who had ctDNA clearance on the atezolizumab arm had durable DFS compared to those without clearance. These findings implicate the effect of atezolizumab on outcome in ctDNA(+) patients and suggest ctDNA clearance as a possible surrogate for treatment response. No difference in clinical outcomes with atezolizumab were detected in the ctDNA(−) patients, implying that these lower risk patients (63% of the ITT) could be spared adjuvant atezolizumab treatment. These findings are clinically relevant, and the selection of a high-risk group of patients who may potentially benefit from intervention using a validated blood test is broadly attractive in the post-operative setting.

Initiating personalized treatment based on the identification of MRD rather than treating unselected patients or waiting for radiological relapse, would be a significant change in post-operative cancer treatment. This Example reveals a substantial improvement in the clinical outcomes of ctDNA(+) patients treated with adjuvant atezolizumab. These individuals are likely to have molecular residual disease after surgery. In addition, a parallel neoadjuvant atezolizumab study in UC was presented (ABACUS study), which also showed ctDNA(+) patients to have poor prognosis. In this neoadjuvant setting, reductions in ctDNA levels were associated with response, supporting the findings of the adjuvant study.

Protein and transcriptomic biomarker analysis gave insights into the biology behind ctDNA positivity and response to atezolizumab in this population, highlighting the relevance of immune and stromal contexture. The relationship between tumor-based biomarkers and ctDNA underscores that predictive biomarkers of response should be interpreted in the context of MRD, improving our understanding of the disease and response to treatment.

It has previously been shown that tissue-based TMB and PD-L1 biomarkers can be used to predict response to immune checkpoint inhibitors, especially in the metastatic setting. In IMvigor010, these tissue-based biomarkers did not identify patients who benefit from atezolizumab. However, in the ctDNA(+) population, TMB(+) or PD-L1(+) had improved outcomes compared to TMB(−) or PD-L1(−) with atezolizumab. Without wishing to be bound by theory, in the adjuvant setting, predictive biomarkers of efficacy may be most applicable to patients with MRD after surgery. A proportion of post-surgical patients will be in complete remission, and therefore tissue biomarker status will be irrelevant due to the lack of residual tumor. However, within the ctDNA(+) patients TMB and PD-L1 may provide a correlation with efficacy of checkpoint inhibition, due to the action of immunotherapy on residual tumor. PD-L1, TMB, and the Basal-Squamous transcriptomic signature was shown to potentially enrich for improved outcomes with atezolizumab in the ctDNA(+) population. A multiplatform approach may be optimal to select patients in the future. The principal of identification of a treatable post-operative population identified via a blood draw is an attractive intervention.

Numerous studies have evaluated the role of adjuvant therapies in MIUC without demonstrating a significant survival benefit. IMvigor010 was such a study; however, improvements were observed in DFS and OS in ctDNA(+) patients treated with atezolizumab compared to observation. These findings indicate that a personalized approach with immunotherapy may be optimal for the treatment of MRD(+) post-operative UC. While other adjuvant studies may be positive for DFS benefit in unselected patients, a personalized approach to select MRD(+) patients for immunotherapy may be required to demonstrate OS benefit, as well as to identify MRD(−) patients at lower risk and less likely to benefit from unnecessary treatment. Sequential testing (“surveillance” or “monitoring”) may increase sensitivity for ctDNA detection in the adjuvant setting, which is being explored in prospective trials.

In summary, this phase III trial showed that ctDNA testing after surgery can identify ctDNA(+) patients at high-risk of recurrence and death, likely due to MRD. ctDNA(+) patients had elevated rates of ctDNA clearance in the treatment arm, and improved outcomes when also positive for the TMB and PD-L1 immune biomarkers. These novel findings demonstrate ctDNA as a marker for MRD and response to atezolizumab, and link ctDNA to the biology of the tumors. Based on the totality of data, intervention with adjuvant atezolizumab can improve outcomes for select post-surgical MIUC patients, supporting atezolizumab as an important new adjuvant treatment option.

### Example 2: IMvigor011: A Phase III, Double-Blind, Multicenter, Randomized Study of Atezolizumab (Anti-PD-L1 Antibody) Versus Placebo as Adjuvant Therapy in Patients with High-Risk Muscle-Invasive Bladder Cancer Who are ctDNA-Positive Following Cystectomy

This example describes IMvigor011, a Phase III, randomized, placebo-controlled, double-blind study designed to evaluate the efficacy and safety of adjuvant treatment with atezolizumab compared with placebo in patients with MIBC who are ctDNA-positive and are at high risk for recurrence following cystectomy.

A. Objectives and Endpoints

i. Primary Efficacy Objective

The primary efficacy objective for this study is to evaluate the efficacy of atezolizumab compared with placebo on the basis of the following endpoint:
Independent Review Facility (IRF)-assessed disease-free survival (DFS) in patients who are ctDNA-positive within 20 weeks of cystectomy (primary analysis population), defined as the time from randomization to the first occurrence of a DFS event, defined as any of the following:
   Local (pelvic) recurrence of urothelial carcinoma (UC) (including soft tissue and regional lymph nodes)
   Urinary tract recurrence of UC (including all pathological stages and grades)
   Distant metastasis of UC
   Death from any cause

ii. Secondary Efficacy Objective

The secondary efficacy objective for this study is to evaluate the efficacy of atezolizumab compared with placebo on the basis of the following endpoints:
Overall survival (OS) in patients who are ctDNA-positive within 20 weeks after cystectomy (primary analysis population), defined as the time from randomization to death from any cause
IRF-assessed DFS in all randomized patients
Investigator-assessed DFS in the primary analysis population
Investigator-assessed DFS in all randomized patients
Investigator-assessed disease-specific survival in the primary analysis population, defined as the time from randomization to death from UC per investigator assessment of cause of death
Investigator-assessed distant metastasis-free survival in the primary analysis population, defined as the time from randomization to the diagnosis of distant (i.e., non-locoregional) metastases or death from any cause
Time to deterioration of function and quality of life (QoL) in the primary analysis population and in the all randomized population, defined as the time from randomization to the date of a patient's first score decrease of ≥10 points from baseline on the European Organisation for Research and Treatment of Cancer (EORTC) Quality of Life Questionnaire-Core 30 (QLQ-C30) physical function scale, role function scale, and the global health status (GHS)/QoL scale (separately)
ctDNA clearance in the primary analysis population, defined as the proportion of patients who are ctDNA-positive at baseline and ctDNA-negative at Cycle 3, Day 1 or Cycle 5, Day 1

B. Study Design

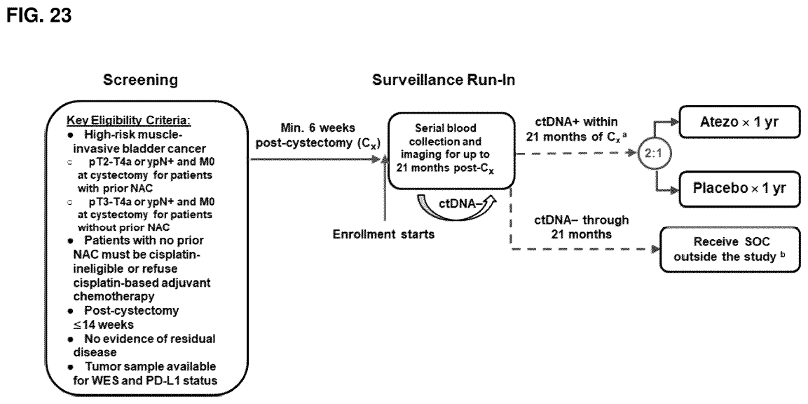

This is a global Phase III, randomized, placebo-controlled, double-blind study designed to evaluate the efficacy and safety of adjuvant treatment with atezolizumab compared with placebo in patients with MIBC who are ctDNA-positive and are at high risk for recurrence following cystectomy (see FIG. 23).

Patients aged a ≥18 years with ECOG Performance Status ≤2 who have histologically confirmed muscle-invasive urothelial carcinoma (also termed transitional cell carcinoma (TCC)) of the bladder are eligible. Patients with bladder as the site of primary involvement are required to have undergone radical cystectomy with lymph node dissection. Patients who have received prior NAC are eligible but are required to have tumor staging of ypT2-4a or ypN+ and M0 at pathological examination of the cystectomy specimen. Patients who have not received prior NAC are required to be ineligible for or declined treatment with cisplatin-based adjuvant chemotherapy and require tumor staging of pT3-4a or pN+ and M0.

Tumor tissue specimens and collection of blood from eligible patients are required for this study to prospectively test for the presence of ctDNA after surgery, to screen for eligibility into the surveillance and treatment phases, and for continued ctDNA clearance analysis or for continued ctDNA surveillance during the study. Tumor specimens from surgical resection (i.e., radical cystectomy or lymph node dissection) from patients who have provided informed consent are collected and evaluated for PD-L1 expression by immunohistochemistry (IHC). Tumor specimens also undergo whole exome sequencing (WES). Blood samples are collected to determine both normal DNA and ctDNA in the patient's blood. Only patients whose tumors have sufficient amounts of viable tumor for WES and are evaluable for PD-L1 expression, as confirmed by a central pathology laboratory prior to enrollment of the patient in the study, are eligible. Patients' tumor specimens are sequenced against matched normal DNA to create a panel of multiplex polymerase chain reaction (mPCR) assays for the top 16 clonal mutations unique to each patient's tumor tissue.

All eligible patients with personalized mPCR assays, regardless of plasma ctDNA status, are enrolled in the surveillance phase of the study, provided that they have consented to participate in the surveillance phase and have no residual disease as assessed by IRF. Patients may be enrolled in the surveillance phase a minimum of 6 weeks but not more than 14 weeks from the date of cystectomy.

Patients enrolled in the surveillance phase undergo blood collection for plasma ctDNA testing and surveillance imaging for tumor recurrence. Blood collection occurs every 6 weeks from the date of enrollment until Week 36 or until 36 weeks from the date of cystectomy have passed, whichever occurs first. After the latest blood collection prior to 36 weeks from cystectomy has been reached, blood collection follows the surveillance imaging schedule going forward. Surveillance imaging for the surveillance phase is performed every 12 weeks from the date of enrollment until Week 84 or until 21 months from the date of cystectomy have passed, whichever occurs first. Patients are discontinued from the surveillance phase in the event of investigator-assessed disease recurrence.

Patients' blood samples collected during the surveillance phase are evaluated for the presence of up to 16 mutations identified from the primary tumor. Plasma samples evaluated to have 2 or more mutations are considered ctDNA-positive. Patients enter the treatment phase of the study and are randomized to treatment at the first plasma sample that is ctDNA-positive provided that they have fully recovered from cystectomy, provided that there is no evidence of disease recurrence on imaging within 28 days prior to treatment initiation as per IRF assessment, and provided that they have consented to participate in the treatment phase. Only patients who are ctDNA-positive will enter the treatment phase. Patients who are ctDNA-negative will continue to undergo surveillance until they are either ctDNA-positive, ctDNA-negative at 21 months from the date of their cystectomy, or have investigator-assessed radiographic recurrence.

Tumor tissue specimens from patients are also prospectively tested for PD-L1 expression by a central laboratory during the screening period, and PD-L1 status (IHC score of IC0/1 vs. IC2/3) is used as one of the stratification factors.

Patients entering the treatment phase are randomized to one of the following arms in a 2:1 ratio:
Arm A (experimental arm): atezolizumab 1680 mg IV infusion every 4 weeks (Q4W) on Day 1 of each 28-day cycle
Arm B (control arm): placebo IV infusion 04W on Day 1 of each 28-day cycle

Patients in both treatment arms will receive 12 cycles or up to 1 year (whichever occurs first) of treatment with either atezolizumab (fixed dose of 1680 mg) or matching placebo. Treatment will be administered by IV infusion on Day 1 of each 28-day cycle.

Atezolizumab/placebo are discontinued in the event of IRF-assessed disease recurrence, unacceptable toxicity, withdrawal of consent, or study termination.

Randomization is stratified by the following factors:
Nodal status (positive vs. negative)
Tumor stage after cystectomy (≤pT2 vs. pT3/pT4)
PD-L1 IHC status (IHC score of IC0/1 vs. IC2/3)
   PD-L1 expression (IC2/3, corresponding to the presence of discernible PD-L1 staining of any intensity in tumor-infiltrating immune cells covering a ≥5% of tumor area occupied by tumor cells, associated intratumoral, and contiguous peritumoral stroma) is assessed by a central laboratory using the VENTANA PD-L1 (SP142) Assay.
Time from cystectomy to first ctDNA-positive sample (≤20 weeks vs.>20 weeks)

Randomization occurs within 14 days of a patients' plasma sample being confirmed as ctDNA-positive. Study drug administration begins within 4 calendar days of randomization.

All patients entered in the treatment phase undergo scheduled assessments for tumor recurrence at baseline and every 9 weeks (±7 days) in the first year following randomization. Upon completion of the treatment/placebo phase, disease status assessments for tumor recurrence are performed every 9 weeks (±7 days) for Year 2; every 12 weeks (±10 days) for Year 3; every 24 weeks (±10 days) for Years 4-5; and at Year 6 (approximately 48 weeks after the last assessment in Year 5).

Patients who remain ctDNA-negative at 21 months from the date of cystectomy are not randomized to treatment and are discontinued from the study.

C. Materials and Methods

i. Inclusion Criteria

Patients are required to meet the following criteria for study entry:

Inclusion Criteria for the Surveillance Phase
Histologically confirmed MIUC (also termed TCC) of the bladder
   Patients with carcinomas showing mixed histologies are required to have a dominant transitional cell pattern
TNM classification (based on AJCC Cancer Staging Manual, 7th Edition; Edge et al. 2010) at pathological examination of surgical resection specimen as follows:
   For patients treated with prior NAC: tumor stage of ypT2-4a or ypN+ and M0
   For patients who have not received prior NAC: tumor stage of pT3-4a or pN+ and M0
Surgical resection of MIUC of the bladder
   Radical cystectomy may be performed by the open, laparoscopic, or robotic approach. Cystectomy is required to include bilateral lymph node dissection, the extent of which is at the discretion of the treating surgeon but optimally should extend at a minimum from the mid common iliac artery proximally to Cooper's ligament distally, laterally to the genitofemoral nerve, and inferiorly to the obturator nerve. The method of urinary diversion for patients undergoing cystectomy is at the discretion of the surgeon and choice of the patient.
   Patients with a negative surgical margin (i.e., R0 resection) or with carcinoma in situ at the distal ureteral or urethral margin are eligible.
   Patients with a positive R2 margin (which is defined as a tumor identified at the inked perivesical fat margin surrounding the cystectomy specimen) or R1 margin (which is defined as evidence of microscopic disease identified at the tumor margin), except for carcinoma in situ at the distal ureteral or urethral margin, are excluded.
Patients who have not received prior platinum-based NAC, have refused, or are ineligible (“unfit”) for cisplatin-based adjuvant chemotherapy
   Patients who have received at least three cycles of a platinum-containing regimen are considered as having received prior NAC.
   Cisplatin ineligibility is defined by any one of the following criteria:
      Impaired renal function (glomerular filtration rate (GFR)<60 mL/min); GFR should be assessed by direct measurement (i.e., creatinine clearance or ethyldediaminetetra-acetate) or, if not available, by calculation from serum/plasma creatinine (Cockcroft Gault formula)
      A hearing loss (measured by audiometry) of 25 dB at two contiguous frequencies
      Grade 2 or greater peripheral neuropathy (i.e., sensory alteration or parasthesis including tingling)
      ECOG Performance Status of 2
Availability of a surgical tumor specimen that is suitable (e.g., adequate quality and quantity) for use in determining ctDNA status and for exploratory biomarker research assessed by central laboratory testing. Representative formalin-fixed, paraffin-embedded (FFPE) tumor block submitted along with an associated pathology report; two FFPE tumor blocks are recommended, if available. Patients with fewer than 20 slides available at baseline (but no fewer than 16) may still be eligible for the study after Medical Monitor approval has been obtained.
Tumor tissue specimen submitted within 10 weeks of cystectomy for ctDNA assay development.
ctDNA assay developed based on tumor tissue specimen and matched normal DNA from blood.
A post-surgery blood sample submitted for screening for the identification of somatic mutations in tumor tissue and for plasma preparation for determining ctDNA status
Tumor PD-L1 expression per IHC and confirmed diagnosis of MIUC as documented through central testing of a representative tumor tissue specimen
Absence of residual disease and absence of metastasis, as confirmed by a negative baseline computed tomography (CT) or magnetic resonance imaging (MRI) scan of the pelvis, abdomen, and chest no more than 4 weeks prior to enrollment.
   Confirmation of disease-free status is assessed by an independent central radiologic review of imaging data
   Imaging of the upper urinary tracts is required and may include one or more of the following: intravenous pyelogram (IVP), CT urography, renal ultrasound with retrograde pyelogram, ureteroscopy or MRI urogram. However, separate imaging of the upper urinary tracts via one of these modalities is not required if the upper tracts are covered in the imaging of the abdomen and pelvis. Imaging must be completed no more than 4 weeks prior to enrollment
Full recovery from cystectomy and enrollment within 14 weeks following cystectomy
   Minimum of 6 weeks must have elapsed from surgery

Additional Inclusion Criteria for the Treatment Phase

Patients enrolled in the surveillance phase are required to meet the following criteria for randomization into the treatment phase of the study:
Plasma sample evaluated to be ctDNA-positive, defined as the presence of two or more mutations based on patient's personalized ctDNA mPCR assay.
ECOG Performance Status of ≤2
Absence of residual disease and absence of metastasis, as confirmed by a negative baseline CT or MRI scan of the pelvis, abdomen, and chest no more than 4 weeks prior to randomization.
   Confirmation of disease-free status is assessed by an independent central radiologic review of imaging data.
   Imaging of the upper urinary tracts is required and may include one or more of the following: IVP, CT urography, renal ultrasound with retrograde pyelogram, ureteroscopy or MRI urogram. However, separate imaging of the upper urinary tracts via one of these modalities is not required if the upper tracts are covered in the imaging of the abdomen and pelvis. Imaging must be completed no more than 4 weeks prior to enrollment.

ii. Exclusion Criteria

Patients who meet any of the following criteria are excluded from study entry:
History of severe allergic, anaphylactic, or other hypersensitivity reactions to chimeric or humanized antibodies or fusion proteins
Known hypersensitivity to biopharmaceuticals produced in Chinese hamster ovary cells or any component of the atezolizumab formulation
Any approved anti-cancer therapy, including chemotherapy, or hormonal therapy within 3 weeks prior to study enrollment.
   Hormone-replacement therapy or oral contraceptives are allowed.
Adjuvant chemotherapy or radiation therapy for UC following cystectomy
   Patients who have received primary chemoradiation for bladder preservation before cystectomy are eligible and will be treated as the same as patients who have received prior NAC.

Patients in the surveillance phase who meet any of the following additional medication-related criteria are excluded from entry in the treatment phase:
Prior treatment with CD137 agonists or immune checkpoint-blockade therapies, including anti-CD40, anti-CTLA-4, anti-PD-1, and anti-PD-L1 therapeutic antibodies
Treatment with systemic immunostimulatory agents (including, but not limited to, IFNs, IL-2) within 6 weeks or 5 half-lives of the drug, whichever is shorter, prior to Cycle 1, Day 1

iii. Study Treatment

The investigational medicinal product (IMP) for this study is atezolizumab. The placebo will be identical in appearance to atezolizumab and will comprise the same excipients but without atezolizumab Drug Product. Atezolizumab/placebo will be administered by IV infusion at a fixed dose of 1680 mg on Day 1 of each 28-day (±3 days) cycle for 12 cycles or 1 year, whichever occurs first. This dose level is equivalent to an average body weight-based dose of approximately 20 mg/kg.

iv. Statistical Analysis

The primary efficacy endpoint is IRF-assessed DFS, defined as the time from randomization to the first occurrence of a DFS event. DFS is analyzed in the primary analysis population, defined as randomized patients with a ctDNA-positive sample obtained within 20 weeks following cystectomy. Data for patients without a DFS event are censored at the last date the patient was assessed to be alive and recurrence free. Data for patients with no post-baseline disease assessment are censored at the randomization date.

DFS is compared between treatment arms using the stratified log-rank test. The null and alternative hypotheses can be phrased in terms of the survival functions S_{A} (t) and S_{B} (t) in Arm A (atezolizumab) and Arm B (placebo), respectively:

H₀:S_{A}(t)=S_{B}(t)versus H₁:S_{A}(t)≠S_{B}(t)

The HR, λ_{A}/λ_{B}, where λ_{A} and λ_{B} represent the hazard of a DFS event in Arm A and Arm B respectively, will be estimated using a stratified Cox regression model with the same stratification variables used for the stratified log-rank test, and the 95% CI is provided. Results from an unstratified analysis will also be provided. HR<1 indicates treatment benefit in favor of atezolizumab. The stratification factors for the primary analysis population will include nodal status, tumor stage after cystectomy, PD-L1 IHC status, and time from cystectomy to first ctDNA-positive sample; however, stratification factors may be combined for analysis purposes if necessary to minimize small stratum cell sizes.

The type 1 error (α) for this study is 0.05 (two-sided). Type 1 error is controlled for the primary endpoint of IRF-assessed DFS and the key secondary endpoint of OS for the primary analysis population and for IRF-assessed DFS for the all randomized population. To control the type 1 error at α=0.05 (two-sided) for the IRF-assessed DFS and OS endpoints, the treatment arms are compared in a hierarchical fashion as follows: Step 1: IRF-assessed DFS for the primary analysis population is evaluated at α=0.05 (two-sided). Step 2: If the IRF-assessed DFS analysis results for the primary analysis population are statistically significant, α=0.05 is passed to the analysis of OS for the primary analysis population. If the IRF-assessed DFS results for the primary analysis population are not statistically significant, formal treatment comparison of OS is not performed. Step 3: If the OS results in the primary analysis population are statistically significant at either interim or the final OS analysis, α=0.05 is passed to the analysis of IRF-assessed DFS in the all randomized population. If the OS for primary analysis population results are not statistically significant at either interim or the final analysis, formal treatment comparison of IRF-assessed DFS in the all randomized population is not be performed.

Kaplan-Meier methodology is used to estimate median DFS for each treatment arm; Kaplan-Meier curves are produced. Brookmeyer-Crowley methodology is used to construct the 95% CI for the median DFS for each treatment arm. The DFS rate at various timepoints (i.e., every 6 months after randomization) is estimated by Kaplan-Meier methodology for each treatment arm, and the 95% CI is calculated using Greenwood's formula. The 95% CI for the difference in rates between the two arms is estimated using the normal approximation method.

Additional analyses are performed for both IRF-assessed DFS endpoints described above, including analyses at selected timepoints and subgroup analyses.

IRF-assessed DFS is formally analyzed in the all randomized population (i.e., all patients randomized to treatment regardless of the length of time between cystectomy and ctDNA-positive status) if both the IRF-assessed DFS and OS analysis results for the primary analysis population are statistically significant. In that circumstance, a nominal amount of a (i.e., 0.0001) is allocated to each OS interim analysis to maintain familywise Type I error control for IRF-assessed DFS in the all randomized population (Haybittle-Peto boundary). This approach for Type I error control accounts for unblinding study results prior to the analysis of IRF-assessed DFS in the all randomized population, as the primary analysis population is included in the analysis of the all randomized population.

A secondary efficacy endpoint is OS, defined as the time from randomization to death from any cause. OS is analyzed in the primary analysis population, defined as randomized patients with a ctDNA-positive sample obtained within 20 weeks following cystectomy. Methods for comparison of OS between treatment arms are the same as the methods for treatment comparison for the primary efficacy endpoint of IRF-assessed DFS. The boundaries for statistical significance at the interim and final OS analyses will be determined based on the Lan-DeMets implementation of the O'Brien-Fleming use function. OS is also analyzed in the all randomized population as an exploratory analysis using the same methodology as for OS in the primary analysis population.

ctDNA clearance, defined as the proportion of patients ctDNA-positive at baseline and ctDNA-negative at Cycle 3, Day 1 or Cycle 5, Day 1, is analyzed in the primary analysis population. An estimate of the proportion of patients with ctDNA clearance and its 95% CI is calculated using the Clopper-Pearson method for each treatment arm. The CI for the difference in the proportion between the two arms is determined using the normal approximation to the binomial distribution. The proportions are compared between the two arms with the use of the stratified Cochran-Mantel-Haenszel test.

### Other Embodiments

Some embodiments of the technology described herein can be defined according to any of the following numbered embodiments:

1. A method of treating urothelial carcinoma in a patient in need thereof, the method comprising administering to the patient an effective amount of a treatment regimen comprising a PD-1 axis binding antagonist, wherein the treatment regimen is an adjuvant therapy, and wherein the patient has been identified as likely to benefit from the treatment regimen based on the presence of circulating tumor DNA (ctDNA) in a biological sample obtained from the patient.

2. A method of treating urothelial carcinoma in a patient in need thereof, the method comprising:
(a) determining whether ctDNA is present in a biological sample obtained from the patient, wherein the presence of ctDNA in the biological sample indicates that the patient is likely to benefit from a treatment regimen comprising a PD-1 axis binding antagonist; and
(b) administering an effective amount of a treatment regimen comprising a PD-1 axis binding antagonist to the patient based on the presence of ctDNA in the biological sample, wherein the treatment regimen is an adjuvant therapy.

3. A method of identifying a patient having a urothelial carcinoma who may benefit from a treatment regimen comprising a PD-1 axis binding antagonist, the method comprising determining whether ctDNA is present in a biological sample obtained from the patient, wherein the presence of ctDNA in the biological sample identifies the patient as one who may benefit from treatment with a treatment regimen comprising a PD-1 axis binding antagonist, wherein the treatment regimen is an adjuvant therapy.

4. A method for selecting a therapy for a patient having a urothelial carcinoma, the method comprising
(a) determining whether ctDNA is present in a biological sample obtained from the patient, wherein the presence of ctDNA in the biological sample indicates that the patient is likely to benefit from a treatment regimen comprising a PD-1 axis binding antagonist; and
(b) selecting a treatment regimen comprising a PD-1 axis binding antagonist based on the presence of ctDNA in the biological sample, wherein the treatment regimen is an adjuvant therapy.

5. The method of embodiment 3 or 4, further comprising administering an effective amount of a treatment regimen comprising a PD-1 axis binding antagonist to the patient.

6. The method of any one of embodiments 1-5, wherein the biological sample is obtained prior to or concurrently with administration of a first dose of the treatment regimen.

7. The method of embodiment 6, wherein the biological sample is obtained on cycle 1, day 1 (C1D1) of the treatment regimen.

8. The method of any one of embodiments 1-7, wherein the biological sample is obtained within about 30 weeks from surgical resection.

9. The method of embodiment 8, wherein the biological sample is obtained within about 20 weeks from surgical resection.

10. The method of embodiment 8 or 9, wherein the biological sample is obtained about 2 to about 20 weeks after surgical resection.

11. The method of any one of embodiments 1-10, wherein the biological sample is a blood sample, a plasma sample, a serum sample, a urine sample, a cerebrospinal fluid (CSF) sample, a nasal swab sample, a saliva sample, a stool sample, or a vaginal fluid sample.

12. The method of embodiment 11, wherein the biological sample is a plasma sample.

13. A method of monitoring the response of a patient having a urothelial carcinoma who has been administered at least a first dose of a treatment regimen comprising a PD-1 axis binding antagonist, wherein the treatment regimen is a neoadjuvant therapy or an adjuvant therapy, and wherein ctDNA was present in a biological sample obtained from the patient prior to or concurrently with the first dose of the treatment regimen, the method comprising determining whether ctDNA is present in a biological sample obtained from the patient at a time point following administration of the first dose of the treatment regimen, thereby monitoring the response of the patient.

14. The method of embodiment 13, wherein an absence of ctDNA in the biological sample obtained from the patient at a time point following administration of the first dose of the treatment regimen indicates that the patient is responding to the treatment regimen.

15. A method of identifying a patient having a urothelial carcinoma who may benefit from a treatment regimen comprising a PD-1 axis binding antagonist, wherein the treatment regimen is a neoadjuvant therapy or an adjuvant therapy and the patient has been administered at least a first dose of the treatment regimen, and wherein ctDNA was present in a biological sample obtained from the patient prior to or concurrently with the first dose of the treatment regimen, the method comprising: determining whether ctDNA is present in a biological sample obtained from the patient at a time point following administration of the first dose of the treatment regimen, wherein an absence of ctDNA in the biological sample at the time point following administration of the treatment regimen identifies the patient as one who may benefit from treatment with a treatment regimen comprising a PD-1 axis binding antagonist.

16. The method of any one of embodiments 13-15, wherein the treatment regimen is an adjuvant therapy.

17. The method of any one of embodiments 13-16, wherein the time point following administration of the first dose of the treatment regimen is on cycle 3, day 1 (C3D1) or cycle 5, day 1 (C5D1) of the treatment regimen.

18. The method of any one of embodiments 13-17, wherein the biological sample obtained from the patient prior to or concurrently with a first dose of the treatment regimen and/or the biological sample obtained from the patient at a time point following administration of the first dose of the treatment regimen is a blood sample, a plasma sample, a serum sample, a urine sample, a CSF sample, a nasal swab sample, a saliva sample, a stool sample, or a vaginal fluid sample.

19. The method of embodiment 18, wherein the biological sample obtained from the patient prior to or concurrently with a first dose of the treatment regimen and/or the biological sample obtained from the patient at a time point following administration of the first dose of the treatment regimen is a plasma sample.

20. The method of any one of embodiments 1-12 and 15-19, wherein the benefit is in terms of improved disease-free survival (DFS), improved overall survival (OS), improved disease-specific survival, or improved distant metastasis-free survival.

21. The method of embodiment 20, wherein the benefit is in terms of improved DFS.

22. The method of embodiment 20, wherein the benefit is in terms of improved OS.

23. The method of any one of embodiments 20-22, wherein improvement is relative to observation or relative to adjuvant therapy with a placebo.

24. The method of any one of embodiments 1-23, wherein the presence of ctDNA is determined by a polymerase chain reaction (PCR)-based approach, a hybridization capture-based approach, a methylation-based approach, or a fragmentomics approach.

25. The method of embodiment 24, wherein the presence of ctDNA is determined by a personalized ctDNA multiplexed polymerase chain reaction (mPCR) approach.

26. The method of embodiment 25, wherein the personalized ctDNA mPCR approach comprises:
(a)
(i) sequencing DNA obtained from a tumor sample obtained from the patient to produce tumor sequence reads; and
(ii) sequencing DNA obtained from a normal tissue sample obtained from the patient to produce normal sequence reads;
(b) identifying one or more patient-specific variants by calling somatic variants identified from the tumor sequence reads and excluding germline variants or clonal hematopoiesis of indeterminate potential (CHIP) variants, wherein the germline variants or CHIP variants are identified from the normal sequence reads or from a publicly available database;
(c) designing an mPCR assay for the patient that detects a set of patient-specific variants; and
(d) analyzing a biological sample obtained from the patient using the mPCR assay to determine whether ctDNA is present in the biological sample.

27. The method of embodiment 26, wherein the sequencing is whole-exome sequencing (WES) or whole-genome sequencing (WGS).

28. The method of embodiment 27, wherein the sequencing is WES.

29. The method of any one of embodiments 26-28, wherein the patient-specific variants are single nucleotide variants (SNVs) or short indels.

30. The method of any one of embodiments 26-29, wherein the set of patient-specific variants comprises at least 2 patient-specific variants.

31. The method of embodiment 30, wherein the set of patient-specific variants comprises 2 to 200 patient-specific variants.

32. The method of embodiment 31, wherein the set of patient-specific variants comprises 16 patient-specific variants.

33. The method of any one of embodiments 26-32, wherein analyzing the biological sample obtained from the patient using the mPCR assay comprises sequencing amplicons produced by the mPCR assay to identify patient-specific variants in the biological sample.

34. The method of any one of embodiments 25-33, wherein the personalized ctDNA mPCR approach is a SIGNATERA® ctDNA test or an ArcherDx Personalized Cancer Monitoring (PCM™) test.

35. The method of any one of embodiments 25-34, wherein the presence of at least one patient-specific variant in the biological sample identifies the presence of ctDNA in the biological sample.

36. The method of embodiment 35, wherein the presence of two patient-specific variants in the biological sample identifies the presence of ctDNA in the biological sample.

37. The method of any one of embodiments 1-36, wherein the urothelial carcinoma is muscle-invasive urothelial carcinoma (MIUC).

38. The method of embodiment 37, wherein the MIUC is muscle-invasive bladder cancer (MIBC) or muscle-invasive urinary tract urothelial cancer (muscle-invasive UTUC).

39. The method of embodiment 37 or 38, wherein the MIUC is histologically confirmed and/or wherein the patient has an Eastern Cooperative Oncology Group (ECOG) Performance Status of less than or equal to 2.

40. The method of any one of embodiments 37-39, wherein the patient has previously been treated with neoadjuvant chemotherapy.

41. The method of embodiment 40, wherein the patient's MIUC is ypT2-4a or ypN+ and M0 at surgical resection.

42. The method of any one of embodiments 37-41, wherein the patient has not received prior neoadjuvant chemotherapy.

43. The method of embodiment 42, wherein the patient is cisplatin-ineligible or has refused cisplatin-based adjuvant chemotherapy.

44. The method of embodiment 42 or 43, wherein the patient's MIUC is pT3-4a or pN+ and M0 at surgical resection.

45. The method of any one of embodiments 1-44, wherein the patient has undergone surgical resection with lymph node dissection.

46. The method of embodiment 45, wherein the surgical resection is cystectomy or nephroureterectomy.

47. The method of any one of embodiments 1-46, wherein the patient has no evidence of residual disease or metastasis as assessed by postoperative radiologic imaging.

48. The method of any one of embodiments 1-47, wherein a tumor sample obtained from the patient has been determined to have a detectable expression level of PD-L1 in tumor-infiltrating immune cells that comprise about 1% or more of the tumor sample.

49. The method of embodiment 48, wherein the tumor sample has been determined to have a detectable expression level of PD-L1 in tumor-infiltrating immune cells that comprise about 1% or more to less than 5% of the tumor sample.

50. The method of embodiment 48, wherein the tumor sample has been determined to have a detectable expression level of PD-L1 in tumor-infiltrating immune cells that comprise about 5% or more of the tumor sample.

51. The method of embodiment 50, wherein the tumor sample has been determined to have a detectable expression level of PD-L1 in tumor-infiltrating immune cells that comprise about 5% or more to less than 10% of the tumor sample.

52. The method of embodiment 48 or 50, wherein the tumor sample obtained from the patient has been determined to have a detectable expression level of PD-L1 in tumor-infiltrating immune cells that comprise about 10% or more of the tumor sample.

53. The method of any one of embodiments 1-47, wherein a tumor sample obtained from the patient has been determined to have a detectable expression level of PD-L1 in tumor-infiltrating immune cells that comprise less than 1% of the tumor sample.

54. The method of any one of embodiments 1-53, wherein a tumor sample obtained from the patient has been determined to have a tissue tumor mutational burden (tTMB) score that is at or above a reference tTMB score.

55. The method of embodiment 54, wherein the reference tTMB score is a pre-assigned tTMB score.

56. The method of embodiment 55, wherein the pre-assigned tTMB score is between about 8 and about 30 mut/Mb.

57. The method of embodiment 56, wherein the pre-assigned tTMB score is about 10 mutations per megabase (mut/Mb).

58. The method of any one of embodiments 48-57, wherein the tumor sample is from surgical resection.

59. The method of any one of embodiments 1-58, wherein the patient has an increased expression level of one or more genes selected from PD-L1, IFNG, and CXCL9 relative to a reference expression level of the one or more genes in a biological sample obtained from the patient.

60. The method of embodiment 59, wherein the patient has an increased expression level of two or more genes selected from PD-L1, IFNG, and CXCL9 relative to a reference expression level of the two or more genes in the biological sample obtained from the patient.

61. The method of embodiment 60, wherein the patient has an increased expression level of PD-L1, IFNG, and CXCL9 relative to a reference expression level of PD-L1, IFNG, and CXCL9 in the biological sample obtained from the patient.

62. The method of any one of embodiments 59-61, wherein the expression level of PD-L1, IFNG, and/or CXCL9 is an mRNA expression level.

63. The method of any one of embodiments 1-62, wherein the patient has a decreased expression level of one or more pan-F-TBRS genes selected from ACTA2, ACTG2, TAGLN, TNS1, CNN1, TPM1, CTGF, PXDC1, ADAM12, FSTL3, TGFBI, and ADAM19 relative to a reference expression level of the one or more pan-F-TBRS genes in a biological sample obtained from the patient.

64. The method of embodiment 63, wherein the patient has a decreased expression level of at least two, at least three, at least four, at least five, at least six, at least seven, at least eight, at least nine, at least ten, at least eleven, or all twelve of the pan-F-TBRS genes relative to a reference expression level of the at least two, at least three, at least four, at least five, at least six, at least seven, at least eight, at least nine, at least ten, at least eleven, or all twelve pan-F-TBRS genes in the biological sample obtained from the patient.

65. The method of embodiment 63 or 64, wherein the expression level of the one or more pan-F-TBRS genes is an mRNA expression level.

66. The method of any one of embodiments 59-65, wherein the biological sample obtained from the patient is a tumor sample.

67. The method of any one of embodiments 1-66, wherein the patient's tumor has a basal-squamous subtype.

68. The method of embodiment 67, wherein the patient has an increased expression level of one or more genes selected from CD44, KRT6A, KRT5, KRT14, COL17A1, DSC3, GSDMC, TGM1, and PI3 relative to a reference expression level of the one or more genes.

69. The method of any one of embodiments 1-68, wherein the PD-1 axis binding antagonist is selected from a PD-L1 binding antagonist, a PD-1 binding antagonist, and a PD-L2 binding antagonist.

70. The method of embodiment 69, wherein the PD-1 axis binding antagonist is a PD-L1 binding antagonist.

71. The method of embodiment 70, wherein the PD-L1 binding antagonist is an anti-PD-L1 antibody.

72. The method of embodiment 71, wherein the anti-PD-L1 antibody is atezolizumab, durvalumab, avelumab, or MDX-1105.

73. The method of embodiment 72, wherein the anti-PD-L1 antibody is atezolizumab.

74. The method of embodiment 73, wherein the atezolizumab is administered intravenously every two weeks at a dose of 840 mg.

75. The method of embodiment 73, wherein the atezolizumab is administered intravenously every three weeks at a dose of 1200 mg.

76. The method of embodiment 73, wherein the atezolizumab is administered intravenously every four weeks at a dose of 1680 mg.

77. The method of embodiment 76, wherein the atezolizumab is administered on Day 1 of each 28-day (±3 days) cycle for 12 cycles or one year, whichever occurs first.

78. The method of embodiment 69, wherein the PD-1 axis binding antagonist is a PD-1 binding antagonist

79. The method of embodiment 78, wherein the PD-1 binding antagonist is an anti-PD-1 antibody.

80. The method of embodiment 79, wherein the anti-PD-1 antibody is nivolumab, pembrolizumab, MEDI-0680, spartalizumab, cemiplimab, camrelizumab, sintilimab, tislelizumab, toripalimab, or dostarlimab.

81. The method of any one of embodiments 1-80, further comprising administering an additional therapeutic agent to the patient.

82. The method of embodiment 81, wherein the additional therapeutic agent is selected from the group consisting of an immunotherapy agent, a cytotoxic agent, a growth inhibitory agent, a radiation therapy agent, an anti-angiogenic agent, and combinations thereof.

83. A PD-1 axis binding antagonist for use in treatment of urothelial carcinoma in a patient in need thereof, wherein the treatment comprises administration of an effective amount of a treatment regimen comprising a PD-1 axis binding antagonist, wherein the treatment regimen is an adjuvant therapy, and wherein the patient has been identified as likely to benefit from the treatment regimen based on the presence of ctDNA in a biological sample obtained from the patient.

84. A PD-1 axis binding antagonist for use in treatment of urothelial carcinoma in a patient in need thereof, the treatment comprising:
(a) determining whether ctDNA is present in a biological sample obtained from the patient, wherein the presence of ctDNA in the biological sample indicates that the patient is likely to benefit from a treatment regimen comprising a PD-1 axis binding antagonist; and
(b) administering an effective amount of a treatment regimen comprising a PD-1 axis binding antagonist to the patient based on the presence of ctDNA in the biological sample, wherein the treatment regimen is an adjuvant therapy.

85. A PD-1 axis binding antagonist for use in treatment of a patient having a urothelial carcinoma who has been administered at least a first dose of a treatment regimen comprising a PD-1 axis binding antagonist, wherein the treatment regimen is a neoadjuvant therapy or an adjuvant therapy, and wherein ctDNA was present in a biological sample obtained from the patient prior to or concurrently with the first dose of the treatment regimen.

86. A pharmaceutical composition comprising a PD-1 axis binding antagonist for use in treatment of urothelial carcinoma in a patient in need thereof, wherein the treatment comprises administration of an effective amount of a treatment regimen comprising a PD-1 axis binding antagonist, wherein the treatment regimen is an adjuvant therapy, and wherein the patient has been identified as likely to benefit from the treatment regimen based on the presence of ctDNA in a biological sample obtained from the patient.

87. A pharmaceutical composition comprising a PD-1 axis binding antagonist for use in treatment of urothelial carcinoma in a patient in need thereof, the treatment comprising:
(a) determining whether ctDNA is present in a biological sample obtained from the patient, wherein the presence of ctDNA in the biological sample indicates that the patient is likely to benefit from a treatment regimen comprising a PD-1 axis binding antagonist; and
(b) administering an effective amount of a treatment regimen comprising a PD-1 axis binding antagonist to the patient based on the presence of ctDNA in the biological sample, wherein the treatment regimen is an adjuvant therapy.

88. A pharmaceutical composition comprising a PD-1 axis binding antagonist for use in treatment of a patient having a urothelial carcinoma who has been administered at least a first dose of a treatment regimen comprising a PD-1 axis binding antagonist, wherein the treatment regimen is a neoadjuvant therapy or an adjuvant therapy, and wherein ctDNA was present in a biological sample obtained from the patient prior to or concurrently with the first dose of the treatment regimen.

89. Use of a PD-1 axis binding antagonist in the manufacture of a medicament for treatment of urothelial carcinoma in a patient in need thereof, wherein the treatment comprises administration of an effective amount of a treatment regimen comprising a PD-1 axis binding antagonist, wherein the treatment regimen is an adjuvant therapy, and wherein the patient has been identified as likely to benefit from the treatment regimen based on the presence of ctDNA in a biological sample obtained from the patient.

90. Use of a PD-1 axis binding antagonist in the manufacture of a medicament for treatment of urothelial carcinoma in a patient in need thereof, the treatment comprising:
(a) determining whether ctDNA is present in a biological sample obtained from the patient, wherein the presence of ctDNA in the biological sample indicates that the patient is likely to benefit from a treatment regimen comprising a PD-1 axis binding antagonist; and
(b) administering an effective amount of a treatment regimen comprising a PD-1 axis binding antagonist to the patient based on the presence of ctDNA in the biological sample, wherein the treatment regimen is an adjuvant therapy.

91. Use of a PD-1 axis binding antagonist in the manufacture of a medicament for treatment of a patient having a urothelial carcinoma who has been administered at least a first dose of a treatment regimen comprising a PD-1 axis binding antagonist, wherein the treatment regimen is a neoadjuvant therapy or an adjuvant therapy, and wherein ctDNA was present in a biological sample obtained from the patient prior to or concurrently with the first dose of the treatment regimen.

92. An article of manufacture comprising a PD-1 axis binding antagonist and instructions to administer the PD-1 axis binding antagonist for treatment of urothelial carcinoma in a patient in need thereof, wherein the treatment comprises administration of an effective amount of a treatment regimen comprising a PD-1 axis binding antagonist, wherein the treatment regimen is an adjuvant therapy, and wherein the patient has been identified as likely to benefit from the treatment regimen based on the presence of ctDNA in a biological sample obtained from the patient.

93. An article of manufacture comprising a PD-1 axis binding antagonist and instructions to administer the PD-1 axis binding antagonist for treatment of urothelial carcinoma in a patient in need thereof, the treatment comprising:
(a) determining whether ctDNA is present in a biological sample obtained from the patient, wherein the presence of ctDNA in the biological sample indicates that the patient is likely to benefit from a treatment regimen comprising a PD-1 axis binding antagonist; and
(b) administering an effective amount of a treatment regimen comprising a PD-1 axis binding antagonist to the patient based on the presence of ctDNA in the biological sample, wherein the treatment regimen is an adjuvant therapy.

94. An article of manufacture comprising a PD-1 axis binding antagonist and instructions to administer the PD-1 axis binding antagonist for treatment of a patient having a urothelial carcinoma who has been administered at least a first dose of a treatment regimen comprising a PD-1 axis binding antagonist, wherein the treatment regimen is a neoadjuvant therapy or an adjuvant therapy, and wherein ctDNA was present in a biological sample obtained from the patient prior to or concurrently with the first dose of the treatment regimen.

Although the foregoing invention has been described in some detail by way of illustration and example for purposes of clarity of understanding, the descriptions and examples should not be construed as limiting the scope of the invention.

1. A method of treating muscle-invasive urothelial carcinoma (MIUC) in a patient in need thereof, the method comprising administering to the patient an effective amount of a treatment regimen comprising an anti-PD-L1 antibody, wherein the anti-PD-L1 antibody comprises (a) a hypervariable region (HVR)-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively, wherein the treatment regimen is an adjuvant therapy, and wherein the patient has been identified as likely to benefit from the treatment regimen based on the presence of circulating tumor DNA (ctDNA) in a biological sample obtained from the patient.

2. A method of treating MIUC in a patient in need thereof, the method comprising:
(a) determining whether ctDNA is present in a biological sample obtained from the patient, wherein the presence of ctDNA in the biological sample indicates that the patient is likely to benefit from a treatment regimen comprising an anti-PD-L1 antibody; and
(b) administering an effective amount of a treatment regimen comprising an anti-PD-L1 antibody to the patient based on the presence of ctDNA in the biological sample, wherein the treatment regimen is an adjuvant therapy, and wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

3-12. (canceled)

13. A method of monitoring the response of a patient having an MIUC who has been administered at least a first dose of a treatment regimen comprising an anti-PD-L1 antibody, wherein the treatment regimen is a neoadjuvant therapy or an adjuvant therapy, and wherein ctDNA was present in a pre-treatment biological sample obtained from the patient prior to or concurrently with the first dose of the treatment regimen, the method comprising determining whether ctDNA is present in a post-treatment biological sample obtained from the patient at a time point following administration of the first dose of the treatment regimen, thereby monitoring the response of the patient, wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

14-80. (canceled)

81. An article of manufacture comprising an anti-PD-L1 antibody and instructions to administer the anti-PD-L1 antibody for treatment of MIUC in a patient in need thereof, wherein the treatment comprises administration of an effective amount of a treatment regimen comprising an anti-PD-L1 antibody, wherein the anti-PD-L1 antibody comprises (a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and (b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively, wherein the treatment regimen is an adjuvant therapy, and wherein the patient has been identified as likely to benefit from the treatment regimen based on the presence of ctDNA in a biological sample obtained from the patient.

82-83. (canceled)

84. The method of claim 1, wherein:
(a) the biological sample is obtained prior to or concurrently with administration of a first dose of the treatment regimen;
(b) the biological sample is obtained within about 30 weeks from surgical resection;
(c) the biological sample is a blood sample, a plasma sample, a serum sample, a urine sample, a cerebrospinal fluid (CSF) sample, a nasal swab sample, a saliva sample, a stool sample, or a vaginal fluid sample;
(d) the benefit is in terms of improved disease-free survival (DFS), improved overall survival (OS), improved disease-specific survival, or improved distant metastasis-free survival;
(e) the presence of ctDNA is determined by a polymerase chain reaction (PCR)-based approach, a hybridization capture-based approach, a methylation-based approach, or a fragmentomics approach;
(f) the MIUC is muscle-invasive bladder cancer (MIBC) or muscle-invasive urinary tract urothelial cancer (muscle-invasive UTUC);
(g) the patient has previously been treated with neoadjuvant chemotherapy or has not received prior neoadjuvant chemotherapy;
(h) the patient has undergone surgical resection with lymph node dissection;
(i) the patient has no evidence of residual disease or metastasis as assessed by postoperative radiologic imaging;
(j) a tumor sample obtained from the patient has been determined to have a detectable expression level of PD-L1 in tumor-infiltrating immune cells that comprise about 1% or more of the tumor sample or less than 1% of the tumor sample;
(k) a tumor sample obtained from the patient has been determined to have a tissue tumor mutational burden (tTMB) score that is at or above a reference tTMB score;
(l) the patient has an increased expression level of one or more genes selected from PD-L1, IFNG, and CXCL9 relative to a reference expression level of the one or more genes in a further biological sample obtained from the patient;
(m) the patient has a decreased expression level of one or more pan-F-TBRS genes selected from ACTA2, ACTG2, TAGLN, TNS1, CNN1, TPM1, CTGF, PXDC1, ADAM12, FSTL3, TGFBI, and ADAM19 relative to a reference expression level of the one or more pan-F-TBRS genes in a further biological sample obtained from the patient;
(n) the patient's tumor has a basal-squamous subtype;
(o) the anti-PD-L1 antibody is atezolizumab; and/or
(p) the method further comprises administering an additional therapeutic agent to the patient.

85. The method of claim 84, wherein:
(a) the biological sample is obtained on cycle 1, day 1 (C1D1) of the treatment regimen;
(b) the biological sample is obtained within about 20 weeks from surgical resection;
(c) the biological sample is a plasma sample;
(d) the benefit is in terms of improved DFS or improved OS;
(e) improvement is relative to observation or relative to adjuvant therapy with a placebo;
(f) the presence of ctDNA is determined by a personalized ctDNA multiplexed polymerase chain reaction (mPCR) approach;
(g) the MIUC is histologically confirmed and/or wherein the patient has an Eastern Cooperative Oncology Group (ECOG) Performance Status of less than or equal to 2;
(h) the patient's MIUC at surgical resection is:
(i) ypT2-4a or ypN+ and M0; or
(ii) pT3-4a or pN+ and M0;
(i) the patient is cisplatin-ineligible or has refused cisplatin-based adjuvant chemotherapy;
(j) the surgical resection is cystectomy or nephroureterectomy;
(k) the tumor sample has been determined to have a detectable expression level of PD-L1 in tumor-infiltrating immune cells that comprise between about 1% to about 5% or about 5% or more of the tumor sample;
(l) the reference tTMB score is a pre-assigned tTMB score;
(m) the tumor sample is from surgical resection;
(n) the patient has an increased expression level of two or more genes selected from PD-L1, IFNG, and CXCL9 relative to a reference expression level of the two or more genes in the further biological sample obtained from the patient;
(o) the expression level of PD-L1, IFNG, and/or CXCL9 is an mRNA expression level;
(p) the patient has a decreased expression level of two, three, four, five, six, seven, eight, nine, ten, eleven, or all twelve of the pan-F-TBRS genes relative to a reference expression level of the two, three, four, five, six, seven, eight, nine, ten, eleven, or all twelve pan-F-TBRS genes in the further biological sample obtained from the patient;
(q) the expression level of the one or more pan-F-TBRS genes is an mRNA expression level;
(r) the patient's tumor has a basal-squamous subtype, and the patient has an increased expression level of one or more genes selected from CD44, KRT6A, KRT5, KRT14, COL17A1, DSC3, GSDMC, TGM1, and PI3 relative to a reference expression level of the one or more genes;
(s) the atezolizumab is administered intravenously:
(i) every two weeks at a dose of 840 mg;
(ii) every three weeks at a dose of 1200 mg; or
(iii) every four weeks at a dose of 1680 mg; and/or
(t) the additional therapeutic agent is an immunotherapy agent, a cytotoxic agent, a growth inhibitory agent, a radiation therapy agent, an anti-angiogenic agent, or combinations thereof.

86. The method of claim 85, wherein:
(a) the biological sample is obtained about 2 to about 20 weeks after surgical resection;
(b) the tumor sample has been determined to have a detectable expression level of PD-L1 in tumor-infiltrating immune cells that comprise between about 5% to about 10%, or about 10% or more of the tumor sample;
(c) the pre-assigned tTMB score is between about 8 and about 30 mutations per megabase (mut/Mb);
(d) the patient has an increased expression level of PD-L1, IFNG, and CXCL9 relative to a reference expression level of PD-L1, IFNG, and CXCL9 in the further biological sample obtained from the patient; and/or
(e) the atezolizumab is administered on Day 1 of each 28-day (±3 days) cycle for 12 cycles or one year, whichever occurs first.

87. The method of claim 86, wherein:
(a) the pre-assigned tTMB score is about 10 mut/Mb; and/or
(b) the further biological sample obtained from the patient is a tumor sample.

88. The method of claim 85, wherein:
(A) the personalized ctDNA mPCR approach comprises:
(a)
(i) sequencing DNA obtained from a tumor sample obtained from the patient to produce tumor sequence reads; and
(ii) sequencing DNA obtained from a normal tissue sample obtained from the patient to produce normal sequence reads;
(b) identifying one or more patient-specific variants by calling somatic variants identified from the tumor sequence reads and excluding germline variants or clonal hematopoiesis of indeterminate potential (CHIP) variants, wherein the germline variants or CHIP variants are identified from the normal sequence reads or from a publicly available database;
(c) designing an mPCR assay for the patient that detects a set of patient-specific variants; and
(d) analyzing the biological sample obtained from the patient using the mPCR assay to determine whether ctDNA is present in the biological sample;
(B) the personalized ctDNA mPCR approach is a SIGNATERA® ctDNA test or an ArcherDx Personalized Cancer Monitoring (PCM™) test; and/or
(C) the presence of at least one patient-specific variant in the biological sample identifies the presence of ctDNA in the biological sample.

89. The method of claim 88, wherein:
(a) the sequencing is whole-exome sequencing (WES) or whole-genome sequencing (WGS);
(b) the patient-specific variants are single nucleotide variants (SNVs) or short indels;
(c) the set of patient-specific variants comprises at least 2 patient-specific variants;
(d) analyzing the biological sample obtained from the patient using the mPCR assay comprises sequencing amplicons produced by the mPCR assay to identify patient-specific variants in the biological sample; and/or
(e) the presence of two patient-specific variants in the biological sample identifies the presence of ctDNA in the biological sample.

90. The method of claim 89, wherein:
(a) the sequencing is WES; and/or
(b) the set of patient-specific variants comprises 2 to 200 patient-specific variants.

91. The method of claim 90, wherein the set of patient-specific variants comprises 16 patient-specific variants.

92. The method of claim 13, wherein:
(a) an absence of ctDNA in the post-treatment biological sample obtained from the patient at a time point following administration of the first dose of the treatment regimen indicates that the patient is responding to the treatment regimen;
(b) the treatment regimen is an adjuvant therapy;
(c) the time point following administration of the first dose of the treatment regimen is on cycle 3, day 1 (C3D1) or cycle 5, day 1 (C5D1) of the treatment regimen;
(d) the pre-treatment biological sample obtained from the patient prior to or concurrently with a first dose of the treatment regimen and/or the post-treatment biological sample obtained from the patient at a time point following administration of the first dose of the treatment regimen is a blood sample, a plasma sample, a serum sample, a urine sample, a CSF sample, a nasal swab sample, a saliva sample, a stool sample, or a vaginal fluid sample;
(e) the presence of ctDNA is determined by a PCR-based approach, a hybridization capture-based approach, a methylation-based approach, or a fragmentomics approach;
(f) the MIUC is MIBC or muscle-invasive UTUC;
(g) the patient has not received prior neoadjuvant chemotherapy;
(h) the patient has no evidence of residual disease or metastasis as assessed by postoperative radiologic imaging;
(i) a tumor sample obtained from the patient has been determined to have a detectable expression level of PD-L1 in tumor-infiltrating immune cells that comprise about 1% or more of the tumor sample or less than 1% of the tumor sample;
(j) a tumor sample obtained from the patient has been determined to have a tTMB score that is at or above a reference tTMB score;
(k) the patient has an increased expression level of one or more genes selected from PD-L1, IFNG, and CXCL9 relative to a reference expression level of the one or more genes in a further biological sample obtained from the patient;
(l) the patient has a decreased expression level of one or more pan-F-TBRS genes selected from ACTA2, ACTG2, TAGLN, TNS1, CNN1, TPM1, CTGF, PXDC1, ADAM12, FSTL3, TGFBI, and ADAM19 relative to a reference expression level of the one or more pan-F-TBRS genes in a further biological sample obtained from the patient;
(m) the patient's tumor has a basal-squamous subtype;
(n) the anti-PD-L1 antibody is atezolizumab; and/or
(o) the method further comprises administering an additional therapeutic agent to the patient.

93. The method of claim 92, wherein:
(a) the pre-treatment biological sample obtained from the patient prior to or concurrently with a first dose of the treatment regimen and/or the post-treatment biological sample obtained from the patient at a time point following administration of the first dose of the treatment regimen is a plasma sample;
(b) the presence of ctDNA is determined by a personalized ctDNA mPCR approach;
(c) the MIUC is histologically confirmed and/or wherein the patient has an ECOG Performance Status of less than or equal to 2;
(d) the patient is cisplatin-ineligible or has refused cisplatin-based adjuvant chemotherapy;
(e) the patient's MIUC is pT3-4a or pN+ and M0 at surgical resection;
(f) the tumor sample has been determined to have a detectable expression level of PD-L1 in tumor-infiltrating immune cells that comprise between about 1% to about 5%, about 5% or more, or about 10% or more of the tumor sample;
(g) the reference tTMB score is a pre-assigned tTMB score;
(h) the tumor sample is from surgical resection;
(i) the patient has an increased expression level of two or more genes selected from PD-L1, IFNG, and CXCL9 relative to a reference expression level of the two or more genes in the further biological sample obtained from the patient;
(j) the expression level of PD-L1, IFNG, and/or CXCL9 is an mRNA expression level;
(k) the patient has a decreased expression level of two, three, four, five, six, seven, eight, nine, ten, eleven, or all twelve of the pan-F-TBRS genes relative to a reference expression level of the two, three, four, five, six, seven, eight, nine, ten, eleven, or all twelve pan-F-TBRS genes in the further biological sample obtained from the patient;
(l) the expression level of the one or more pan-F-TBRS genes is an mRNA expression level;
(m) the patient's tumor has a basal-squamous subtype, and the patient has an increased expression level of one or more genes selected from CD44, KRT6A, KRT5, KRT14, COL17A1, DSC3, GSDMC, TGM1, and PI3 relative to a reference expression level of the one or more genes;
(n) the atezolizumab is administered intravenously:
(i) every two weeks at a dose of 840 mg;
(ii) every three weeks at a dose of 1200 mg; or
(iii) every four weeks at a dose of 1680 mg; and/or
(o) the additional therapeutic agent is an immunotherapy agent, a cytotoxic agent, a growth inhibitory agent, a radiation therapy agent, an anti-angiogenic agent, or combinations thereof.

94. The method of claim 93, wherein:
(a) the tumor sample has been determined to have a detectable expression level of PD-L1 in tumor-infiltrating immune cells that comprise between about 5% to about 10%, or about 10% or more of the tumor sample;
(b) the pre-assigned tTMB score is between about 8 and about 30 mut/Mb;
(c) the patient has an increased expression level of PD-L1, IFNG, and CXCL9 relative to a reference expression level of PD-L1, IFNG, and CXCL9 in the further biological sample obtained from the patient; and/or
(d) the atezolizumab is administered on Day 1 of each 28-day (±3 days) cycle for 12 cycles or one year, whichever occurs first.

95. The method of claim 94, wherein:
(a) the pre-assigned tTMB score is about 10 mut/Mb; and/or
(b) the further biological sample obtained from the patient is a tumor sample.

96. The method of claim 93, wherein:
(A) the personalized ctDNA mPCR approach comprises:
(a)
(i) sequencing DNA obtained from a tumor sample obtained from the patient to produce tumor sequence reads; and
(ii) sequencing DNA obtained from a normal tissue sample obtained from the patient to produce normal sequence reads;
(b) identifying one or more patient-specific variants by calling somatic variants identified from the tumor sequence reads and excluding germline variants or CHIP variants, wherein the germline variants or CHIP variants are identified from the normal sequence reads or from a publicly available database;
(c) designing an mPCR assay for the patient that detects a set of patient-specific variants; and
(d) analyzing the pre-treatment and/or the post-treatment biological samples obtained from the patient using the mPCR assay to determine whether ctDNA is present in the pre-treatment and/or the post-treatment biological samples;
(B) the personalized ctDNA mPCR approach is a SIGNATERA® ctDNA test or an ArcherDx PCM™ test; and/or
(C) the presence of at least one patient-specific variant in the pre-treatment and/or the post-treatment biological samples identifies the presence of ctDNA in the pre-treatment and/or the post-treatment biological samples.

97. The method of claim 96, wherein:
(a) the sequencing is WES or WGS;
(b) the patient-specific variants are SNVs or short indels;
(c) the set of patient-specific variants comprises at least 2 patient-specific variants;
(d) analyzing the pre-treatment and/or the post-treatment biological samples obtained from the patient using the mPCR assay comprises sequencing amplicons produced by the mPCR assay to identify patient-specific variants in the pre-treatment and/or the post-treatment biological samples; and/or
(e) the presence of two patient-specific variants in the pre-treatment and/or the post-treatment biological samples identifies the presence of ctDNA in the pre-treatment and/or the post-treatment biological samples.

98. The method of claim 97, wherein:
(a) the sequencing is WES; and/or
(b) the set of patient-specific variants comprises 2 to 200 patient-specific variants.

99. The method of claim 98, wherein the set of patient-specific variants comprises 16 patient-specific variants.


SEQUENCE LISTING (10 sequences)

SEQ ID NO: 1 - protein, 447 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, synthetic construct
GFTFSDSWIH

SEQ ID NO: 4 - protein, 18 aa, synthetic construct
AWISPYGGSTYYADSVKG

SEQ ID NO: 5 - protein, 9 aa, synthetic construct
RHWPGGFDY

SEQ ID NO: 6 - protein, 11 aa, synthetic construct
RASQDVSTAVA

SEQ ID NO: 7 - protein, 7 aa, synthetic construct
SASFLYS

SEQ ID NO: 8 - protein, 9 aa, synthetic construct
QQYLYHPAT

SEQ ID NO: 9 - protein, 118 aa, synthetic construct [sequence omitted]

SEQ ID NO: 10 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR